SEQUENCE LISTING (196 sequences)

SEQ ID NO: 1 - DNA, 1169 bp, Arabidopsis thaliana
  misc_feature (1)..(1169): Ceres SEED LINE. ME06634
tcgtttgtgtttttgattggcggagaattggtgatagataagcttcttcttccctcttctcaacttggtggatctgtcatcgatggcctctttgatgctcaacggggccattaccttccccaaaggtttaggttcccctggttccaatttgcatgccagatcgattcctcggccgaccttactctcagttacccgaacctccacacctagactctcggtggctactagatgcagcagcagcagcgtgtcgtcttcccggccatcggcgcaacctaggttcattcagcacaagaaggaggcttactggttctacaggttcttatccatcgtatacgaccatgtcatcaatcctgggcattggaccgaggatatgagagacgacgctcttgagccagcggatctcagccatccggacatgcgagtggtcgatgtcggcggcggaactggtttcactactctgggcatagtcaagacagtgaaggccaagaatgtgaccattctggaccagtcgccacatcagctggccaaagcaaagcaaaaggagccgttgaaagaatgcaagatcgtcgagggagatgctgaggatcttccttttccaaccgattatgctgacagatacgtttctgctggaagcattgagtactggccggacccgcagaggggaataagggaagcgtacagggttctcaagatcggtggcaaagcgtgtctcatcggccctgtctacccaaccttctggctctctcgcttcttttctgatgtctggatgctcttccccaaggaggaagaatacattgagtggttcaagaatgccggtttcaaggacgttcagctcaagaggattggccccaagtggtaccgtggtgttcgcaggcacggccttatcatgggatattctgtcactggtgttaaacctgcctccggtgactctcctctccagcttggtccaaaggaagaggacgtagagaagcctgtcaacaaccccttctccttcttgggacgcttcctcctgggaactctagcagctgcctggtttgtgttaatccctatctacatgtggatcaaggatcagatcgttcccaaagaccaacccatctgatccttctcttctaggacatgatcattgtatcattgtaaacccctcttgtggtaaagaaagattcgagtcc

SEQ ID NO: 2 - protein, 338 aa, Arabidopsis thaliana
  misc_feature (1)..(338): Ceres SEED LINE. ME06634
MASLMLNGAITFPKGLGSPGSNLHARSIPRPTLLSVTRTSTPRLSVATRCSSSSVSSSRPSAQPRFIQHKKEAYWFYRFLSIVYDHVINPGHWTEDMRDDALEPADLSHPDMRVVDVGGGTGFTTLGIVKTVKAKNVTILDQSPHQLAKAKQKEPLKECKIVEGDAEDLPFPTDYADRYVSAGSIEYWPDPQRGIREAYRVLKIGGKACLIGPVYPTFWLSRFFSDVWMLFPKEEEYIEWFKNAGFKDVQLKRIGPKWYRGVRRHGLIMGYSVTGVKPASGDSPLQLGPKEEDVEKPVNNPFSFLGRFLLGTLAAAWFVLIPIYMWIKDQIVPKDQPI

SEQ ID NO: 3 - protein, 337 aa, Zea mays
  misc_feature (1)..(337): Ceres CLONE ID no. 1061027
MASLMLNGAITFPKGLGFPASNLHARPSPPLSLVSNTATRRLSVATRCSSSSSVSASRPSAQPRFIQHKKEAYWFYRFLSIVYDHIINPGHWTEDMRDDALEPADLSHPDMRVVDVGGGTGFTTLGIVKTVKAKNVTILDQSPHQLAKAKQKEPLKECKIVEGDAEDLPFPTDYADRYVSAGSIEYWPDPQRGIREAYRVLKIGGKACLIGPVHPTFWLSRFFADVWMLFPKEEEYIEWFKNAGFKDVQLKRIGPKWYRGVRRHGLIMGCSVTGVKPASGDSPLQLGPKEEDVEKPVNNPFSFLGRFLLGTLAAAWFVLIPIYMWIKDQIVPKDQPI

SEQ ID NO: 4 - protein, 342 aa, Glycine max
  misc_feature (1)..(342): Ceres CLONE ID no. 480158
MASVMLSGTEKLTLRTLTGNGLGFTGSDLHGKNFPRVSFVATTSAKVPNFRSLVVPKCSVSASRPTSQPRFIQHKKEAFWFYRFLSIVYDHVINPGHWTEDMRDDALEPADLNDRNMIVVDVGGGTGFTTLGIVKHVDAKNVTILDQSPHQLAKAKQKEPLKECKIIEGDAEDLPFRTDYADRYVSAGSIEYWPDPQRGIKEAYRVLKLGAKACLIGPVYPTFWLSRFFADVWMLFPKEEEYIEWFQKAGFKDVQLKRIGPKWYRGVRRHGLIMGCSVTGVKPASGDSPLQLGPKEEDVEKSVNPFVFALRFVLGALAATWFVLVPIYMWLKDQVVPKGQPI

SEQ ID NO: 5 - protein, 343 aa, Glycine max
  misc_feature (1)..(343): Ceres CLONE ID no. 656984
MASLMLNGAHQNPNLISGIAPNGLNFHNKCLFQKGVLSHGNKLRVVRKNFTPKGSMSASSSSSSRPGSQPRFIQHKKEAFWFYRFLSIVYDHIINPGHWTEDMRDEALEPADLYNRNLRVVDVGGGTGFTTLGIVKHVDAKNVTILDQSPHQLAKAKQKEPLKECKIVEGDAEDLPFPTDYADRYVSAGSIEYWPDPQRGITEAYRVLRIGGIACVIGPVHPTFWLSRFFADVWMLFPKEEEYIEWFKKAGFKDVKLKRIGPKWYRGVRRHGLIMGCSVTGVKPLSGDSPLQLGPKVEDVKKPVNPFVFLYRFILGTIASTYFVLVPIYMWIKDKIVPRGMPI

SEQ ID NO: 6 - protein, 330 aa, Oryza sativa
  misc_feature (1)..(330): Public GI no. 50934645
MKEMVSSSTFRAPGGLGFLGPSKIGLIPLRNRSGVRSRVKYIAPKCAVSSARPASQPRFIQHKKEAFWFYRFLSIVYDHVINPGHWTEDMRDDALEPAELYHHGLKVVDVGGGTGFTTLGIVKHVDNENVTLLDQSPHQLEKARQKVALNGVNIIEGDAEDLPYPTDTFDRYVSAGSIEYWPDPQRGIREAYRVLKLGGVACLIGPVHPTFWLSRFFADMWMLFPKEEEYIEWFQKAGFQDVKIKRIGPKWYRGVRRHGLIMGCSVTGVKRSSGDSPLQLGPKAEDVEKPVNPFTFIFRFVMGTICASYYVLVPIYMWMKDQIVPKDQPI

SEQ ID NO: 7 - protein, 335 aa, Nicotiana tabacum
  misc_feature (1)..(335): Public GI no. 1419090
MASSILSGAENFKILSGISPSELHIKCFPQKGLVNYSRIPNTKSRTLRTKCSVSSSRPASQPRFIQHKKEAFWFYRFLSIVYDHVINPGHWTEDMRDEALEPAELNSRQLQVVDVGGGTGFTTLGIVKHVDAKNVTIIDQSPHQLAKAREKEPLKECKILEGDAEDLPFPTDTFDRYVSAGSIEYWPDPQRGIKEAYRVLTIGGVACLIGPVYPTFWLSRFFADMWMLFPKEEEYIEWFKKAGFAQVKLKRIGPKWYRGVRRHGLIMGCSVTGVKPYFGESPLQLGPKVEDVSKPVNPFAFLVRFLLGITAATYYVLVPIYMWLKDQITPKGQPI

SEQ ID NO: 8 - protein, 344 aa, Spinacia oleracea
  misc_feature (1)..(344): Public GI no. 21228
MACSMLNGVDKLALISGKTPNRLRFSGSDFTGSYKLPRLNLPPNSRNLRAKTLTTVTKCTLSASERPASQPRFIQNKQEAFWFYRFLSIVYDNIINPGHWTEDMRDVALEPADLNNRNMLVVDVGGGTGFTTLGIIKHVDPKNVTILDQSPHQLAKAKAKKPLKECRIIEGDAEDLPFPTDYADRYVSAGSIEYWPDPQRGIREAYRVLKLGGKACLIGPVYPTFWLSRFFADVWMLFPKEEEYIEWFQKAGFKDVQLKRIGPKWYRGVRRHGLIMGCSVTGVKPASGDSPLQLGPKVEDVQKPVHPLVFLYRFLLGALASTYYVLVPIYMWIKDKIFPKGMPL

SEQ ID NO: 9 - protein, 337 aa, Chlamydomonas reinhardtii
  misc_feature (1)..(337): Public GI no. 37265798
MHAANPSQRAALSQRADAANAHQRLALPLSSRPLHRSSVFSSRRLPILQAGSTDTYSRPISAPRLVQHKSEAYWFYAGLSQVYDHIVNPGHWTEDMRDDALAPAKLDDPNLKVVDVGGGTGFCTLGVVKTVKPENVTLMDQSPHQLAKAKAKPALKGVTILEGDAEDLPFPTDTFDRYVSAGSIEYWPEPQRGIREAYRVVKEGGLACMIGPVHPTHPVSRFFADAWMLFPTEEEYIEWFTKAGFTDVKMTRIGPKWYRGVRRHGLIMGCSVTGVKPKAGDSPLVMGPKAEVSGKMNTNPLSFLLNLILGTAAGFYYFCLPIYMYIKNLIWPKNWEM

SEQ ID NO: 10 - protein, 352 aa, Zea mays
  misc_feature (1)..(352): SEQ ID NO 22 of patent application 20030150015
MAMASTYAPGGGARALAQGRCRVRGPAGLGFLGPSKAAGLPRPLALALARRMSSPVAVGARLRCAASSSPAAARPATAPRFIQHKKEAFWFYRFLSIVYDHVINPGHWTEDMRDDALEPADLFSRHLTVVDVGGGTGFTTLGIVKHVNPENVTLLDQSPHQLDKARQKEALKGVTIMEGDAEDLPFPTDSFDRYISAGSIEYWPDPQRGIKEAYRVLRFGGLACVIGPVYPTFWLSRFFADMWMLFPKEEEYIEWFKKAGFRDVKLKRIGPKWYRGVRRHGLIMGCSVTGVKRERGDSPLELGPKAEDVSKPVNPITFLFRFLVGTICAAYYVLVPIYMWIKDQIVPKGMPI

SEQ ID NO: 11 - protein, 341 aa, Gossypium hirsutum
  misc_feature (1)..(341): SEQ ID NO 23 of patent application 20030150015
MASSMLNGAETFTLIRGVTPKSIGFLGSGLHGKQFSSAGLIYSPKMSRVGTTIAPRCSLSASRPASQPRFIQHKKEAFWFYRFLSIVYDHVINPGHWTEDMRDDALEPADLNDRDMVVVDVGGGTGFTTLGIVQHVDAKNVTILDQSPHQLAKAKQKEPLKECNIIEGDAEDLPFPTDYADRYVSAGSIEYWPDPQRGIKEAYRVLKQGGKACLIGPVYPTFWLSRFFADVWMLFPKEEEYIEWFEKAGFKDVQLKRIGPKWYRGVRRHGLIMGCSVTGVKPASGDSPLQLGPKAEDVSKPVNPFVFLLRFMLGATAAAYYVLVPIYMWLKDQIVPEGQPI

SEQ ID NO: 12 - protein, 344 aa, Allium porrum
  misc_feature (1)..(344): SEQ ID NO 24 of patent application 20030150015
MASSMLSGAESLSMLRIHHQPKLTFSSPSLHSKPTNLKMDLIPFATKHQKTKKASIFTCSASSSSRPASQPRFIQHKQEAFWFYRFLSIVYDHVINPGHWTEDMRDDALEPAELYDSRMKVVDVGGGTGFTTLGIIKHIDPKNVTILDQSPHQLEKARQKEALKECTIVEGDAEDLPFPTDTFDRYVSAGSIEYWPDPQRGIKEAYRVLKLGGVACLIGPVHPTFWLSRFFADMWMLFPTEEEYIEWFKKAGFKDVKLKRIGPKWYRGVRRHGLIMGCSVTGVKRLSGDSPLQLGPKAEDVKKPINPFSFLLRFILGTIAATYYVLVPIYMWIKDQIVPKGQPI

SEQ ID NO: 13 - protein, 342 aa, Glycine max
  misc_feature (1)..(342): SEQ ID NO 25 of patent application 20030150015
MGSVMLSGTEKLTLRTLTGNGLGFTGSDLHGKNFPRVSFAATTSAKVPNFRSIVVPKCSVSASRPSSQPRFIQHKKEAFWFYRFLSIVYDHVINPGHWTEDMRDDALEPADLNDRNMIVVDVGGGTGFTTLGIVKHVDAKNVTILDQSPHQLAKAKQKEPLKECKIIEGDAEDLPFRTDYADRYVSAGSIEYWPDPQRGIKEAYRVLKLGGKACLIGPVYPTFWLSRFFADVWMLFPKEEEYIEWFQKAGFKDVQLKRIGPKWYRGVRRHGLIMGCSVTGVKPASGDSPLQLGPKEEDVEKPVNPFVFALRFVLGALAATWFVLVPIYMWLKDQVVPKGQPI

SEQ ID NO: 14 - protein, 348 aa, Oryza sativa
  misc_feature (1)..(348): SEQ ID NO 26 of patent application 20030150015
MAMASSAYAPAGGVGTHSAPGRIRPPRGLGFSTTTTKSRPLVLTRRGGGGGNISVARLRCAASSSSAAARPMSQPRFIQHKKEAFWFYRFLSIVYDHVINPGHWTEDMRDDALEPADLYSRKLRVVDVGGGTGFTTLGIVKRVDPENVTLLDQSPHQLEKAREKEALKGVTIMEGDAEDLPFPTDTFDRYVSAGSIEYWPDPQRGIKEAYRVLRLGGVACMIGPVHPTFWLSRFFADMWMLFPKEEEYIEWFKKAGFKDVKLKRIGPKWYRGVRRHGLIMGCSVTGVKREHGDSPLQLGPKVEDVSKPVNPITFLFRFLMGTICAAYYVLVPIYMWIKDQIVPKGMPI

SEQ ID NO: 15 - protein, 337 aa, Brassica napus; identical to SEQ ID NO: 3
  misc_feature (1)..(337): SEQ ID NO 27 of patent application 20030150015

SEQ ID NO: 16 - DNA, 1562 bp, Gossypium hirsutum
  misc_feature (1)..(1562): Ceres CLONE ID no. 183492
agagagcagagtccagtccaaaagctgtggtgtccgtttcctttaagtctcaaccaaagttgcagaaagccagacgctaaaatactaaaactagctagcccaaaacggtaccctttttttgctccaatctcgtttacccttaaggtgtttatcttggtaattgatattttgctcaaatctttcaacaagtaaagattgcagcttttcaaatcgtgggttttttttttttttgcttgcttaatccatggcttcttccatgctcaatggagcagaaaacttcactctcattaggggggtaaccccaaataggattgattttttagggtcaggttttcatgggaaacacttgtccaatttgggtttagcctctagtgcgaggatctccaggccaggaacaacaatggctccaaagtgcggcttatcagcatcaaggccagcttcacaaccaagattcatacaacacaaaaaggaggctttttggttctacaggttcctctcaattatatatgaccatgtgataaaccctggtcactggactgaagacatgagggatgatgcacttgagccagccgatcttaacaacaggaacatgattgttgtagatgttggtggtggaactggtttcactactttgggtatcgttaagcatgtggatgctaagaacgttacaattctcgaccaatctccgcaccagcttgccaaggctaagcaaaaggagtctctgaaggagtgcaggataattgaaggtgatgcagaagatctccccttccgtactgattatgccgacagatatgtgtctgctggcagcatagagtactggccagacccacaacggggcatcaaggaagcatacagggtgttgaaactaggaggaaaagcttgtctgattggtcctgtgtaccctacattctggttgtcacgcttctttgcggatgtttggatgcttttccctaaggaggaagagtacattgagtggtttgaaaaggctggatttaaggatgtccaactcaaaaggattggccctaagtggtatcgtggagttcgcagacatggtttgatcatggggtgctctgtaactggtgtcaaacctgcatctggggactctcctttgcagcttggcccgaaggcagaggatgtatcgaaaccgataaacccattaacattcctgttgcgctttatactgggtaccatggcagctacgtattatgtattggtacctatctatatgtggttgaaggatcaaattgtgccggaaggtcaaccaatctaaacagcggccatacctgctgctgcccttccaaattgcctatatttctctagtcatagcgtatgtaatgcaagacatcttctagctatgtattttatcatctaatattatcctgtttgtgattcatatcttcaagcctgttttcatgtaacatattagctttctgttttctttgttcagagctgaattatcatagataaacaagagattagaaaataaaaaaagtccagagatcaccctggggatcaattcagcagatcagcctctatgaacaagtttaccatatcagctttcc

SEQ ID NO: 17 - protein, 341 aa, Gossypium hirsutum
  misc_feature (1)..(341): Ceres CLONE ID no. 183492
MASSMLNGAENFTLIRGVTPNRIDFLGSGFHGKHLSNLGLASSARISRPGTTMAPKCGLSASRPASQPRFIQHKKEAFWFYRFLSIIYDHVINPGHWTEDMRDDALEPADLNNRNMIVVDVGGGTGFTTLGIVKHVDAKNVTILDQSPHQLAKAKQKESLKECRIIEGDAEDLPFRTDYADRYVSAGSIEYWPDPQRGIKEAYRVLKLGGKACLIGPVYPTFWLSRFFADVWMLFPKEEEYIEWFEKAGFKDVQLKRIGPKWYRGVRRHGLIMGCSVTGVKPASGDSPLQLGPKAEDVSKPINPLTFLLRFILGTMAATYYVLVPIYMWLKDQIVPEGQPI

SEQ ID NO: 18 - DNA, 1222 bp, Gossypium hirsutum
  misc_feature (1)..(1222): Ceres CLONE ID no. 1925254
attttgctcaaatctttcagcaagtaaatattgcagcttttaaaattgtgggttttttttttcttgcttaatccatggcttcttccatgcttaatggagcaaaaagcttcactctcattaggggggtaaccccaagaagggttgatttttttgggtcaggttttcatgggaaacacttatctaatttgggtttagcctttagtgtgaggatctccaggccaggaacaacaatggctccaaagtgcggcttatcagcatcaaggccagcttcacaaccacgattcatacaacacaaaaaagaggctttttggttctacaggttcctctcaattatatatgaccatgtgataaaccctggtcactggactgaagacatgaggaatgatgcacttgagccagccgatctcaacaacaggaacatgattgttgtagatgttggtggtggaactggtttcactactttgggtatcgttaagcatgtggatgctaagaatgttacaattctcgaccaatctccgcaccagcttgccaaggctaagcaaaaggagcctttgaaggagtgcaggataattgaaggtgatgcagaagatctccacttccgtactgattatgccgacagatatgtgtctgctggcagcatagagtactggccagacccacaacggggcatcaaggaagcatatagggtattgaaactaggaggaaaagcttgtctgattggtcctgtgtacccgacattctggttgtcacgcttctttgcggatgtttggatgcttttccctaaggaggaagagtacattgactggtttgaaaaggctggatttaaggatgtccaactcaaaaggattggccctaagtggtatcgtggagttcgcagacatggtttgatcatggggtgctctgtaactggcgtcaaacctgcatctggggactctcctttgcagcttggcccgaaggcagaggatgtatcgaaaccaataaacccattaacattcctgttgcgctttatactgggtaccatggcagctacgtattatgtattggtccctatctatatgtggttgaaggatcaaattgtgccggaaggtcaaccaatctaaacagcggccgtacctgctgctgcccttccaagttgcctatatttctttagtcatagcgtatgtaatgcaagacatcttctagctatgtgttttatcatctaatattatcctgttcttaattc

SEQ ID NO: 19 - protein, 341 aa, Gossypium hirsutum
  misc_feature (1)..(341): Ceres CLONE ID no. 1925254
MASSMLNGAKSFTLIRGVTPRRVDFFGSGFHGKHLSNLGLAFSVRISRPGTTMAPKCGLSASRPASQPRFIQHKKEAFWFYRFLSIIYDHVINPGHWTEDMRNDALEPADLNNRNMIVVDVGGGTGFTTLGIVKHVDAKNVTILDQSPHQLAKAKQKEPLKECRIIEGDAEDLHFRTDYADRYVSAGSIEYWPDPQRGIKEAYRVLKLGGKACLIGPVYPTFWLSRFFADVWMLFPKEEEYIDWFEKAGFKDVQLKRIGPKWYRGVRRHGLIMGCSVTGVKPASGDSPLQLGPKAEDVSKPINPLTFLLRFILGTMAATYYVLVPIYMWLKDQIVPEGQPI

SEQ ID NO: 20 - DNA, 1296 bp, Panicum virgatum
  misc_feature (1)..(1296): Ceres CLONE ID no. 1792831
agtcatccaccactcgctcgcccgcgccattccccttccccctcctcgtcgcggcgccgcggcggcgaagggcgaaggcaatggcgatggcctccacctacgcaccgggcggcggcgcgggggcgctcgcgccgggaagggccagggtccgcgagcccgccggggggctcgtcctcggcccctccaaggccggcctcccccgccccctcgccctcgccaggcggagcccccagctcgcggccgccaggctgcggtgcgcggcgtcctcgtcgcccgcggcccccgcgcggcccgtcacggcgccgcggttcatccagcacaagaaggaggccttctggttctaccgcttcctctccatcgtctacgaccacgtcatcaacccgggccactggaccgaggacatgcgcgacgacgcgctcgagcccgccgacctctacagccgccacctcaaggtcgtcgacgtcggcggcggcaccggcttcaccacgctcgggatcgtcaagcacatcaacccggagaacgtcacgctgctcgaccagtccccgcaccagctcgagaaggccaggcagaaggaggcgctcaagggggtcaccatcatggagggcgacgccgaggacctgcccttcccgaccgacagcttcgaccgatacatctccgccggcagcatcgagtattggcctgatccgcagagaggaatcaaggaagcgtacagggtcctgaggtttggtgggacagcttatgtgatcggccctgtgcacccaaccttctggctctcccgcttcttcgccgacatgtggatgctcttccccaagaaagaagagtacatcgagtggttcaagaaggctgggttcaaggatgtcaagctgaaaaggattggaccaaagtggtaccgtggcgtccgaaggcatggcctgatcatgggatgctctgtcacaggtgtgaagagagaacgtggagactctcctttggagcttggtccgaaggctgaggatgtcagcaagccagtgaatcctatcacgttcctctttcgcttcctcataggaacaatatgtgctgcatactttgttctggtgcctatttacatgtggataaaggaccagatcgtgcccaaaggcatgccaatctgagtgaagggggctgagctcaaggaagagaggatagccaatatctcaattagtcttctctatattaggcgcttcctgtctgttttgtatttacctttcatttttcttctgttatgtatttcttctactgttgttcctgtttgtgcgaagcaataatattaagaccttgatc

SEQ ID NO: 21 - protein, 348 aa, Panicum virgatum
  misc_feature (1)..(348): Ceres CLONE ID no. 1792831
MAMASTYAPGGGAGALAPGRARVREPAGGLVLGPSKAGLPRPLALARRSPQLAAARLRCAASSSPAAPARPVTAPRFIQHKKEAFWFYRFLSIVYDHVINPGHWTEDMRDDALEPADLYSRHLKVVDVGGGTGFTTLGIVKHINPENVTLLDQSPHQLEKARQKEALKGVTIMEGDAEDLPFPTDSFDRYISAGSIEYWPDPQRGIKEAYRVLRFGGTAYVIGPVHPTFWLSRFFADMWMLFPKKEEYIEWFKKAGFKDVKLKRIGPKWYRGVRRHGLIMGCSVTGVKRERGDSPLELGPKAEDVSKPVNPITFLFRFLIGTICAAYFVLVPIYMWIKDQIVPKGMPI

SEQ ID NO: 22 - DNA, 1170 bp, Panicum virgatum
  misc_feature (1)..(1170): Ceres CLONE ID no. 1804277
atatactctatccgagatcgctcgcccttcccctccccacttctcgtctccgcgcggcggcagcgaggaaatcgcgatggcctccacctacgggccgggcggcggcgccaagctgaggtgcgcggcgtcctcgtcggcggcagccccggcgcggcccgtcaaggcgccgcggttcatccagcacaagaaggaggccttctggttctaccgcttcatctccgtcgcctacgacaacgtcttcaacccgggccacttcactgagaacatgcgcgacgacgcgctcgagcccgccgacctctacagcagccacctcaaggtcgtcgacgtcggcggcggcacgggcttcaccacgctcgggatcgtcaagcacgtcgacccggagaacgtcacgctgctcgaccagtccccgcaccagctcgagaaggccaggcagaaagtggcgctcaagggggtcaccatcatggagggcgacgccgaggacctgcccttccccaccgacaccttcgaccgatacgtctccgccggcagcattgagtattggcctgatccacagagaggaatcaaggaggcatacagggtcctgaagtttggtgggacagcttgtgtgattggcccggtgtacccaaccttctggctgtcccgcttcttcgccgacatgtggatgcttttcccccaggaagaagagtatatcgagtggttcaagaaggctgggtttaaggatgtcaagctgaaaagaattggaccaaagtggtaccgtggtgtccggaggcatggcctgatcattggatgctctgtcacaggtgtgaagagagaacgtggagactcttctttggagcttggtccgaaagctgaggatgtcagcaaaccagtgaatccaatcaccttcctctttcgcttcctcataggaacaatatgtgctgcatactatgttctggtgcctatttacatgtggataaaggacaagattgtgcccaaaggcatgccaatctgagtgaaggggacttagatgaaagcctgcaaggaagagagggtagccaatatctgaattaggcttctctatagtaggcgcttcctgtgtgttttgtattgtatttatctttcctcctgttatgtacttcttttactgttgttcctgtttgtgtgaatataattaatattattaatagtggc

SEQ ID NO: 23 - protein, 304 aa, Panicum virgatum
  misc_feature (1)..(304): Ceres CLONE ID no. 1804277
MASTYGPGGGAKLRCAASSSAAAPARPVKAPRFIQHKKEAFWFYRFISVAYDNVFNPGHFTENMRDDALEPADLYSSHLKVVDVGGGTGFTTLGIVKHVDPENVTLLDQSPHQLEKARQKVALKGVTIMEGDAEDLPFPTDTFDRYVSAGSIEYWPDPQRGIKEAYRVLKFGGTACVIGPVYPTFWLSRFFADMWMLFPQEEEYIEWFKKAGFKDVKLKRIGPKWYRGVRRHGLIIGCSVTGVKRERGDSSLELGPKAEDVSKPVNPITFLFRFLIGTICAAYYVLVPIYMWIKDKIVPKGMPI

SEQ ID NO: 24 - DNA, 994 bp, Arabidopsis thaliana
  misc_feature (1)..(994): Ceres SEED LINE. ME04024
attcccacttccacacatacacatatacaacagagcaagagagtcaatcaagtagagtgaagatggcaactaaacaagaagctttagccatcgatttcataagccaacaccttctcacagactttgtttccatggaaactgatcacccatctctttttaccaaccaacttcacaactttcactcagaaacaggccctagaaccatcaccaaccaatcccctaaaccgaattcgactcttaaccagcgtaaaccgcccttaccgaatctatccgtctcgagaacggtttcaacaaagacagagaaagaggaagaagagaggcactacaggggagtgagacgaagaccgtggggaaaatacgcggcggagattagggatccgaacaaaaagggttgtaggatctggcttgggacttacgacactgccgtggaagctggaagagcttatgaccaagcggcgtttcaattacgtggaagaaaagcaatcttgaatttccctctcgatgttagggttacgtcagaaacttgttctggggaaggagttatcggattagggaaacgaaagcgagataagagttctccgccggaagaggagaaggcggctagggttaaagtggaggaagaagagagtaatacgtcggagacgacggaggctgaggttgagccggtggtaccattgacgccgtcaagttggatggggttttgggatgtgggagcaggagatggtattttcagtattcctccgttatctccgacgtctcccaacttttccgttatctccgtcacttaaaacttcggaaaagtcaacgtacgatgacgttttcacttgcgtcactctcatgatttcatttattcttgtataatataaaggtagcggtagtgtgcaaatatcaaataagtagtttaattagtaccaatcattttattcattattttttttagtagaatatttggatgttgaaaatataaatttaattttgtatttgttgatgttac

SEQ ID NO: 25 - protein, 241 aa, Arabidopsis thaliana
  misc_feature (1)..(241): Ceres SEED LINE. ME04024
MATKQEALAIDFISQHLLTDFVSMETDHPSLFTNQLHNFHSETGPRTITNQSPKPNSTLNQRKPPLPNLSVSRTVSTKTEKEEEERHYRGVRRRPWGKYAAEIRDPNKKGCRIWLGTYDTAVEAGRAYDQAAFQLRGRKAILNFPLDVRVTSETCSGEGVIGLGKRKRDKSSPPEEEKAARVKVEEEESNTSETTEAEVEPVVPLTPSSWMGFWDVGAGDGIFSIPPLSPTSPNFSVISVT

SEQ ID NO: 26 - protein, 214 aa, Brassica napus
  misc_feature (165)..(165): Xaa is any aa, unknown, or other
MASSQDQSALDLITQHLLTDFPSLETFVSSIHQSTTSTLSQRKPSLATISVPTTAPVVQEDDHRHYRGVRRRPWGKYAAEIRDPNKKGVRVWLGTFDTAVEAARGYDRAAFKLRGSKAILNFPLEAGKHEDNNTVALKSKRKRPETQDENHGRNLISHKAVIREXTEAQGEACPLTPSSWMGFWDGVDGIGTGLXSXPPLXPYPSXGHXQLGVK

SEQ ID NO: 27 - protein, 244 aa, Lycopersicon esculentum
  misc_feature (1)..(244): Public GI no. 45642990
MGSPQETCTSLDLIRQHLFDESLDQTCFSFETTQTSNLDDIASFFNATSKTEYDGFFEFEAKRHVIHSNSPKQSNLRERKPSLNVAIPAKPVVVVENVEIEKKHYRGVRQRPWGKFAAEIRDPNRKGTRVWLGTFDTAVDAAKAYDRAAFKLRGSKAILNFPLEVANFKQQNDETKTETKSSGSKRVRGETEELVIKKERKIEEERVLPTAAAPLTPSSWSTIWDEKGIFEVPPLSPLSQLVMI

SEQ ID NO: 28 - protein, 278 aa, Vitis aestivalis
  misc_feature (1)..(278): Public GI no. 40060531
MGEEASSLQLIHHLLLSDFDSMETFVSHVSHSLRSSASDSSVSTDDIIQVSEYPKLHEDESNAFLFDYSTSSPSAVFQFQTESPKPSRLSHRRPPVSISLPPPPISHTSSSLDSGERRHYRGVRRRPWGKFAAEIRDPNRRGSRVWLGTFETAIEAARAYDRAAFKMRGSKAVLNFPLEAGNWSDSDPPATSIRKRERESESEEREQPEIKVLKQEEASPDSDSPVVAEAANVLEASPLTPSSWRTVWEERDMDGAFHMPPLTPLSPHPWIGYSRLIS

SEQ ID NO: 29 - protein, 218 aa, Sisymbrium irio
  misc_feature (1)..(218): Public GI no. 38260618
MAAFEESTDLDAIQGHLFEDFMVSDGFMGDFDFNASFVSGLWCIEPVMNQVPKQEPDSPVLDPDSFVKEFLQVEAESSTSTGTTELNSSSQETDQSISTRKKSKRFEEQEEEEPRHYRGVRRRPWGKFAAEIRDPAKKGSRIWLGTFESDVDAARAYDCAAFKLRGRKAVLNFPLDAGKYEAPANSGRKRKRSDVQGELQRSQSNSSSSSSDGETTCE

SEQ ID NO: 30 - protein, 266 aa, Glycine max
  misc_feature (1)..(266): Ceres CLONE ID no. 548557
MQSSISQSEICITDYLLPQEVPSQFQFPDMSNNNIPMNHTNLQMPQITSFSKPPRSSSNLSNRKPSLRNITIPSITSGLTTTMSQTTTTTTIATTMYNNNQVTSSSDETNNIKENKHYRGVRRRPWGKYAAEIRDPNRKGSRVWLGTFDTAIEAAKAYDKAAFKMRGSKAILNFPLEIGESEESVSSCIKVGVKREREEESKSNNYEKSEFNNNNNSNKHVKKEECSPKAVCPLTPSCWKGFWDTDVMGTIFSVPPLSPLSPLMVV

SEQ ID NO: 31 - DNA, 519 bp, Arabidopsis thaliana
  misc_feature (1)..(519): Ceres GEMINI ID. 5052E2
atgggtcggagaaagatcaagatggagatggttcaggacatgaacacacgacaggttaccttttcaaaacggaggactggtttgttcaagaaggcgagcgagttagccacgctctgcaacgctgagttgggcatcgttgtcttttcaccaggaggcaagcctttctcctacgggaaaccgaatcttgattctgttgcagagcgattcatgagagaatatgatgattcagacagtggcgatgaagaaaaaagtggtaattacaggcctaaactgaagaggctgagtgaacgtctcgatttgctcaaccaagaggttgaagctgagaaggaacgaggcgagaagagtcaggagaagcttgaatctgctggggatgagagattcaaggagtccattgagacgcttaccctcgatgaactcaatgaatacaaagataggcttcagacagtccatggtaggattgaaggtcaagtcaatcacttgcaggcttcgtcttgcctcatgcttctctccagaaaatag

SEQ ID NO: 32 - protein, 172 aa, Arabidopsis thaliana
  misc_feature (1)..(172): Ceres GEMINI ID. 5052E2
MGRRKIKMEMVQDMNTRQVTFSKRRTGLFKKASELATLCNAELGIVVFSPGGKPFSYGKPNLDSVAERFMREYDDSDSGDEEKSGNYRPKLKRLSERLDLLNQEVEAEKERGEKSQEKLESAGDERFKESIETLTLDELNEYKDRLQTVHGRIEGQVNHLQASSCLMLLSRK

SEQ ID NO: 33 - protein, 186 aa, Glycine max
  misc_feature (1)..(186): Ceres CLONE ID no. 681294
MGRRKIEIATLKDPNTRQVTFSKRRTGLFKKANELSILCGAEIAIVVFSIGNKPYSFGHPGVDVIAAKFLQEAANSSDAKQIDAQGNNPSNELGDMNRLNQQLSDVQTQILEEEKKGAEHDERLKQHQVTQLSQYKELQASYLELQHRVKDYVNAIEVSECMILLAQEPVVGITKQMTATKRRKKN

SEQ ID NO: 34 - protein, 248 aa, Zea mays
  misc_feature (1)..(248): Ceres CLONE ID no. 244495
MAPPRRPSMGRQKIEIRRIESDEARQVCFSKRRAGLFKKASELSILCGADVAAVVFSPAGKAFSFGHPSVESVVERFLASSSPSPAGAGAGHSASGGEDRAVSELNRQHGELRAQLDAVKARQERADEAIRKEREAGSPAMAWIDAELSGGAMGHDDLVAFWAALAGVQAAVAASADRLLRDALLVGRRGRQQQQPAQLAGGGGVAFDVGAFGGVGVQVQPPPGFAGVVDLQGFGGQAAAILGAGGPS

SEQ ID NO: 35 - protein, 221 aa, Closterium peracerosum
  misc_feature (1)..(221): Public GI no. 57999638
MGRGKIEIRKIDNATTRQVTFSKRRNGLLKKAYELAVLCDVEIGVIIFSATGKLFQYASTNMDSIVERYRRLALETGKDPRPPWQQQNPPQSTGLGAQHGQHNKHGKEKPGQLQARTQQQRQQEQQEGEAKDTLHGVLTLKKAAEPQPLDLELSRLQAEQSRALVPLDTSAESFEGLGLEEMRQLEKQLEASLSRLRERKEELFNRTISELKSRLEGRSKA

SEQ ID NO: 36 - protein, 148 aa, Brassica napus
  misc_feature (139)..(139): Xaa is any aa, unknown, or other
MEDGEPSSTACLQLKEKLQQNPKTTKGRQKIEIKQISEESKRQVTFSKRRTGLFKKAAELSVLCGAQIGIITFSRRGRIYTFGNADALVENYLRRTPVMLRSHPGGDMANEGEEVDGLKWWEKTVESVPEEEMEEYITXLSGLRDKLW

SEQ ID NO: 37 - protein, 89 aa, Parthenium argentatum
  misc_feature (1)..(89): Ceres CLONE ID no. 1604678
MGRGRVQLKRIENKISRQVTFSKRRTGLLKKAHEISVLCDADVALIVFSTKGKLFEYSTHSSMEAILERYERYSYAEKLLTGLEAETQG

SEQ ID NO: 38 - protein, 95 aa, Brassica oleracea
  misc_feature (1)..(95): Public GI no. 45533872
MGRGRVEMKRIENKINRQVTFSKRRAGLLKKAHEISILCDAEVSLIVFSHKGKLFEYSSESCMEKVLERYERYSYAEKQLKAPDSHVNAQTNWSM

SEQ ID NO: 39 - protein, 95 aa, Brassica oleracea
  misc_feature (1)..(95): Public GI no. 45533888
MGRGRVEMKRIENKINRQVTFSKRRAGLLKKAHEISILCDAEVSLIVFSHKGKLFEYSSESCMEKVLERYERYSYAEKQLKAPDSHVNAQTNWSV

SEQ ID NO: 40 - protein, 95 aa, Brassica oleracea
  misc_feature (1)..(95): Public GI no. 45533884
MGRGRVEMKRIESKINRQVTFSKRRAGLLKKAHEISILCDAEVSLIVFSHKGKLFEYSSESCMEKVLEHYERYSYAEKQLKVPDSHVNAQTNWSV

SEQ ID NO: 41 - protein, 57 aa, Ipomoea nil
  misc_feature (1)..(57): Public GI no. 27372827
MGRGRVQLKRIENKINRQVTFSKRRSGLLKKANEISVLCDADVGVIVFSTKGKLFEY

SEQ ID NO: 42 - protein, 57 aa, Perilla frutescens
  misc_feature (1)..(57): Public GI no. 27372831
MGRGRVQLKRIENKINRQVTFSKRRSGLLKKAHEISILCDADVGLIVFSTKGKLFEY

SEQ ID NO: 43 - protein, 57 aa, Perilla frutescens
  misc_feature (1)..(57): Public GI no. 27372829
MGRGRVQLKRIENKINRQVTFSKRRAGLLKKAHEISVLCDAEVALIVFSHKGKLFEY

SEQ ID NO: 44 - protein, 75 aa, Populus yunnanensis
  misc_feature (1)..(75): Public GI no. 34922009
MEYQNESLESSPLRKLGRGKVEIKRIENTTNRQVTFCKRRSGLLKKAYELSVLCDAEVALIVFSTRGRLYEYSNN

SEQ ID NO: 45 - protein, 75 aa, Populus yunnanensis
  misc_feature (1)..(75): Public GI no. 34922000
MAYQNEPQESSPLRKLGRGKVEIKRIENTTNRQVTFCKRRNGLLKKAYELSVLCDAEVALIVFSSRGRLYEYSNN

SEQ ID NO: 46 - protein, 218 aa, Mimulus lewisii
  misc_feature (1)..(218): Public GI no. 42795299
MARGKIQIKRIENQTNRQVTYSKRRNGLFKKAHELTVLCDAKVSILMISSTQMFDQYQKAVGVDIWNSHYEKMQEHLKKLKDVNRNLRREIRQRVGECLNDLGYHQMVNLSDDIDNSLRIIREKKYKAISNQIDNSKKKLRNVEEINRNLALEFDARQEDPHYGLVENDGDYNALLGFPNGGPRIVALRLPHNHHPHHHHHHLHSGGGSDLTTFALLE

SEQ ID NO: 47 - DNA, 2810 bp, Arabidopsis thaliana
  misc_feature (1)..(2810): Ceres GEMINI ID. 5071B9
atggagatacggagcttgattgtttctatgaaccctaatttatcttcctttgagctctctcgccctgtatctcctctcactcgctcactagttccgttccgatcgactaaactagttccccgctccatttctagggtttcggcgtcgatctccaccccgaatagtgaaactgacaagatctccgttaaacctgtttacgtcccgacgtctcccaatcgcgaactccggactcctcacagtgggtaaattgatccattccattccatttctcttctcttgtttgttttattaagctccaatttcagtttcgtcttttaatttatatgttcttcttacgatcagtgggacttaaaaaattgctcctttaaatgcttcagtatgttttgagtattacaaagttgtaagattttatttttattcatttggtggctcaccattcgacgactacttttgaatttgagtttttgaaaaatgcaatttaacatcagagagtttttttttttatggttgataacttattgtttaacttttgaaaaatgcagataccatttcgatggaacacctcggaagttcttcgagggatggtatttcagggtttccatcccagagaagagggagagtttttgttttatgtattctgtggagaatcctgcatttcggcagagtttgtcaccattggaagtggctctatatggacctagattcactggtgttggagctcagattcttggcgctaatgataaatatttatgccaatacgaacaagactctcacaatttctggggaggtaactccttgacccttaaaatgctgtgtcatgacaataagaaatcatatctgagtcttttctctacttctagtactaatgttcgttattgttgttaaagatctaagtcttatctgaattttgttacattttggttctggtgctttctcaacatgaatttgtatatatgactttaaagattgcttacctaaagtttttactcatgcatagatcgacatgagctagttttggggaatacttttagtgctgtgccaggcgcaaaggctccaaacaaggaggttccaccagaggttctcactcctcccttgttggttactttgttatctgttaaatagttttccaattgtatccggatagtgttctacttctccttgtagaaaatctcaagtttttgttactcttgctattctcttggatgttgatttgtaaagcatgtcgttttattgtaggaatttaacagaagagtgtccgaagggttccaagctactccattttggcatcaaggtcacatttgcgatgatggccggtaattatatgattctatgcacaacaagaattcactatattataaatattggatattgagtatttttgttgaaaatttctgtgtttaaatctgacttgacttgttttgtcagtactgactatgcggaaactgtgaaatctgctcgttgggagtatagtactcgtcccgtttacggttggggtgatgttggggccaaacagaagtcaactgcaggctggcctgcagcttttcctgtatttgagcctcattggcagatatgcatggcaggaggcctttccacaggtgtgagctttgcttgattgacttaaagttaataaatagacggttaagtttacttgcctagtactaacagaaaattaagaaagaaaccaccctctttctatcagcagaaactgctattgtagttcttattttttctcttgtatttgcagggtggatagaatggggcggtgaaaggtttgagtttcgggatgcaccttcttattcagagaagaattggggtggaggcttcccaagaaaatggttttgggtaaaacatttcatccttttgctacatttcttgttgcagactttagttagctagtggacctgtgtatacacccacatgtagtatacttgtttgatagctttatttgtcaatgtctctttacaggtccagtgtaatgtctttgaaggggcaactggagaagttgctttaaccgcaggtggcgggttgaggcaattgcctggattgactgagacctatgaaaatgctgcactggtatgcacttataagatcttcttaagcaatgacagtgagtattagaaggcagatagtttacaaaagctctgggcccttgtaaatctgcaggtttgtgtacactatgatggaaaaatgtacgagtttgttccttggaatggtgttgttagatgggaaatgtctccctggggttattggtatataactgcagagaacgaaaaccatgtggtaaatttgttttactagtttcattcagttttacttttgacatcatatcattcccttatggctagattccaacacccgatgaatgtcttgtgacaggtggaactagaggcaagaacaaatgaagcgggtacacctctgcgtgctcctaccacagaagttgggctagctacggcttgcagagatagttgttacggtgaattgaagttgcagatatgggaacggctatatgatggaagtaaaggcaaggtatgtatgctaatgtgatccaatccctgtagttaaaagtcttaacaaatcctaaggcagtgaaagaagattatgaacgtttgttatggttaacaatgatgcaggtgatattagagacaaagagctcaatggcagcagtggagataggaggaggaccgtggtttgggacatggaaaggagatacgagcaacacgcccgagctactaaaacaggctcttcaggtcccattggatcttgaaagcgccttaggtttggtccctttcttcaagccaccgggtctgtaa

SEQ ID NO: 48 - protein, 488 aa, Arabidopsis thaliana
  misc_feature (1)..(2810): Ceres GEMINI ID. 5071B9
MEIRSLIVSMNPNLSSFELSRPVSPLTRSLVPFRSTKLVPRSISRVSASISTPNSETDKISVKPVYVPTSPNRELRTPHSGYHFDGTPRKFFEGWYFRVSIPEKRESFCFMYSVENPAFRQSLSPLEVALYGPRFTGVGAQILGANDKYLCQYEQDSHNFWGDRHELVLGNTFSAVPGAKAPNKEVPPEEFNRRVSEGFQATPFWHQGHICDDGRTDYAETVKSARWEYSTRPVYGWGDVGAKQKSTAGWPAAFPVFEPHWQICMAGGLSTGWIEWGGERFEFRDAPSYSEKNWGGGFPRKWFWVQCNVFEGATGEVALTAGGGLRQLPGLTETYENAALVCVHYDGKMYEFVPWNGVVRWEMSPWGYWYITAENENHVVELEARTNEAGTPLRAPTTEVGLATACRDSCYGELKLQIWERLYDGSKGKVILETKSSMAAVEIGGGPWFGTWKGDTSNTPELLKQALQVPLDLESALGLVPFFKPPGL

SEQ ID NO: 49 - protein, 490 aa, Brassica napus
  misc_feature (1)..(490): Ceres CLONE ID no. 1109488
MDTRSLAVSMNTNFASFDLSRHLSPLRSAKLSPRSIPRASASISTTNSDSSPSGNAINSEAISVKPVYVPTPPNRELRTPHSGYHFDGTARKFFEGWYFRVSIPEKRESFCFMYSVENPAFRKRLSPLEVGLYGPRFTGVGAQILGANDKYLCQYTEDSHNFWGDRHELVLGNTFSAMPGARSPDKEVPPEEFNRRVSEGFQATPFWHQGHICDDGRTDYAETVKSARWEYSTRPVYGWGDVGTKQKSTAGWPAAFPVFEPHWQICMAGGLSTGWIEWGDERFEFRDAPSYSEKNWGGGFPRKWFWVQCNVFEGAKGEIALTAAGGLRQLPGLTETFENAALVCVHYDGKLYEFVPWNGVVSWEMSPWGYWYMTAENETHMVELEARTNEAGTPLRAPTSEAGLATACKDSCYGELKLQIWERRYDGSKGKVIMEAKSSMAAVEIGGGPWFGTWKGDTSNTPELLKRSLQVPLDLESVFGWVPFFKPPGL

SEQ ID NO: 50 - protein, 515 aa, Eucalyptus gunnii
  misc_feature (1)..(515): Public GI no. 33188419
MEASSVALCEVHRFAPKHGPRALTSPSFGRSRCRSPGRGSLKLGPRRGSGAVVLASASAGDAYGSSTIDRREADSGDKKAASSAPSSPSASPVYVPTPPNRESRTPHSGYHWDGSSRKFFEGWYFKVSIPESRQSFCFMYSVENPAFPKKLSALEVAQYGPRYTGVGAQILGADDKYICQFSEESANFWGSRHELILGNTFMAEKDAKPPNKEVPPQEFNKRVAEGFQVSPLWHQGFIRDDGRSDYVETVKTARWEYSTRPVYGWGNAGSQQKSTAGWLAAFPVFEPHWQICMAGGLSTGWIEWEGERFEFQDAPSYSEKNWGGAFPRKWFWVQCNVFEGAKGEVALTAAGGLRQLPGLTETFENAALIGVHYDGIFYEFVPWNGVVSWEVAPWGSWYMEADNETHKVVVEATTKDRGTTLRAPTVEAGLAPACNDTCFGDLKLQIWERRYDGSKGKLILDVTSNMAAVEIGGGPWFSTWKGKTSTPELLSRALRVPVDVDGFFGLAPFLKPPGL

SEQ ID NO: 51 - DNA, 1677 bp, Gossypium hirsutum
  misc_feature (1)..(1677): Ceres CLONE ID no. 1948913
acaagctttgttcctttaattatttttcttgcaactttcaatggagctaaacacttactctatcaacgagcttcgccatttctcttcttgttatattggtcttcgttctctaaactccaaaaccgccgttaaattatcccaatcttctaacttcaatggactctttcctcgacgcctccgccctctcaggttagggtttcgatcgaactcgccgatcatcgcatgcagctccatcgctgagactgataccgaaacttcttctacggcatccgacagacccgtccccgtcaatccggtttatgttcccacgccggctaatcgagacactcgaactcctcacagcgggtaccactttgatggaaccactcgacaattttttgagggttggtactttaaggtatcaatcccagaacgaaaacagagcttttgcttcatgtactcggtggagaatcctgtatttaggaggaaactgacccaattggaaacactgcagcacggacctagatttacaggagttggggcacaaatccttggtgcttatgacgagtatatatgccaatacagtgatgaatctcagaacttttggggaagcaggcatgagctgatattggggaatacttttttagccaacaaaaactcacgacctccaagtaaggaggtccctcctgaggaattcaatagaaaagttttggagggctttcaagttagcccactttggcatcaaggctttattcgtgacgatggcaggacatcttatgcaagaactgttaaagctgcacgttgggagtacagtactcgccccacctatggatggggtgatgttgggtccaagcagaagtcaacagctggctggcttgcagcttttcccatattcgaaccccattggcaaatttgcatggctggtggactttcaacaggttggatagagtgggatggtgaaaggtttgagtttcaagatgctccttcatactcggaaaagaattggggtggagccttccctagaaaatggttttgggctcaatgcaatgtctttgaaggtgcaagcggagaagttgcattgactgctggtggtggattgaggcaactgcctggcttgactgagacctttgaaaatgctgcattgattggagtgcattatgatgggattttctatgaatttgtgccctggaacggtgttctatcttgggaaattgctccgtggggttactggtgcatcgccgcggagaacgagacacatatggttgagttagaggcaacaacaaatgatccaggtacaacattgcgtgctccaacaatagaggctggtcttgctcctgcatgtaaagacacttgttttggtaatctaagattgcagatctgggaaaaaaaatatggtggcactaaggggaagctaatcttggatgtcaaaagtgacatggcagctctagaagtaggaggagggccatggttcaacacttggaaagggaagaccactacaccagaggtccttaaaactgctcttcaggtgcctgttgatgtggaagggatttttggtttggctccatttttcaaaccccctggactgtgataagagatagatcattcattacattaacaatgcattgactttataaagtatataacaacaatttgtataagaaatcctccttttgattgaaaactgttcttcctttgtgc

SEQ ID NO: 52 - protein, 508 aa, Gossypium hirsutum
  misc_feature (1)..(508): Ceres CLONE ID no. 1948913
MELNTYSINELRHFSSCYIGLRSLNSKTAVKLSQSSNFNGLFPRRLRPLRLGFRSNSPIIACSSIAETDTETSSTASDRPVPVNPVYVPTPANRDTRTPHSGYHFDGTTRQFFEGWYFKVSIPERKQSFCFMYSVENPVFRRKLTQLETLQHGPRFTGVGAQILGAYDEYICQYSDESQNFWGSRHELILGNTFLANKNSRPPSKEVPPEEFNRKVLEGFQVSPLWHQGFIRDDGRTSYARTVKAARWEYSTRPTYGWGDVGSKQKSTAGWLAAFPIFEPHWQICMAGGLSTGWIEWDGERFEFQDAPSYSEKNWGGAFPRKWFWAQCNVFEGASGEVALTAGGGLRQLPGLTETFENAALIGVHYDGIFYEFVPWNGVLSWEIAPWGYWCIAAENETHMVELEATTNDPGTTLRAPTIEAGLAPACKDTCFGNLRLQIWEKKYGGTKGKLILDVKSDMAALEVGGGPWFNTWKGKTTTPEVLKTALQVPVDVEGIFGLAPFFKPPGL

SEQ ID NO: 53 - protein, 483 aa, Helianthus annuus
  misc_feature (1)..(483): Public GI no. 80971684
MELQTTTTSPLFSSLLASSKPNNVKSSVRLKHERNRRLSAAKTDVYGVELQSQEIVNPVYVPTPTNRPLRPPHSGYHFDGTTRKFFEGWYFKVSIPEQRQSFCFMYSVENPAFKKDLNILEQLQHGPRFTGVGAQILGAHDKYICQYSKESHNFWGSRHELMLGNSFSVQTGKQPPNSEVPPQVFNQRVIEGFQVTPLWHQGFIRDDGRTSYAETVKTARWEYSTRPVYGWGDVGSKQKSTAGWLAAFPVFEPHWQICMAGGLSTGWIEWGDERYEFEDAPSYCEKNWGGGFPRKWFWVQCNVFKGASGEVGLTCGGGLRQLPGLNETFENAALIGVHHGGIFYEFVPWNGVVEWEVAEWGYWHVTAQNETHKVELEASTKDPGTTLRAPTTEAGLAPACKDTCFAHLTLKLWEKGSAAAAADGKLILDVTSNMAAVEVGGGPWFNTWKGKTYTPEVINRALNLPVDVDGILGSFPLLKPPGL

SEQ ID NO: 54 - protein, 479 aa, Glycine max
  misc_feature (1)..(479): Ceres CLONE ID no. 1245537
MEAKLWNWNPLLLPPRFSSLKHAFPSTTTRLVAHNSVSETVPIHKEKEQTLPSVKPTYSPTPPNRGLRTPHSGYHFDGTTRKFFEGWYFKVSIPERKQSFCFMYTVESPLFRKPLTQLELAQYGPRFTGVGAQILGADDKYVCQHSPQSHFFWGSRHELMLGNTFEPNQNSKPPNKEVPPQEFNDRVLEGFQVTPLWHQGFIRDDGRSNYVETVKTARWEYSTRPVYGWGDVGSTQKSTAGWLAAFPVFEPHWQICMAGGLSTGWIEWDGERIEFDNAPSYSEKNWGGGFPRKWFWVQCNVFEGASGEIALTAAGGLRQIPGITETFENAALIGIHYGGNFYEFVPWNGVVNWEVTTWGYWFMSADNGKYVVELEATTEDPGTTLRAPTAEAGFAPACKDTCFGNLKLQMWERRYDGSKGKIILDVSSNMAALEVGGGPWFNTWKGKTSTPAALSRVLELPIDVEGIFNPVPLFKPPGL

SEQ ID NO: 55 - protein, 483 aa, Helianthus annuus
  misc_feature (1)..(483): Public GI no. 80971690
MELQTTTTSPLFSSLLASSKPNNVKSSVRLKLERNRRLSAAKTDVYGVELQSQEIVNPVYVPTPTNRPLRPPHSGYHFDGTTRKFFEGWYFKVSIPEQRQSFCFMYSVENPAFKKDLNILEQLQHGPRFTGVGAQILGAHDKYICQYSKESHNFWGSRHELMLGNSFSVQTGKQPPNSEVPPQVFNQRVIEGFQVTPLWHQGFIRDDGRTSYAETVKTARWEYSTRPVYGWGDVGSKQKSTAGWLAAFPVFEPHWQICMAGGLSTGWIEWGDERYEFENAPSYCEKNWGGGFPRKWFWVQCNVFKGASGEVGLTCGGGLRQLPGLNETFENAALIGVHHGGIFYEFVPWNGVVEWEVAEWGYWHVTAQNETHKVELEASTKDPGTTLRAPTTEAGLAPACKDTCFAHLTLKLWEKGSAAAAADGKLILDVTSNMAAVEVGGGPWFNTWKGKTYTPEVINRALNLPVDVDGILGSFPLLKPPGL

SEQ ID NO: 56 - DNA, 1506 bp, Populus balsamifera subsp. trichocarpa
  misc_feature (1)..(1506): Ceres ANNOT ID no. 1530974
atggaagtacacattcacaatttctcccctaatttctcaccacttcgacccctaacaaaccctaattcctctatcaaactctaccactctcgagattctaccttttctataactctatcgaaatcactcaagctagggttccgatcatcgccactgctggcggcgtccactcccatcactaacaccgatacttcactagcctcaaatggagaaaatgagaggagtgtgagtccagtttatgtgccaacgccacctaatcgtgggctccgtactcctcacagcgggtaccattatgatgggacgacgcggaacttttttgaaggttggtattttaaagtgtcgataccggagcggaaacagaatttctgtttcatgtattcagtggagaatcctgcgtttcctaagaaattgactgccttggagtcggcacagcatggatcgagatcaattggagttggtgctcagattctaggggcttatgacaagtatatttgccaattctctgaagaatcgcaagatttctggggaagtaggcatgagctggcattggggaatacttttgttgctgagagaaatatgcggcctccaactaaagaggttcctcctcaggaatttgataaaagagttttagaaggatttcaagtaagcccactttggcaccaaggctttattcgtgatgatggcaggtcagattatgtggaaactgtgaaaactgcacgttggcagtacagtacacggcctgtttatggatggggtaatgttgggtctacacagaagtcgactgcagggtggcttgctgcttttcctgtatttgaaccccactggcaaatatgcatggccggaggactttcaacaggatggatagagtgggatggtgaaagatttgagttcgaagatgctccttcttattctgaaaagaactggggtggagccttcccaagaaaatggttttgggttcaatgtaatgtctttgaaggtgcaagtggagaagttgctttgaccgcagctggtgggttgaggcaaatacctggaccaagtgagacttttgaaaatgctgcattggttggagtacattatgatgggattttctatgaatttgtgccatggaatggtgttgtaaattgggaaatttctccatggggttactggtacatggctgcagacaatgggacacatttggttgaattggaggcaacaacaaaggaattgggaacagcattgcgtgctccaacagcagaagctgggctttctccagcttgtaaagataccggttttagttttctgaaattgcaaatatgggaacgtcgatatgatggcactaaggggaagttaattttggatgttacaagcgacatggcattagtagaagttggaggaggaccatggtttaccacttggaaaggcaagacatctatggcagagcttgtcagccgcactattggagcccccattgacgtggatggaatttttagttttgttcctctgttcaagccccctggtttataa

SEQ ID NO: 57 - protein, 501 aa, Populus balsamifera subsp. trichocarpa
  misc_feature (1)..(501): Ceres ANNOT ID no. 1530974
MEVHIHNFSPNFSPLRPLTNPNSSIKLYHSRDSTFSITLSKSLKLGFRSSPLLAASTPITNTDTSLASNGENERSVSPVYVPTPPNRGLRTPHSGYHYDGTTRNFFEGWYFKVSIPERKQNFCFMYSVENPAFPKKLTALESAQHGSRSIGVGAQILGAYDKYICQFSEESQDFWGSRHELALGNTFVAERNMRPPTKEVPPQEFDKRVLEGFQVSPLWHQGFIRDDGRSDYVETVKTARWQYSTRPVYGWGNVGSTQKSTAGWLAAFPVFEPHWQICMAGGLSTGWIEWDGERFEFEDAPSYSEKNWGGAFPRKWFWVQCNVFEGASGEVALTAAGGLRQIPGPSETFENAALVGVHYDGIFYEFVPWNGVVNWEISPWGYWYMAADNGTHLVELEATTKELGTALRAPTAEAGLSPACKDTGFSFLKLQIWERRYDGTKGKLILDVTSDMALVEVGGGPWFTTWKGKTSMAELVSRTIGAPIDVDGIFSFVPLFKPPGL

SEQ ID NO: 58 - protein, 480 aa, Glycine max
  misc_feature (1)..(480): Ceres CLONE ID no. 574132
MEAKLWESPLLSPSLPLLPSLNLHFPSSTRLLARSFFSDTVPIDNKEKEQLVTSVKPTYSPTPPNRHLRTPHSGYHFDGTTRKFFEGWYFKLSIPERRQSFCFMYSVESPSFRKPLTPLEVAQYGSRFTGVGAQILGADDKYICQFSPESQFFWGSRHELMLGNTFVPNQNSKPPNKEVPPQEFNDRVLEGFQVSPLWHQGFIRDDGRSNYVETVKTARWEYSTRPVYGWGDVGSTQKSTAGWLAAFPVFEPHWQICMAGGLSTGWIEWDGKRIEFDNAPSYSEKNWGGGFPRKWFWVQCNVFEGASGEIALTAAGGLRQIPGIAETFENAALIGIHYGGIFYEFVPWNGVVNWEVTTWGYWFMSADNGRYVVEIEATTEDPGTTLRAPTAEAGLAPACKDTCFGDLKLQMWERRYDGSKGKIILDVSSNMAALEVGGGPWFNTWKGKTSTPAALSRVLELPIDVEGIFNPVPLFKPPGL

SEQ ID NO: 59 - protein, 501 aa, Solanum tuberosum
  misc_feature (1)..(501): Public GI no. 47078321
MESFYSVSAISPISKNVGFSRIRTEFATSIANGELFLNNYSSTILKVQSQKSRHAFVVKADSSVDTTKKENREPVKPLYSSTPSNRPLRTPHSGYHFDGSTRKFFEGWFFKVSIPECRQSFCFMYSVESPSFTKKLSSFEELQYGPRFTGVGAQILGADDKYICQYSEESSNFWGSRHELMLGNTFVAQNSAKPPNKEVRPQEFNHRVTEGFQVTPLWHQGSIRDDGRTDYTEIVKTASWEYSTRPIYGWGDVNSKQKSTAGWPAAFPVFEPHWQVCMAAGLSTGWIEWDGQRFEFQNAPSYSEKNWGGSFPRKWFWVQCSVFEGAIGDVALTAGGGLRRLPGLNETFESVALIGIHYGGIFYEFVPWNASVSWEITPWGKWHISAENETHMVLLEATTEDPGTTLRAPTEEMGLAPACRDTCFGELRLQLWERKSNGSKGEVILDVTSNMAGLEVGGGPWFNTWEGKAEMPEIVTRAINVPVDLDGIFSCVPSLLKPPGL

SEQ ID NO: 60 - protein, 470 aa, Oryza sativa subsp. japonica
  misc_feature (1)..(470): Public GI no. 50906901
MDLAAAAVAVSFPRPAPPPRRCAPRRHRRALAPRAASSSPSPSTAVAAPVYAPTPRDRALRTPHSGYHYDGTARPFFEGWYFKVSIPECRQSFCFMYSVENPLFRDGMSDLDRVIHGSRFTGVGAQILGADDKYICQFTEKSNNFWGSRHELMLGNTFIPNNGSTPPEGEVPPQEFSSRVLEGFQVTPIWHQGFIRDDGRSKYVPNVQTARWEYSTRPVYGWGDVTSKQKSTAGWLAAFPFFEPHWQICMAGGLSTGWIEWDGERFEFENAPSYSEKNWGAGFPRKWYWVQCNVFSGASGEVALTAAGGLRKIGLGETYESPSLIGIHYEGKFYEFVPWTGTVSWDIAPWGHWKLSGENKNHLVEIEATTKEPGTALRAPTMEAGLVPACKDTCYGDLRLQMWEKRNDGGKGKMILDATSNMAALEVGGGPWFNGWKGTTVSNEIVNNVVGTQVDVESLFPIPFLKPPGL

SEQ ID NO: 61 - protein, 467 aa, Triticum aestivum
  misc_feature (1)..(467): Ceres CLONE ID no. 754013
MNVAGPATAFVPSARPAPRAPRPLWRPSARCNPSPRAATEPPAAAPVYAPTPRDRPLRTPHSGYHYDGTARAFFEGWYFKVSIPECRQSFCFMYSVENPFFRDGMTALDRTLYGPQFTGVGAQILGADDKYICQFSEKSNNFWGSRHELILGNTFIPNKGSTPPEREIPPQEFSNRVLEGYQVTPTWHQGFIRDDGRSKYVPNVQTARWEYSTRPVYGWGDVTSKQKSTAGWLAAFPFFEPHWQICMAGGLSTGWIEWDGERFEFENAPSYSEKNWGGGFPRKWYWIQCNAFSGTSGEVALTAAGGLRKIGLGDTYESPSLIGVHHEGKFYEFVPWTGTVSWDIAPWGHWRMSGENKNHLVEIEATTKEPGTALRAPTMEAGLVPACKDTCYGDLKLQMWEKKYDGSKGKIILDTTSNMAAVEVGGGPWFNGWKGTTASPELVNNIVGTQIDVESLFPIPFLKPPGL

SEQ ID NO: 62 - protein, 467 aa, Triticum aestivum
  misc_feature (1)..(467): Public GI no. 91694297
MNVAGPATAFVPSARPAPRAPRPLWRPSARCNPSPRAATEPPAAAPVYAPTPRDRPLRTPHSGYHYDGTARAFFEGWYFKVSIPECRQSFCFMYSVENPFFRDGMTALDRTLYGPQFTGVGAQILGADDKYICQFSEKSNNFWGSRHELILGNTFIPNKGSTPPEREIPPQEFSNRVLEGYQVTPTWHQGFIRDDGRSKYVPNVQTARWEYSTRPVYGWGDVTSKQKSTAGWLAAFPFFEPHWQICMAGGLSTGWIEWDGERFEFENAPSYSEKNWGGGFPRKWYWIQCNVFSGTSGEVALTAAGGLRKIGLGDTYESPSLIGIHHEGKFYEFVPWTGTVSWDIAPWGHWKMSGENKNHLVEIEATTKEPGTALRAPTIEAGLVPACKDTCYGDLKLQMWEKKYDGSKGKIILDTTSNMAALEVGGGPWFNGWKGTTATNEPVNNIVGTQIDVESLFPIPFLKPPGL

SEQ ID NO: 63 - DNA, 2557 bp, Arabidopsis thaliana
  misc_feature (1)..(2557): Ceres ANNOT ID 552252
atggagtctctgctctctagttcttctcttgtttccgctggtaaatctcgtccttttctggtttcaggttttatttgttgtttaggtttcgtttttgtgattcagaaccatacaaaaagtttgaacttttctgaatataaaataaggaaaaagtttcgatttttataatgaattgtttactagatcgaagtaggtgacaaaggttattgtgtggagaagcataatttctgggcttgactttgaattttgtttctcatgcatgcaacttatcaatcagctggtgggttttgttggaagaagcagaatctaaagctccactctttatcaggttcgttagggttttatgggtttttgaaattaaatactcaatcatcttagtctcattattctattggttgaatcacattttctaatttggaatttatgagacaatgtatgttggacttagttgaagttcttctctttggttatagttgaagtgttactgatgttgtttagctctttacaccaatatatacacccaattttgcagaaatccgagttctgcgttgtgattcgagtaaagttgtcgcaaaaccgaagtttaggaacaatcttgttaggcctgatggtcaaggatcttcattgttgttgtatccaaaacataagtcgagatttcgggttaatgccactgcgggtcagcctgaggctttcgactcgaatagcaaacagaagtcttttagagactcgttagatgcgttttacaggttttctaggcctcatacagttattggcacagttaagtttctctttaaaaatgtaactcttttaaaacgcaatctttcagggttttcaaggagataacattagctctgtgattggatttgcaggtgcttagcattttatctgtatctttcttagcagtagagaaggtttctgatatatctcctttacttttcactggcatcttggaggtaatgaatatataacacataatgaccgatgaagaagatacatttttttcgtctctctgtttaaacaattgggttttgttttcaggctgttgttgcagctctcatgatgaacatttacatagttgggctaaatcagttgtctgatgttgaaatagataaggtaacatgcaaattttcttcatatgagttcgagagactgatgagattaatagcagctagtgcctagatcatctctatgtgggtttttgcaggttaacaagccctatcttccattggcatcaggagaatattctgttaacaccggcattgcaatagtagcttccttctccatcatggtatggtgccattttcacaaaatttcaacttttagaattctataagttactgaaatagtttgttataaatcgttatagagtttctggcttgggtggattgttggttcatggccattgttctgggctctttttgtgagtttcatgctcggtactgcatactctatcaatgtaagtaagtttctcaatactagaatttggctcaaatcaaaatctgcagtttctagttttaggttaatgaggttttaataacttacttctactacaaacagttgccacttttacggtggaaaagatttgcattggttgcagcaatgtgtatcctcgctgtccgagctattattgttcaaatcgccttttatctacatattcaggtactaaaccattttccttatgttttgtagttgttttcatcaaaatcacttttatattactaaagctgtgaaactttgttgcagacacatgtgtttggaagaccaatcttgttcactaggcctcttattttcgccactgcgtttatgagctttttctctgtcgttattgcattgtttaaggtaaacaaagatggaaaaagattaaatctatgtatacttaaagtaaagcattctactgttattgatgagaagttttcttttttggttggatgcaggatatacctgatatcgaaggggataagatattcggaatccgatcattctctgtaactctgggtcagaaacgggtacgatatctaaactaaagaaattgttttgactcaagtgttggattaagattacagaagaaagaaaactgtttttgtttcttgcaaaattcaggtgttttggacatgtgttacactacttcaaatggcttacgctgttgcaattctagttggagccacatctccattcatatggagcaaagtcatctcggtaacaatctttctttacccatcgaaaactcgctaattcatcgtttgagtggtactggtttcattttgttccgttctgttgattttttttcaggttgtgggtcatgttatactcgcaacaactttgtgggctcgagctaagtccgttgatctgagtagcaaaaccgaaataacttcatgttatatgttcatatggaaggttagattcgtttataaatagagtctttactgcctttttatgcgctccaatttggaattaaaatagcctttcagtttcatcgaatcaccattatactgataaattctcatttctgcatcagctcttttatgcagagtacttgctgttaccttttttgaagtga

SEQ ID NO: 64 - protein, 393 aa, Arabidopsis thaliana
  misc_feature (1)..(393): Ceres ANNOT ID 552252
MESLLSSSSLVSAAGGFCWKKQNLKLHSLSEIRVLRCDSSKVVAKPKFRNNLVRPDGQGSSLLLYPKHKSRFRVNATAGQPEAFDSNSKQKSFRDSLDAFYRFSRPHTVIGTVLSILSVSFLAVEKVSDISPLLFTGILEAVVAALMMNIYIVGLNQLSDVEIDKVNKPYLPLASGEYSVNTGIAIVASFSIMSFWLGWIVGSWPLFWALFVSFMLGTAYSINLPLLRWKRFALVAAMCILAVRAIIVQIAFYLHIQTHVFGRPILFTRPLIFATAFMSFFSVVIALFKDIPDIEGDKIFGIRSFSVTLGQKRVFWTCVTLLQMAYAVAILVGATSPFIWSKVISVVGHVILATTLWARAKSVDLSSKTEITSCYMFIWKLFYAEYLLLPFLK

SEQ ID NO: 65 - protein, 395 aa, Glycine max
  misc_feature (1)..(395): Public GI no. 81295666
MDSMLLRSFPNINNASSLATTGSYLPNASWHNRKIQKEYNFLRFRWPSLNHHYKSIEGGCTCKKCNIKFVVKATSEKSFESEPQAFDPKSILDSVSLLAVEKISDISPLFFTGVLEAVVAALFMNIYIVGLNQLSDVEIDKINKPYLPLASGEYVKNSLDAFYRFSRPHTVIGTALSIISSFETGVTIVASFSILSFWLGWVVGSWPLFWALFVSFVLGTAYSINVPLLRWKRFAVLAAMCILAVRAVIVQLAFFLHIQTHVYKRPPVFSRSLIFATAFMSFFSVVIALFKDIPDIEGDKVFGIQSFSVRLGQKPVFWTCVILLEIAYGVALLVGAASPCLWSKIVTGLGHAVLASILWFHAKSVDLKSKASITSFYMFIWKLFYAEYLLIPFVR

SEQ ID NO: 66 - protein, 411 aa, Medicago sativa
  misc_feature (1)..(411): Public GI no. 51949754
MDSLLVGSFPKSCSYSFTTTGGNLWRSKRYVNNCYSIPSSCASKASRHKKIQNEYNILRFWQPSLNHYCEGAEGRSTYQECNKKFVVKAAPEQSFESEHPAFDPKNILDTVKNSLDAFYRFSRPHTVIGTALSIISVSLLAVEKLSDISPLFFTGVLEAVVAALFMNIYIVGLNQLSDVEIDKINKPYLPLASGEYSFATGAIIVVSSSILSFWLAWIVGSWPLFWALFISFVLGTAYSINVPLLRWKRFAVLAAMCILSVRAVIVQLAFFLHMQTFVYKRPIVFSRPLIFATAFMSFFSVVIALFKDIPDIEGDKIFGIQSFSVRLGQKRVFWICVTLLELAYGVSLVVGATSSCLWSKIVTSLGHAVLASILFNHAKSVDLKSKASITSFYMFIWKLFYAEYFLIPLVR

SEQ ID NO: 67 - protein, 411 aa, Medicago truncatula
  misc_feature (1)..(411): Public GI no. 92882118
MDSLLAGSFPKSFSYSFTTTGGNLWRSKRCVNNYYSIPSSCASKASRHKKIQNEYNTLRFWQPSLNHYCKGAEGRSTYQECNGKFVVKAAPEQSFESEHPAFDPKNILVAVKNSLDAFYRFSRPHTVIGTALSIISVSLLAAEKLSDISPLFFTGVLEAVVAALFMNIYIVGLNQLSDVEIDKINKPYLPLASGEYSFATGAIIVVSSSILSFWLAWIVGSWPLFWALFISFVLGTAYSINVPLLRWKRFAVLAAMCILSVRAVIVQLAFFLHMQTFVYKRPVVFSRPLIFATAFMSFFSVVIALFKDIPDIEGDKIFGIQSFSVRLGQKRVFWICVTLLELAYGVSLVVGATSSCLWSKIVTSLGHAVLASILFNHAKSVDLKSKASITSFYMFIWKLFYAEYFLIPLVR

SEQ ID NO: 68 - protein, 411 aa, Glycine max
  misc_feature (1)..(411): Public GI no. 61808320
MDSLLLRSFPNINNASSLTTTGANFSRTKSFANIYHASSYLPNASWHNRKIQKEYNFLRFRWPSLNHHYKSIEGGCTCKKCNIKFVVKATSEKSFESEPQAFDPKSILDSVKNSLDAFYRFSRPHTVIGTALSIISVSLLAVDKISDISPLFFTGVLEAVVAALFMNIYIVGSNQLFDVEIYKINKPYLPLASGEYSFETGVTIDASFSILSFWLGWVVGSWPLFWALFEIFVLGTAYSINVPLLRWKRFAVLAAMCILAVRAVIVQLAFFLHIQTHVYKRPPVFSRSLIFATAFMSFFSVVIALFKDIPDIEGDKVFGIQSFSVRLSQKPVFWTCVTLLEIAYGVALLVGAASPCLWSKIFTGLGHAVLASILWFHAKSVDLKSKASITSFYMFIWKLFYAEYLLIPFVR

SEQ ID NO: 69 - protein, 402 aa, Oryza sativa subsp. japonica
  misc_feature (1)..(402): Public GI no. 51536170
MDSLRLRPSLLAARAPGAASLPPLRRDHFLPPLCSIHRNGKRPVSLSSQRTQGPSFDQCQKFFGWKSSHHRIPHRPTSSSADASGQPLQSSAEAHDSSSIWKPISSSLDAFYALSIVSVSLLAVENLSDVSPLFLTGLLEICFVTLVQAVVAALFMNIYIVGLNQLFDIEIDKVNKPTLPLASGEYSPATGVALVSAFAAMSFGLGWAVGSQPLFLALFISFILGTAYSINLPFLRWKRSAVVAALCILAVRAVIVQLAFFLHIQATFVFRRPAVFTRPLIFATAFMTFFSVVIALFKDIPDIEGDRIFGIKSFSVRLGQKKVFWICVGLLEMAYCVAILMGATSACLWSKYATVVGHAILAAILWNRSRSIDLTSKTAITSFYMFIWKLFYAEYLLIPLVR

SEQ ID NO: 70 - DNA, 1735 bp, Panicum virgatum
  misc_feature (1)..(1735): Ceres CLONE ID no. 1789748
ctattgatccaccaccttccgtcctctctccagccccgtgatctcgtggccgtgcacgcctgcagcgctctcgaagcgagcggggccgcgccgcccggtcgcccaatcgctgccggcgacggcgaccgccgcggcaacaggagtgggcacgggcggcagccggcaggacaacgagaggggcactgtagcctgaaaaggactgcggggaaacggccgtgacctggcaactccaagggtccatccaagccgcgcccgcccgccgcaacgcgaagtccccacgcctcgcctctccagcctccgctcgctcgctctcggcttccctcgggccggccgcggcgccaagggatggacgcgctccgcctccggccgtccctcctctccgcgcggcccggcgccgcccgcccgcgagatcattttttaccaccattttgttcaattcagcgaaatggggaaccgcgagtgtgtttttccgcccaaaggacccaaggcccttccttgtatcactgtcagaaatccttcgattggaaatccacctattctaggatatcacgtcagtcaacaagtacttctattaatgcttcgggccaaccgctgcaatctgaacctgaagcacatgattctgcaagcatctggagggcaatatcatcttcattagatgtgttttacagattttcccggccacatactgtcatcggaacagcgttaagcatagtctcagtttcccttctagctgtccagagcttgtctgatatatctcctttgttcctcactggtttgctggaggcagtggtagcagctcttttcatgaacatttatattgttggactgaaccagttattcgacattgagatagacaaggtcaacaagccaactcttccactggcatctggggaatatacccctacaactgggattgcaatagtatcggtctttgctgctatgagctttggccttggatgggctgttggatcacaacctctgttttgggctcttttcataagcttcgttcttggaactgcatactcaatcaatttgccatacttaagatggaagagatctgctgttgttgcagcactctgcatattagcagttcgtgcagtgattgttcagctggccttttttctccacattcagacctttgttttcagaagaccagcagtttttacgaggccattgatgtttgcaactggatttatgacgttcttctcagttgtaatagcacttttcaaggatatacctgatattgaaggggaccgcatatttgggattcgatcattcagtgttcgtttgggccaaaagaaggtcttctggatctgtgttggcctgcttgagatggcttacggcgttgcaatactgatgggagcaacttctacctctctgtggagcaaaaccgcaacaattgccgggcattccatcctcgcggcgatcctatggagctgcgcgcggtcggtcgacctgacgagcaaggccgcgataacatccttctacatgttcatctggaagctgttctacgcggagtacctgctcatccccctggtgcgttgactccccggcgatggcgacggcgacggcgacggcgggacaacaactccacgggaggactttgagcgccggagtagtaaacgcccgtttgaagccggacgccacatgcgcgcgcacgagggggagaggatggattggcatctggcaggctacggccggcgcccaataaaacggaataaaattcccttcccct

SEQ ID NO: 71 - protein, 399 aa, Panicum virgatum
  misc_feature (1)..(399): Ceres CLONE ID no. 1789748
MDALRLRPSLLSARPGAARPRDHFLPPFCSIQRNGEPRVCFSAQRTQGPSLYHCQKSFDWKSTYSRISRQSTSTSINASGQPLQSEPEAHDSASIWRAISSSLDVFYRFSRPHTVIGTALSIVSVSLLAVQSLSDISPLFLTGLLEAVVAALFMNIYIVGLNQLFDIEIDKVNKPTLPLASGEYTPTTGIAIVSVFAAMSFGLGWAVGSQPLFWALFISFVLGTAYSINLPYLRWKRSAVVAALCILAVRAVIVQLAFFLHIQTFVFRRPAVFTRPLMFATGFMTFFSVVIALFKDIPDIEGDRIFGIRSFSVRLGQKKVFWICVGLLEMAYGVAILMGATSTSLWSKTATIAGHSILAAILWSCARSVDLTSKAAITSFYMFIWKLFYAEYLLIPLVR

SEQ ID NO: 72 - protein, 399 aa, Zea mays
  misc_feature (1)..(399): Ceres CLONE ID no. 395119
MDALRLRPSLLPVRPGAARPRDHFLPPCCSIQRNGEGRICFSSQRTQGPTLHHHQKFFEWKSSYCRISHRSLNTSVNASGQQLQSEPETHDSTTIWRAISSSLDAFYRFSRPHTVIGTALSIVSVSLLAVQSLSDISPLFLTGLLEAVVAALFMNIYIVGLNQLFDIEIDKVNKPTLPLASGEYTLATGVAIVSVFAAMSFGLGWAVGSQPLFWALFISFVLGTAYSINLPYLRWKRFAVVAALCILAVRAVIVQLAFFLHIQTFVFRRPAVFSRPLLFATGFMTFFSVVIALFKDIPDIEGDRIFGIRSFSVRLGQKKVFWICVGLLEMAYSVAILMGATSSCLWSKTATIAGHSILAAILWSCARSVDLTSKAAITSFYMFIWKLFYAEYLLIPLVR

SEQ ID NO: 73 - protein, 399 aa, Zea mays
  misc_feature (1)..(399): Public GI no. 81295658
MDALRLRPSLLSVRPGAARPRDHFLPPCCSIQRNGEGRICFSSQRTQGPTLHHHQKFFEWKSSYCRISHRSLNTSVNASGQQLQSEPETHDSTTIWRAISSSLDAFYRFSRPHTVIGTALSIVSVSLLAVQSLSDISPLFLTGLLEAVVAALFMNIYIVGLNQLFDIEIDKVNKPTLPLASGEYTPATGVAIVSVFAAMSFGLGWAVGSQPLFWALFISFVLGTAYSINLPYLRWKRFAVVAALCILAVRAVIVQLAFFLHIQTFVFRRPAVFSRPLLFATGFMTFFSVVIALFKDIPDIEGDRIFGIRSFSVRLGQKKVFWICVGLLEMAYSVAILMGATSSCLWSKTATIAGHSILAAILWSCARSVDLTSKAAITSFYMFIWKLFYAEYLLIPLVR

SEQ ID NO: 74 - DNA, 1164 bp, Populus balsamifera subsp. trichocarpa
  misc_feature (1)..(1164): Ceres ANNOT ID no. 1478147
atggagtctttgcttcctgggtcctttcctcagctttcctctccacttccttctggtgggaattacttgggcgagaccaaagtcaaagtgggtcattctccaagagaagttcaagtttacaggtgcagagcaaggaagatcctagaaagacagtgtgttgttaggtttcagcggcatcatagtggattttctgtgaaaaaatctacaatttaccaggaaaataatgctaagttcttagtgcatgctgcctctggacaaccttttgaatctgagtctggagcttataatcctgagagtacttcaaaatctgtcaaaaatgcattagatgctttctacagattttcacggcctcacactgttataggcacagctttgagcatcttatcagtttctctccttgcaattgagaagctctcggatatttctccactatttttcacaggggtgttggaggctgtggctgctgctctcatgatgaatatttatattgtgggtttaaatcagttgactgacattgaaatagacaaggttaacaagccatatcttccattggcgtcgggggagtattccataagtactggtgtgatgattgttacatctttctctatcatgttgcagaacgggggggtggggattgattatgtgccttcatctgcaaaccagtttttcaaaattgtagtgagtttttggcttggatgggtcgtaggttcgtggccattattttgggctcttttcatcagttttgttcttggaacagcatattcaatcaatctgccactgttgagatggaaaaggtttgcgttcgttgcagcagtgtgcatcctagctgttagggcagtgattgttcaacttgccttttatctgcatatgcagacccatgtgtatggaagaccacctgtcctttcaagacctctaatttttgcaactgcatttatgagcttcttctcagttgttatagcactattcaaggacatacctgatattgaaggagataagatttttggcattcggtcttttacagtgcgtttgggacaaaaccgggtattttggacctgcatttcattacttgaaattgcttatgctgttgccattttggttggagcagcatcttcctatacttggagcaaatatatcacggtagtgctcttgcttctttatttataa

SEQ ID NO: 75 - protein, 387 aa, Populus balsamifera subsp. trichocarpa
  misc_feature (1)..(387): Ceres ANNOT ID no. 1478147
MESLLPGSFPQLSSPLPSGGNYLGETKVKVGHSPREVQVYRCRARKILERQCVVRFQRHHSGFSVKKSTIYQENNAKFLVHAASGQPFESESGAYNPESTSKSVKNALDAFYRFSRPHTVIGTALSILSVSLLAIEKLSDISPLFFTGVLEAVAAALMMNIYIVGLNQLTDIEIDKVNKPYLPLASGEYSISTGVMIVTSFSIMLQNGGVGIDYVPSSANQFFKIVVSFWLGWVVGSWPLFWALFISFVLGTAYSINLPLLRWKRFAFVAAVCILAVRAVIVQLAFYLHMQTHVYGRPPVLSRPLIFATAFMSFFSVVIALFKDIPDIEGDKIFGIRSFTVRLGQNRVFWTCISLLEIAYAVAILVGAASSYTWSKYITVVLLLLYL

SEQ ID NO: 76 - DNA, 672 bp, Arabidopsis thaliana
  misc_feature (1)..(672): Ceres ANNOT ID no. 859061
aattctacagttctcatgctaaaccatattttttgctctctgttccttcaaaatcatttctttctcttctttgattcccaaagatcacttctttgtctttgatttttgattttttttctctctggcgtgaaggaagaagctttatttcatggagtctctgctctctagttcttctcttgtttccgctgctggtgggttttgttggaagaagcagaatctaaagctccactctttatcagaaatccgagttctgcgttgtgattcgagtaaagttgtcgcaaaaccgaagtttaggaacaatcttgttaggcctgatggtcaaggatcttcattgttgttgtatccaaaacataagtcgagatttcgggttaatgccactgcgggtcagcctgaggctttcgactcgaatagcaaacagaagtcttttagagactcgttagatgcgttttacaggttttctaggcctcatacagttattggcacagtgcttagcattttatctgtatctttcttagcagtagagaaggtttctgatatatctcctttacttttcactggcatcttggaggctgttgttgcagctctcatgatgaacatttacatagttgggctaaatcagttgtctgatgttgaaatagataaggttaacaagccctatcttccattggcatc

SEQ ID NO: 77 - protein, 174 aa, Arabidopsis thaliana
  misc_feature (1)..(672): Ceres ANNOT ID no. 859061
MESLLSSSSLVSAAGGFCWKKQNLKLHSLSEIRVLRCDSSKVVAKPKFRNNLVRPDGQGSSLLLYPKHKSRFRVNATAGQPEAFDSNSKQKSFRDSLDAFYRFSRPHTVIGTVLSILSVSFLAVEKVSDISPLLFTGILEAVVAALMMNIYIVGLNQLSDVEIDKVNKPYLPLA

SEQ ID NO: 78 - protein, 176 aa, Artificial Sequence
  misc_feature (1)..(176): Public GI no. 81295666_T
MDSMLLRSFPNINNASSLATTGSYLPNASWHNRKIQKEYNFLRFRWPSLNHHYKSIEGGCTCKKCNIKFVVKATSEKSFESEPQAFDPKSILDSVKNSLDAFYRFSRPHTVIGTALSIISVSLLAVEKISDISPLFFTGVLEAVVAALFMNIYIVGLNQLSDVEIDKINKPYLPLA

SEQ ID NO: 79 - protein, 192 aa, Artificial Sequence
  misc_feature (1)..(192): Public GI no. 51949754_T
MDSLLVGSFPKSCSYSFTTTGGNLWRSKRYVNNCYSIPSSCASKASRHKKIQNEYNILRFWQPSLNHYCEGAEGRSTYQECNKKFVVKAAPEQSFESEHPAFDPKNILDTVKNSLDAFYRFSRPHTVIGTALSIISVSLLAVEKLSDISPLFFTGVLEAVVAALFMNIYIVGLNQLSDVEIDKINKPYLPLA

SEQ ID NO: 80 - protein, 192 aa, Artificial Sequence
  misc_feature (1)..(192): Public GI no. 92882118_T
MDSLLAGSFPKSFSYSFTTTGGNLWRSKRCVNNYYSIPSSCASKASRHKKIQNEYNTLRFWQPSLNHYCKGAEGRSTYQECNGKFVVKAAPEQSFESEHPAFDPKNILVAVKNSLDAFYRFSRPHTVIGTALSIISVSLLAAEKLSDISPLFFTGVLEAVVAALFMNIYIVGLNQLSDVEIDKINKPYLPLA

SEQ ID NO: 81 - protein, 192 aa, Artificial Sequence
  misc_feature (1)..(192): Public GI no. 61808320_T
MDSLLLRSFPNINNASSLTTTGANFSRTKSFANIYHASSYLPNASWHNRKIQKEYNFLRFRWPSLNHHYKSIEGGCTCKKCNIKFVVKATSEKSFESEPQAFDPKSILDSVKNSLDAFYRFSRPHTVIGTALSIISVSLLAVDKISDISPLFFTGVLEAVVAALFMNIYIVGSNQLFDVEIYKINKPYLPLA

SEQ ID NO: 82 - protein, 182 aa, Artificial Sequence
  misc_feature (1)..(182): Public GI no. 51536170_T
MDSLRLRPSLLAARAPGAASLPPLRRDHFLPPLCSIHRNGKRPVSLSSQRTQGPSFDQCQKFFGWKSSHHRIPHRPTSSSADASGQPLQSSAEAHDSSSIWKPISSSLDAFYALSIVSVSLLAVENLSDVSPLFLTGLLEICFVTLVQAVVAALFMNIYIVGLNQLFDIEIDKVNKPTLPLA

SEQ ID NO: 83 - protein, 180 aa, Artificial Sequence
  misc_feature (1)..(180): Ceres CLONE ID no. 1789748_T
MDALRLRPSLLSARPGAARPRDHFLPPFCSIQRNGEPRVCFSAQRTQGPSLYHCQKSFDWKSTYSRISRQSTSTSINASGQPLQSEPEAHDSASIWRAISSSLDVFYRFSRPHTVIGTALSIVSVSLLAVQSLSDISPLFLTGLLEAVVAALFMNIYIVGLNQLFDIEIDKVNKPTLPLA

SEQ ID NO: 84 - protein, 180 aa, Artificial Sequence
  misc_feature (1)..(180): Ceres CLONE ID no. 395119_T
MDALRLRPSLLPVRPGAARPRDHFLPPCCSIQRNGEGRICFSSQRTQGPTLHHHQKFFEWKSSYCRISHRSLNTSVNASGQQLQSEPETHDSTTIWRAISSSLDAFYRFSRPHTVIGTALSIVSVSLLAVQSLSDISPLFLTGLLEAVVAALFMNIYIVGLNQLFDIEIDKVNKPTLPLA

SEQ ID NO: 85 - protein, 180 aa, Artificial Sequence
  misc_feature (1)..(180): Public GI no. 81295658_T
MDALRLRPSLLSVRPGAARPRDHFLPPCCSIQRNGEGRICFSSQRTQGPTLHHHQKFFEWKSSYCRISHRSLNTSVNASGQQLQSEPETHDSTTIWRAISSSLDAFYRFSRPHTVIGTALSIVSVSLLAVQSLSDISPLFLTGLLEAVVAALFMNIYIVGLNQLFDIEIDKVNKPTLPLA

SEQ ID NO: 86 - protein, 185 aa, Artificial Sequence
  misc_feature (1)..(185): Ceres ANNOT ID no. 1478147_T
MESLLPGSFPQLSSPLPSGGNYLGETKVKVGHSPREVQVYRCRARKILERQCVVRFQRHHSGFSVKKSTIYQENNAKFLVHAASGQPFESESGAYNPESTSKSVKNALDAFYRFSRPHTVIGTALSILSVSLLAIEKLSDISPLFFTGVLEAVAAALMMNIYIVGLNQLTDIEIDKVNKPYLPLA

SEQ ID NO: 87 - DNA, 1091 bp, Arabidopsis thaliana
  misc_feature (1)..(1091): Ceres GEMINI ID no. 350A6
aaaaaaagataaattacaaaatatcattttccttatcttattgacttgtcaagattctcttcttcttcttcttcttcctcctcctccaaactcagttccctccgtccatggcagcaaccttacctctatctccgatcaatcatcagttgtgtcggttcgggaacaactctttgacgactcaccggttctgttctcctggcttcttgatttcttctccttgtttcattggtttgaccggaatgggctctgctactcagttacgtgctcgtcgttctctgatctcttcagcagttgcgacgaattcgctgttgcatgacgtcggagccaccgtggcagtgcttggtggagcatacgcgcttgtcttaagcttcgagagtctcaccaagcgaaacgtcattcaacagagtttgagcagaaagcttgtgcatatactctcaggtctgcttttcgtacttgcgtggccaatcttcagcggatcgaccgaggctcgatactttgctgcttttgttccgttagtgaatggcttaaggcttgttattaacggactatccatttccccaaattcgatgctaatcaaatccgtcacaagagaagggagagcagaagagttgcttaaaggtcctttgttctacgttctagctcttcttttctctgcggttttcttctggagagagtctcctatcggtatgatctcgttagcaatgatgtgtggtggcgatggaatagctgatataatgggacgtaagtttgggtcaactaagataccttacaacccaagaaagagttgggcaggaagcatctccatgttcatcttcggcttcttcatctccatcgcattactttactactactcaagccttgggtaccttcacattaactgggaaacgaccttgcagagagtagcaatagtctcaatggtagccacggtggtcgagtcgctacccatcaccgatcaattagacgacaacgtttcggttcctctggctactattttaactgcttatttaagtttcggatattagattaatccttcataaacgaatgtatatatacgtattttttatgaatccgaccttacaaatgtttccacc

SEQ ID NO: 88 - protein, 304 aa, Arabidopsis thaliana
  misc_feature (1)..(304): Ceres GEMINI ID no. 350A6
MAATLPLSPINHQLCRFGNNSLTTHRFCSPGFLISSPCFIGLTGMGSATQLRARRSLISSAVATNSLLHDVGATVAVLGGAYALVLSFESLTKRNVIQQSLSRKLVHILSGLLFVLAWPIFSGSTEARYFAAFVPLVNGLRLVINGLSISPNSMLIKSVTREGRAEELLKGPLFYVLALLFSAVFFWRESPIGMISLAMMCGGDGIADIMGRKFGSTKIPYNPRKSWAGSISMFIFGFFISIALLYYYSSLGYLHINWETTLQRVAIVSMVATVVESLPITDQLDDNVSVPLATILTAYLSFGY

SEQ ID NO: 89 - protein, 304 aa, Arabidopsis thaliana
  misc_feature (1)..(304): Public GI no. 7406453
MAATLPLSPINHQLCRFGNNSLTTHRFCSPGFLISSPCFIGLTGMGSATQLRARRSLISSAVATNSLLHDVGATVAVLGGAYALVLSFESLTKRNVIQQSLSRKLVHILSGLLFVLAWPIFSGSTEARYFAAFVPLVNGLRLVINGLSISPNSMLIKSVTREGRAEELLKGPLFYVLALLFSAVFFWRESPIGMISLAMMCGGDGIADIMGRKFGSTKIPYNPRKSWAGSISMFIFGFFISIALLYYYSSLGYLHMNWETTLQRVAMVSMVATVVESLPITDQLDDNISVPLATILAAYLSFGY

SEQ ID NO: 90 - protein, 304 aa, Arabidopsis thaliana
  misc_feature (1)..(304): Public GI no. 28393229
MAATLPLSPINHQLCRFGNNSLTTHRFCSPGFLISSPCFIGLTGMGSATQLRARRSLISSAVATNSLLHDVGAAVAVLGGAYALVLSFESLTKRNVIQQSLSRKLVHILSGLLFVLAWPIFSGSTEARYFAAFVPLVNGLRLVINGLSISPNSMLIKSVTREGRAEELLKGPLFYVLALLFSAVFFWRESPIGMISLAMMCGGDGIADIMGRKFGSTKIPYNPRKSWAGSISMFIFGFFISIALLYYYSSLGYLHMNWETTLQRVAMVSMVATVVESLPITDQLDDNISVPLATILAAYLSFGY

SEQ ID NO: 91 - protein, 307 aa, Zea mays
  misc_feature (1)..(307): Ceres CLONE ID no. 1377623
MAAALPLSPVSHQLCRRSNRFWYNALTPRFCSPVFSTTSPCLIGVKGIGSSSQLRPRHPLISSAASTDYLLHDVGATVAVLGGAYALVLLFESLTKRDVIPQRLSRKLVHILSGLLFVLSWPIFSASTEARYFAAFVPLVNGLRLVVNGLSVSPNSTLIQSVTREGRPEELLKGPLFYVLALLVAAVFFWRDSPTGMISLAMMCGGDGIADIMGRKYGSYKIPYNPRKSLAGSISMFIFGFFISIGLLYYYSSLGYLHMNWETTFTRVAIVSLVATLVESLPITDQIDDNVSVPLASILAAYLSFGY

SEQ ID NO: 92 - DNA, 921 bp, Populus balsamifera subsp. trichocarpa
  misc_feature (1)..(921): Ceres ANNOT ID no. 1518536
atggccctctcgtcttgcactcttaccctctctcactcttctttacgccgccacgtacacaaatacaccaccactatccacctcgcccctcctccgtctcctcctcctcactggaacttgcccaatccaccagtacttcctttccgacgtctcgtttcctttcctccttacattccttgctcctccctcatttccgccaccgcacctctacttcaagacgccggtgccacggctactgtacttgctggtgcttacagtctcgttcgcacttttgatactctcactcagcgaaatctcattcaacagagtttgagcagaaaactagttcatatactttcgggcttgctttttgctgcttgctggcctattttcagcacctcaacacaggctcgctacttagcttctgtagtacctcttgtgaattgcttaaggcttattgtaaatggcttctctttggttactgatgaagggctcattaaatctgttactcgagaaggaaatccggagttgctgagaggacctttgtattatgttttgatattgattttgtgtgctcttgttttttggcgtgagtctccaactggggtgatctctttggccatgatgtgtggtggggatggtgtggctgatataatcggtagaagatttgggtcattgaagcttccttacaatcaacacaagagctgggctggtagcatatctatgttcatctgtggcttcttgatttctattgggatgctgttctactattcagccctcggatatttccagttggattggacatggacaattcagagagtggctttagtggctttagtggcaactgttgtagagtcccttccaattacagaggtagtagatgacaacataactgttcctttagtaagcatggtggtatcaatgctaagcttcggttactag

SEQ ID NO: 93 - protein, 306 aa, Populus balsamifera subsp. trichocarpa
  misc_feature (1)..(306): Ceres ANNOT ID no. 1518536
MALSSCTLTLSHSSLRRHVHKYTTTIHLAPPPSPPPHWNLPNPPVLPFRRLVSFPPYIPCSSLISATAPLLQDAGATATVLAGAYSLVRTFDTLTQRNLIQQSLSRKLVHILSGLLFAACWPIFSTSTQARYLASVVPLVNCLRLIVNGFSLVTDEGLIKSVTREGNPELLRGPLYYVLILILCALVFWRESPTGVISLAMMCGGDGVADIIGRRFGSLKLPYNQHKSWAGSISMFICGFLISIGMLFYYSALGYFQLDWTWTIQRVALVALVATVVESLPITEVVDDNITVPLVSMVVSMLSFGY

SEQ ID NO: 94 - protein, 302 aa, Glycine max
  misc_feature (1)..(302): Public GI no. 76443937
MTLLSSHLLVFSAVHHRAPPTTTTRNSPTTNHTVRFLCSPGVPPAVRLDQRLPRFVVPGAGAEDLLYNAGATVGVLGGGYALVRAFDELTRRNILQQGLSRKLVHILSGLLFLVSWPIFSNSPKARYFAAFVPLVNCLRLLVNGLSLASDEGLIKSVTREGDPLELLRGPLYYVLILILSALVFWRESPIGVISLAMMCAGDGIADIIGRRYGSMKIPYNEHKSLAGSMSMLVFGFLVSIGMLYYYSVLGHVQLDWASTLPRVAFISFVATLVESLPITKVVDDNISVPLATMAVAFFTFHH

SEQ ID NO: 95 - protein, 302 aa, Glycine max; identical to SEQ ID NO: 94
  misc_feature (1)..(302): Ceres CLONE ID no. 464672

SEQ ID NO: 96 - DNA, 1042 bp, Gossypium hirsutum
  misc_feature (1)..(1042): Ceres CLONE ID no. 1940214
agagagctcggcggtcttgttccacattctctcttctatttcatcagtttctccgctgctaccatgagcctctccttatccttcactcatccaatcttaagccgccacgtctattccgccgtatttcctcctccccgttttctcttcctctctcctctcatccccaccacgtcccgtttccctattctctaccgcgcgccccaacgcgccaccgcactctcagccaccgctgtaaccgcctctatcttccgagataccgctgcttccgcctctgtctttgctggcgcttatgctctcgtcttcaccttcgatattctcactcaaaaggagctcattcagcagaatttaagtagaaaattggtgcatatattatctggattactttttgccatttcctggccaattttcagcaacgccgatgaagctcgttactttgcatctctggttccacttttcaattgcttaaggcttgtaattcatggtctctctttgactgatgatcaaagcttgatcaaatctgttactcgagaaggaaatcccaaggaattgcttagggggcctttgtattatgttgcgatgttgatgttatgtgctcttgtgttttggcgtgaatcccccgtgggtgtcatctgcttggcaatgatgtgcggcggagatggtgttgcagacataattggaagaaaatatgggtcgtccaagattccttataatcaaagtaagagttgggttggcagcatttccatgtttgtttcaggattcatcatttctattgggatgctgtactactactcagctctgggttatttacaattggattggggatatacactgcatagggttgctttcatttctctagtggcaactgtggttgaatctcttccaatttccatgctaatagatgataatatttctgttcctcttgcttccatgcttgctgcctatttaacttttggtcactaatttcttcctggctaatctacttttatattcattttatttcttttacttttttagaaaataagattatcgtactcgagtttag

SEQ ID NO: 97 - protein, 298 aa, Gossypium hirsutum
  misc_feature (1)..(298): Ceres CLONE ID no. 1940214
MSLSLSFTHPILSRHVYSAVFPPPRFLFLSPLIPTTSRFPILYRAPQRATALSATAVTASIFRDTAASASVFAGAYALVFTFDILTQKELIQQNLSRKLVHILSGLLFAISWPIFSNADEARYFASLVPLFNCLRLVIHGLSLTDDQSLIKSVTREGNPKELLRGPLYYVAMLMLCALVFWRESPVGVICLAMMCGGDGVADIIGRKYGSSKIPYNQSKSWVGSISMFVSGFIISIGMLYYYSALGYLQLDWGYTLHRVAFISLVATVVESLPISMLIDDNISVPLASMLAAYLTFGH

SEQ ID NO: 98 - protein, 303 aa, Zea mays
  misc_feature (1)..(303): Public GI no. 76443931
MAAAAAWTGAASPNSLLLSRSPPHAAALAPSPGSSMRRRLLLGVGTPAVAALAAAAPPAVLQDGAVTVLITAGAYSLVRVFDELTERRLIEKSLSRKVVHVLSGVLFMSSWPLFSNSTEARYFAAVVPFLNSMRLLIYGLRLYTDEALVKSVTREGKPEELLRGPLYYVLVLLFSVLVFWRESPIGIVSLSMMSGGDGFADIVGRRYGSAKLPFNRKKSWAGSISMFISGFLLSAMMMLYFSSLGYIDVIWEEALGKLALVALAATVVECVPVTEVVDDNISVPLATMLVAFLLFSSNRTIVN

SEQ ID NO: 99 - protein, 303 aa, Zea mays; identical to SEQ ID NO: 98
  misc_feature (1)..(303): Ceres CLONE ID no. 287069

SEQ ID NO: 100 - DNA, 1032 bp, panicum virgatum
  misc_feature (1)..(1032): Ceres CLONE ID no. 1780314
aacgggctcgccgcggggccacaaaggacacgagacgagctgcacaaatcgctcgcttgctaggggcgcccaaacccgggcgctcctgcaaacctgtcgctaccgagccacatggctgcggcgggcgccgcctgcgcctcaccccacagctccctgctgctctcgcgctcccgtccccacgggctttcgccggcgagctacatgccgcggcgactcatcctcggcgtcggcacctcggccgtcgcggcgctggcggtggccacggcgccgccggcggtgctgcaggacggggcggccacgctgttcgccaccgccggcgcctacgccctcgtgcgcaccttcgacgtgctcaccgagcggcggctcgtcgagaagagtttgagcaggaaaattgtgcacgtcctatccggcgttctgttcatggcatcctggccactcttcagtaattcgacagaggcacgatatttcgctgcggttgttccgttcttgaactccttgaggcttctgatttacggactacgcctttacacagatgaggctcttgtaaaatcagtgtcacgtgaaggaaaaccagaggaattgctgagaggtcccctctattatgtcttggtgctactcttcagtgttttagtcttctggcgcgagtccccaatcggggttgtttccttgtcgatgatgagcggtggtgatggttttgctgacattgtcgggagaaggtatggctcagtgaagctgccattcaatcagaagaagagctggatcgggagcatctcgatgttcatttctggtttcctgctatcctcgataatgctgttctacttctccagccttggttacattcatgttagctgggaggaggcatttagtaagctggctcttgttgcgttggcagcaaccgtggtggagtgtattcctgcaactgatgttgttgatgacaatatatctgttcctttggcctccatgttggtagctttactcttgtttggctccaacacacaatgattaatccataataataagagtacattattc

SEQ ID NO: 101 - protein, 296 aa, Panicum virgatum
  misc_feature (1)..(296): Ceres CLONE ID no. 1780314
MAAAGAACASPHSSLLLSRSRPHGLSPASYMPRRLILGVGTSAVAALAVATAPPAVLQDGAATLFATAGAYALVRTFDVLTERRLVEKSLSRKIVHVLSGVLFMASWPLFSNSTEARYFAAVVPFLNSLRLLIYGLRLYTDEALVKSVSREGKPEELLRGPLYYVLVLLFSVLVFWRESPIGVVSLSMMSGGDGFADIVGRRYGSVKLPFNQKKSWIGSISMFISGFLLSSIMLFYFSSLGYIHVSWEEAFSKLALVALAATVVECIPATDVVDDNISVPLASMLVALLLFGSNTQ

SEQ ID NO: 102 - protein, 268 aa, Zea mays
  misc_feature (1)..(268): Public GI no. 76443929
MRRRLLLGVGTPAVAALAAAAPPAVLQDGAVTVLITAGAYSLVRVFDELTERRLIEKSLSRKVVHVLSGVLFMSSWPLVSNSTEARYFAAVVPFLNSMRLLIYGLRLYTDEALVKSVTREGKPEELLRGPLYYVLVLLFSVLVFWRESPIGIVSLSMMSGGDGFADIVGRRYGSAKLPFNRKKSWAGSISMFISGFLLSAMMMLYFSSLGYIDVIWEEALGKLALVALAATVVECVPVTEVVDDNISVPLATMLVAFLLFSSNRTIVN

SEQ ID NO: 103 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 104 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 105 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 106 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 107 - DNA, 1000 bp, Arabidopsis thaliana
  misc_feature (1)..(1000): Ceres Promoter PT0613
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 108 - DNA, 351 bp, Arabidopsis thaliana
  misc_feature (1)..(351): Ceres Promoter PT0625
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 109 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 110 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 111 - DNA, 998 bp, Arabidopsis thaliana
  misc_feature (1)..(998): Ceres Promoter PT0660
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 112 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 113 - DNA, 999 bp, Arabidopsis thaliana
  misc_feature (1)..(999): Ceres Promoter PT0672
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 114 - DNA, 1000 bp, Arabidopsis thaliana
  misc_feature (1)..(1000): Ceres Promoter PT0676
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 115 - DNA, 998 bp, Arabidopsis thaliana
  misc_feature (1)..(998): Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 116 - DNA, 1000 bp, Arabidopsis thaliana
  misc_feature (1)..(1000): Ceres Promoter PT0683
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 117 - DNA, 1000 bp, Arabidopsis thaliana
  misc_feature (1)..(1000): Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 118 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 119 - DNA, 1000 bp, Arabidopsis thaliana
  misc_feature (1)..(1000): Ceres Promoter PT0708
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 120 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 121 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 122 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 123 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 124 - DNA, 1000 bp, Arabidopsis thaliana
  misc_feature (1)..(1000): Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 125 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 126 - DNA, 763 bp, Arabidopsis thaliana
  misc_feature (1)..(763): Ceres Promoter PT0837
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 127 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 128 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 129 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 130 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 131 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 132 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 133 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 134 - DNA, 999 bp, Arabidopsis thaliana
  misc_feature (1)..(999): Ceres Promoter YP0019
gatataagtagaatcattttttgccgccgtttctcgctaacacaccgaaaactgaatcaaatctcctagctcttctacgcaaaatcgagtgcatcgacaatggcggaacgtggtgtcgaacgtggtggagatcgcggcgatttcggacgtggattcggtggtcgcggcggtggaagaggtggtccgagaggtcgtggtcgccgtgcaggtcgtgctccagaggaggagaaatgggtgccagtgactaagcttggtcgtctcgtaaaggaaggtaagatcacaaagattgagcagatctacctccattctctcccagtcaaggagtaccagatcatagatttactcgtcggtccttcattgaaagacgaagtgatgaaaatcatgccggttcaaaaacaaaccagagccggtcagagaacgagattcaaggccttcatcgtcgtcggagatagtaacggtcacgtcggattaggagtcaaatgctccaaggaagttgcgacggcgatcagaggcgcgatcattctcgcgaaattgtctgtggttccgatacgaagaggttattggggtaacaagattggaaaaccacatacggttccgtgtaaggtaaccgggaaatgtggatctgttactgtacgtatggttccagctccgagaggttctggtattgtggcggctagagttcctaagaaggttcttcaattcgctggaattgatgatgtctttacttcttctagaggatccaccaaaactcttggaaacttcgtcaaggtatgtactttcacaatggctgttttggtttgatgaactctgaattaggcagtgaaaaagtaatcattaccagttaagtgaatttatattgaagattaggatttagctgattgtattggtttgagcatgtgagtttgtgtttaagattgcttgaattgaaatgctttaggttgtttgattacgctaaattctgactaatgtaattcaaattgttgttgtttttttttggtc

SEQ ID NO: 135 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 136 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 137 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 138 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 139 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 140 - DNA, 1024 bp, Arabidopsis thaliana
  misc_feature (1)..(1024): Ceres Promoter YP0092
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaat

SEQ ID NO: 141 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 142 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 143 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 144 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 145 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 146 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 147 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 148 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 149 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 150 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 151 - DNA, 1000 bp, Arabidopsis thaliana
  misc_feature (1)..(1000): Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 152 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 153 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 154 - DNA, 1004 bp, Arabidopsis thaliana
  misc_feature (1)..(1004): Ceres Promoter YP0128
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 155 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 156 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 157 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 158 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 159 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 160 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 161 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 162 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 163 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 164 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 165 - DNA, 999 bp, Arabidopsis thaliana
  misc_feature (1)..(999): Ceres Promoter YP0275
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 166 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 167 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 168 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 169 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 170 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 171 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 172 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 173 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 174 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 175 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 176 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 177 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 178 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 179 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 180 - DNA, 667 bp, Arabidopsis thaliana
  misc_feature (1)..(667): Ceres Promoter PD1367
acagttttcttttctcatcttacaacaagtttccaggaggatagagacataaacgaagctcggattgtatcgttctttttagcttttattcacatccgaaagtcctgtagtttagattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttctcctctttcacttcatcacttggcttgtgaattcattaattcctgcgaa

SEQ ID NO: 181 - DNA, 1836 bp, Oryza sativa
  misc_feature (1)..(1836): Ceres Promoter p530c10
gcctctcgaccacgagtttagcacttgtgcaacatatatgcgtgcgatgaacatctactgatgcgccatgcgaattttagcgttcgttcatgacgcttccaacggcacagaggctgagcagcagcatgcatgcatggctcttgtgaaaacaaaaaaggttactggtaaatgacatgctgctgtagctagctagcagaatgcaaggcccatgcatatgcaatgctatgcgacaagtacagtaccagcatgtatggtagccagctaactaatctatcagcagaggcagcaagctcgtgcatggtgtgatgcacttctctccagtaatctagtggtaattttcacccaaagcgttgctcatatggacagtaattagtaatattaccaaggttcacaatcccgttacctgaccaaatactactcacgaatggtatctctggttttcgttaaaaccgttggtaaaccagcaaaaatagacaaaatttgtcaaaattttaaattttagttttttttttttaacttagccgggaaaccttgaagtttgtgctgtcgagctgtcctgggaaggacggttttggttgggattgtgaaccctggttactgcacttcatttttgaacagatattagtgcaacagacaaatgccaacgcatttttttctgtttaccggcaagctgaagcttttacgatccccatacagccgttgctgcaaacctgccaagaaagagcagcagaaacaggtgtcattttgtggtggaaagccaagtaaagtaaacagaagatggaagatagtgaggaccagggagtgaggcaggggacacatggcccacgcctccctgcacattttcgtgtataaatacaggtggatgcatcgctctcccagcatccatcggttctctgctctgttcatccatagagtttcctcctcttctcctttagtgcaaggtagagaagagcatgtgtgtgtgtgtgtgtgtgtgaactgtgaagtgcagagtgcttctgtagttctgtgttatgtccatagtgatcttgttaggattgttgctatggatgcatgatgttatggttgatctctgaattacagtagggacttttctgagatctctggattagtggggggtgctaaatttttttctggttgcatcagcttgggtttctggtattggtgtgggttcttgctctgaattttggttcagaatgtcgatttgtttgtgtttgttctctgaagttgagagtagctatgatccatccagcacagaactgcaggtcctgcctgccggctgcatatacaggacatgccattttgcaagctctgggcttatggtttctcttttggagttcttcttcttgcatgatctgtgttctctaacaaaggaagcaagatttagcaactttattcagagacaagaaaaggatctggcaaccttttgtttctgttttatcctactcgtaaagattgttatttaagcaaaaatttcccaaaagttttaaatataatttccatgatgtgccactctcatgtccttgaacctggcactcattatgggctcctcagaagtgctgtagctaatgtcactaatcttttgtatctttgttcatagtcttgtattttatgatgcttatccctttgtgctttccatgtttgatgtccaaatgtcatggcaatgtttttgacttctagtaggggttttagtacctttttgttagataagtacatccaaattctgtttatttattcaaaaatcattctgtttattcactgaaaacatttgtccattcaatggactcataaactgtctgtgtttttcaggcttgaggatccatctagaagatagca

SEQ ID NO: 182 - DNA, 3000 bp, Oryza sativa
  misc_feature (1)..(3000): Ceres Promoter pOsFIE2-2
gcttaacacatgaactaccaaaatatactgatcactttgttctagtcatacataccttaagtcattttattctgcagtgtttggattggagggagcattctagcatcccttgggtcgttccagcaaatgtggttctccaaagcagagtaagcacaacacagtattttaggttatgtttcccctatctcgtcacggacagctcacaagttaatgtgatttatctcactatagatacgaagaacatggagtatcctacatccaaaggaagtgcccatgaagttgtggagcatcgctacgatttgtgaccaaatttgggtgcatgtgggcaatcgtattacagccaccctgttgttgatctatatcgactattatccgacgatatttatcattatattatgactagttagtttgtagattttgagagggcaacataagaagcaatccagcttaacctgttatgttcttgatggtagattctagttcatgtgttgaatctgttctccctgctgtagaatgtatcgagttgctgctctctactctgtacttttagaataccttttcaatcatttggagtcagctgattgttgtactacttatacgccacctgattagtcatgtcaacaattaaacttgagcactggttaagttaagagtggcctgattgtagttgataatcacattttattcgtagacattgtatgctggatctttatcagccaccgtcagatcatcctctgtaataaatcttcatcagacgtgtgtgccaatcgcaaggaacacgaaatgcatccgaaatgttactctgagttaatcaatactataattcttggtcaaattaattatttatatctataaagtttaaattaaatttaggaaaatgaattcatgcaaatcttgtggtaagttgtcaatttcataaaaaatccagcttactactccctttttaggagtgtgttgtggctgcacacttctgccttttgatatatacggttctattctcggtgtactcctttattattattaaaacaatcccagttacttggtaagtgctaatcacgaatcaaagtcaacataacaaatcatgtgcgtacagctataactcgattacacaaacaacaaaattcatatttgaacataaatccagttgtagcatatctggtagtataaagttttttttttgtatagaagagttttaatttctgtaagttttggaaagcatttaatcctagaaattgtagtgtagctcaactaaaaaataaatgaacttgaatcgaaattgggttgtatcataaatctttaccactcaaacgaatatttatcctaaaccacaaatgactcttttcatcaaggaatgttttgttttcagcattttaaaaaaaaacttttctaatatggttttcatgtttcgttcttttgaaatttaacatctatttaatttgcacggctccataaattcaacggatacatattctgaataattactaaggaggcatatatcggctctcttaatacaaccgcttgtttctcaaaatttattttgagttttgtctacacattctcaaggacggtacaaacacactatagatgttcacaattttttttttctaaagttgattgatggacaaatgtttgaacatataaacatataagcactgaatatttgcttatgcaggaggtatttatatcaagttcgatactttactaccatagtccctaggacactaaaatgccttcaatgatctgatgaagcctaagagagaatattgatcagtggagcgacttgcaactacacatggcacaagtagactagacacggtatatattcatattaacttgttaaaattttactacttaacagttcacttgtggtgcatccatatcaattcttacttacacaatatttgtaaaaacaacctaacactataggatgacctagacaacctttatgtcaatcacacttagaagatgatcgtctttttaataaataatgtgtactacacaccatgctctccatatagatcaagatctacaaacccttccacttataaaccttaccaccaaaaactcattaagttgcttcatttatctatgctattaagaaaaaaacttatttcgtttatgccatttctagaaatggctagtcacactattcacaatattatataataaataaaagtttcaaatattcatccaccaaaaatcatcaagtcgtgggacttatatgttaattagagaagtccctttgggtgcaatcgattttggaaaccctaaattttttctatacatagaagagagagatgtctagttgcaattgcttttgcgatgtgccaaccacccttctagctttcatccacgtctacttaattgccattcttcttcttctttttcttcactattactacctcctatcttagcgaatcttcttcttcttcactattactacctcccaccttagtgaattcatcctcattgttcacaatgacattgctaagttaactaggtatgctaagtacacaattagaatataacctagagcctttgtttccatcatacttaaaagatgacatttttatatagataaagtgtgctactcacaaggcttactatatatatgtatgatacacacaaactccacaacccaaaactctttcaagttgtgtggcccatctatgctattaaaaagcccatttagcccatccaacatgagaaaccctagggttttttccctataaaagatacctaggttattgttgcttttccaccccgcccgccgccgctccctattcctatttaatcccatctctcttcctcatcaccgctctcctctctccaggcaagaggtacgcactttttgtttcggatttgaaatctttgcttcgttttactatcattggtcataagttcttttttgaagatgtttgagaataagtttatcattgagattatcgtcacttgtgataggaagtacgcaacctcaagccggacaagacgtgagcaaag

SEQ ID NO: 183 - DNA, 2023 bp, Oryza sativa
  misc_feature (1)..(2023): Ceres Promoter pOsMEA
gagagcagaacatagtagccgctgttttctgggggtgcaatttgtgcaagatcgctatccttatggaccatgcaagcaccaagcaatattaagccaggtccaacagcggtcttggggaattcagaaatgagcttaaaaacctccttgagctggccagctcagccaaggaggtccatcatgcatgtgcatgctcaatacttggaattattgcaaaatgatcggtcattgactggaagactttgcgcccttcctcagccaaccttatgtggctgcatgcatagagtaccaacaggaaggtagcgtttgttggaataaggtttgcatccagcatgtccttgtagagcttcaaagcctcagcaccttggcccatgaaggccatatccagctaattgcattccatgagaccacattcttgctatccatactgttgaagtgaagatgctccgagcttcggaaatgcttccacactatgcatacatgtcaatgagcactgtcatgacataaacattgggccccaagtcctcctcagcgataatcctatgcagccactttcccagggacaaagctccaagctgtgcacacgctgaaagagagctagaaatgatgattggatttggtcacacgctaagtaccagcatttgctcaaagagggcaattgccatctccgtccagccattctaggcataccctggtattattgctttccatgattccgattccgtggtcttctatggcatcgcattgaaggccttccttgcagactccatatcatttaacctacagtacaatatggtaattgctgtcgacactggagaattcgcagtaaatccagacttgagaggaccatgtaagcattgatcaagcagttcattcccaaacagactatacgggatcagtgccagtgctcgagtttggcttcaattccaaggccatcaacccaataaacagattaactgatgaaccaaccatgcaattcgccgagcaaacatagattaagcattgtaggcaaccaaatctggattctccatcaagtcaaagagacgccatgcagaattccacatccccgctgtatacaccgagatcaaccggtcagaacatgctcatactccgccaaccctctcttcagaacatgctcatactccgccaaccctctcttctctgcaagaggcatcctccccaattccccattgttatatctgttgctggtaagaccgttgccagcgtggttgtgtcagaccgaacagactctgcactcgccatcctcacgaacgactccagggcctccgaaccaggaagcccggccggccatcagcgtgttccacataacggtatccggcgactgcacagtgtcgaacaccttgcgtgcgtggtcacctctggacagcatgaagcgtacaggctacagcttggccaatgcggacgccacgaacgtgtcggcggcgtaacccgcgcgtgcagcgcgccgcgcgcgggctgcggagtcggttggagacgacacgccgccgccatgagagcaatgagcgaggtggcggcgaaggcgaaggagaagtagtcgaggcaagcggaagagaaggcggcagcggagaaagcgatcggggcggcggaggaggtgggtgggagggagggacgcgtagcggaggtcggaggaggagggagctgaggtttccggggcgggggtcgagagggtagtgtacggaggcgagggacacggcgaggatctggtcgaggtagcgcagtgtgaaggaaagcgcgatgaggcggagggcgccggcgaagagcggcgcggcggatagcgggaggaggcggcgccggcggggtctcatccgattggaaacagattgggaagggggagggggtaggaatacgtggcgtcggcagtattaggtagagagagaaaccctttccatcctttgtctcttagccccgaaggagagagaaaaatcagaaaaaaaaaaccctccgcgtgtgggggaagcagagctccggacgctggcgccgctcgcgccaccgcacccgcaccgcc

SEQ ID NO: 184 - DNA, 2034 bp, Oryza sativa
  misc_feature (1)..(2034): Ceres Promoter pOsYp102
gaacgacccaaacgcgtaaatggtggtactggtttccctgctttgccgagtaccagcagccacgaagaacgttacacaatcgagtacaaaatctataagagcaagtttaatagcatagccaaatactacctctaaatcatctatagccaatttaatagttcatttattcaataattacttataaacatatactacaatcattaatatatggtcttacttcttatacacataatattttggagtccgtgttacagctggctataaatataagggattttggttggatgtggtacatcctattataatgaatctagacatgaaacctgtccaaattcatcgtgctaggatacgccacatctaaccaaaatctcttatctttagggatggagagagtaataattaaatgaagctaggtagagtttcccggtcaatacgcttgcgtgtgcttataagagcatggccaacagtttcccgatactcttcccaatatcagttttgaggagttttgttggaaaaaatcgctccaacagtagacctaaatcacccctaaaagcttggcgtttccaaacccgcatatttcgttctccacttgtagggaagagactcggcgcccaatccttcaaccgcatgcacttcgcgcgcgctgtgtgaaaattttcctaccaggttcttctttgtgcgttcgtctacctgtgagtcaatccatcacgccagcagcctcatcttccccgcagctgtctgggaaagcagccatggctcccccaagcttccccagcgtcgacatttttttctcagcggcagcgccagacccatctccaacccaattgggcggaccttcgtcggcgctcccccagcaccaccaccgactcgaatcggccgtcgcccctattcatctccaatcgtccctcgaccctaccgcatcctgcagcacagcctgtctctcgcgtcagactggcgctgcgctccccccggtaatgtgcaggcgacaaaggccccatgcgatgcgaccagcagccggcgacaaccggaggtgcccagtcgctggccttcatcgaatcatcgtgcacctcggtcggagtcgatttctgattgttgctgctgctcaaatctggagcttgctattgctgagaactgcttggtggtggtactggaaatttgttgtttgctggctgatgaaaactgttgttctttgctgctaaaaactgctgcttgctagtactgaaaagtactattgcagctgctgaaatatcttgctgcttgctgctgaaaacttcaagttgttaacaccgttcacactaaaaaagctgaaattttttttctgggctgaaaaccccattgttgatgattgcagaaccaatatttttccatgtaaaatacaggagatcgtggtaataatcaagtgaaatatcattttggggcaaatactcagatcgtacctgaagccaatggaaacattgttcaatgcttaaactgtcagttatgatgtcaaagagattgatcactgaatgtcctgaaaggagccgtgaggaggatgcagcattgcagcgtgcgcgagcgtgagtggaggagaggaatgacgattctgttggtagttgtcgatgtggcctactttttttgttttgaggattaaattttgggaatctcttggagataaaaggtattctcataccttaaatcctttttagagatctaaaaaaaatgatttaggggattgaattttgggtggctgttggtgatgctctaagttgcacatcctggggaaaaacctccctaatccatcagcaaaccgatcaaccacccacgacaagtcgacgccaccgttttttttttctccctcctaagtcctaaccccacaaaaatcccgcgaactttcgtctcaccacgcgccgcgtgccccctacaaataccaaacaacacccaccacgtccactcacaaaccacgcaggaaacctcagaaaatcaccgtacgcgacgcgggcccaagaaaaccccgacagaaaccgcgcagcagcaacaccaccaccggcgtcggag

SEQ ID NO: 185 - DNA, 1877 bp, Oryza sativa
  misc_feature (1)..(1877): Ceres Promoter pOsYp285
ggcccgagttaaacgatcttccacgtgtcagcgaatcctagtcgttcgatgaatctgaatctgacttgtggtggttggacggccacgtgttaaaaaagggaaacgtccgcatcacccgatgctgggacatttgcaatttcgatccagctgtagattgaccagttgttactctcttttttttaacaccatacaaacgtaatactccctctgtcccaaaatataagtattttttttaacctcggttcagtcttcgaggtgctactttgaccaataatatttataaaaataagatgttttaaataaagagagttgcatattatgatagctcgtttaatgataaacaaagtaccatcaaatttacatgattaatctttttaatttatttgctattaatagttaaaatttaaaaagtttgacttcacactgttctaaaaatacttatattttgggacggagggagtacacattagagcaggtacaatagcagactagtagccagctataaacatattttaatgagataaaagatgagagagaacagcgggctacagatctgtagccagctgcagcacggactccaagacattgtgtgtgtatgacaggtgggaccatatattaatagtacagtaagtaactattgtatgaattggctattagattagctataggtgaattgtagctagtagtgggctatactattgaacttactcttatatctctcaatatctccagaaaactaggacgatatatattgatattaacaaagtcatcatagatatctcgctatcgacatatatattacctatcactgaaaaaataattaatcataaatgcaagcacatatactacgttcaacactgaatgtaggtagattggtagacgggttccaccgcaagaaaagcattgcaccagtgaagaaagaaacatcggaatttgtatgtagtttgttgtttgatgaattcttttgattaaaaaaaactaaaatcagagttgattcagttaatggtgttgcctacgatatacttccatatcatgatatcactgtagactatgaatcatatctttaattaaaactaaatcaagaaattaagtatgagacctcaactcaatgaagaatttctagttgaaaaacattcctagtgtgcgttcggatggaggtagggatcttctctccgttcatataaaaccggatggttcattagaacatgattaattaagcaacagttaatctaaaaataaattaatattttttaagaaatttttgtatagagatcttttgaaaaaaatacattggttagaaagcatactaataaaaagagaaaaataagaacatagtactatagtagaaaatgagaacttggagtatttgagaggatgggaaataagaagattaagaagatgcgtaaagtgaacggttaacgcatgattgattaattaaatattaattattttaaatttggaaaataaattagtatgatttttaagcaacatatatatatatatatatatatatatagaaaaacatagttttagaaaatataagcgtgtaaaacgatatgcaggaacgaaacgttgagcattcaaaatttcaaattgaacatatgaatcaagagagaataaaaaaagaggccttctaggctggcatggacaattggacatgttttcaactagggtttcaagcttcgagcatccacttttgtccttgcaaactttatacggcaaggcccgtgaatctagccccccacaccaccccacccgcccgcgccgcgcggccgcctcgcctcccctcccttctcctcctctccgcccccgccgccaggccgtccacctccgccgtctcctcccccattcgcacccaaggcgctggcgcggaaggc

SEQ ID NO: 186 - DNA, 1000 bp, Arabidopsis thaliana
  misc_feature (1)..(1000): Ceres Promoter PT0565
caccaaatatagtgttatttcaatactaaaatggtgttatggttggagatgccctaaagataaacatgacgagacacgagatttattaatttcttgatcaaccataacttaataacttaatattaatttcacttaataatttccaattaagtgaatctttacttcaccaaaagttcctaacgaactcttattttctagcatcaatattaccatgaactagcatcaatactatcatgaaaaattcctacttcctatccaactcttaataacaatgctagtcttaacaatattcatcaaaaacttgatatagaccttctaacttagccacgactagtatcggtgaataccaaaattaatgtattcatgagaacttgagatttctctaatgtattcttgttactaaacaagtaacaacactcaagaaatatcatgatcaaatattttactcataaactccatatttcacattttgaaaattttaaacagcaaatcacattgaattttcgtggtaaaagtatttaaaattgaaaaatagcagctcctgatttcaatgtataaatttatctttatatggtttatgtctccaacttattttaaaaaagagagaaagagcacccaaaaggtgaccgtttgaaattcgaatttatttccgtttgaaattcgaattcaaaaaaagtaaaccgaaccgagtctcgttactgactgtcacacattgtttccctaaaagctaattaacccatacgtggcgtaatataacaggtcagtgatcaatactaaataacagacatacacctttaaaattcgtgcacgctccaaaacaaaatctacacttcaaaatcaacggtcacgatcattcctcaaatttcaaaaaattatttaacctcacttccttcgctttgtttttaaaacctctctctctttctctttctctttcgccattaaaactctgtttcctttttcagagattctcagagaagattcattttaccctaagaaaaaa

SEQ ID NO: 187 - DNA, 999 bp, Arabidopsis thaliana
  misc_feature (1)..(999): Ceres Promoter YP0015
ttgagccttattgttgttattgacttttagccaatagaaagagatggaaattcaataattatccacaaaattccaaatcattggtgtacaaaaagatctaaggctgttatattttcaaaaaagaaagaaaagaaatgcaacaaatatggattaaactgtggtttgtaaattgagctttgcatgaaaactttatcactatgatttcactactccatatttattgactaaagtggcactaatgaatttcttaatcatgaaatcttgtatcaaaaagtactaaaataaacatgacattggcaattaggaaaattctaaattagaaattagtaaaaatgaaaggtgaaagggaaagatgatgatatgaattggttggtgaccaggagaaatgtatcccgatttttgcagacactttcagtgtccccattcatataattatggcccacctcgttaagatttttcattcaccaccataacaagatctaagcttagatttcatgtaattaaacatataatatacttgccaatactatctaataaagtatacttaagcaaaaattattactctagtgtaaggcgatgaaatataagtttagttgaaaatttatgtcgatataacaaagtataatgaattaagaccttggttttcgattaacaaactaattaaacactagttttgcctaataaaaccgggaatcgtattcaaaaccgaacgacaaaacaagggacaagttgagagacaaaaccaaatcagcatctttcttccagaaatgtcatgaccacatgacgtcatcttgacccttcttcattgtgatatctgtggataaagcgcacgtgtttaattcacgaaccttcgtagtaacgaaaaatccacaactttcatattttttaattacccactaaactaaaacaaatttggaaaaacatgaaaaactttttctttttttccaggttcgtgaacctcgtaccctctatataaacctcttaaccaccttccacata

SEQ ID NO: 188 - DNA, 999 bp, Arabidopsis thaliana
  misc_feature (1)..(999): Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 189 - DNA, 1000 bp, Arabidopsis thaliana
  misc_feature (1)..(1000): Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 190 - DNA, 999 bp, Arabidopsis thaliana
  misc_feature (1)..(999): Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaaacttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 191 - DNA, 999 bp, Arabidopsis thaliana
  misc_feature (1)..(999): Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataggcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaacttatatgagtaatagaaacgatcctaatattaggaattttagagattttctctcatctgtttcttaacttttcaatatttttattttttaaaattgtatgagtttctactaagaaactactgctggagttggtcttagcttcccaatgcttctccacctatatatatgcatatctccttcttaaaac

SEQ ID NO: 192 - DNA, 999 bp, Arabidopsis thaliana
  misc_feature (1)..(999): Ceres Promoter YP0080
aagcggcaatttagtaagaagtactcaaagtatcatttaccaaaagtatatggttttgggaagagttgttagggatgtattctttctaaacagatgatatgacgatgttcttgaaaactaatgttaaagacggaatctctggcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaattacgaaagaaaacaggaaatcaaatcaaaagataaagagaaggtaaaaaaaggcaagaagcactaatgtttaatatttatagttttctccattaaagaaaaagcgatgatgtgtgttctcatcttttgtgaaagtatatatattgcttttgcttttctcaaaagcaaaagactcatccaacaagaacaaaaaaaaaaactaaagctcaatccaaaagacgaagaatgcattggatactacaacttctttttcacttttctttcaaatttacaattatgattttcacaatacagtttattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgtttttatttttctgatgttaca

SEQ ID NO: 193 - DNA, 3000 bp, Arabidopsis thaliana
  misc_feature (1)..(3000): Ceres Promoter PR0924
atctataacgagttaacatgttgccagtttgaatcaagaagcttggatgatgaatgaatggatcggtttgtggtacaattcttaaaattgtagtagaggagacagagaaaaaacatgataagactttggtatttacaacttgacggagacaagacagtaagccaaatctgtcacaaaaacactcaaactcttttctcagtgttttgagtttaaagagagacttattcacttcccctttcgtaacacttatttgtctcccaaccaaacagtttctgtcctttcccttgtcctcccacgtgcatctttatatctcatgacttttcgtttctagatcttgaataatgtcttagtggattaggtttgttgtcggtaaattaggtgaccgtttttttcttatatttggaagatcgcgggatgaagcagatactgagtttcagggcatacacacctaatttgaaaatcattgttagtccaatttcactttaatcttgtttacaaaaaaattgatctgaaaatgttgatgggataagtaaaaatgtaagttttgctagtagtcatgatataataatagcaaaaccagatcaattttgagcaaaaggaagaaacaaaaaacagatcgatcccacgagcaagactaagtgtaaagtggttcccacaagagccatatggatatggtccttcaacttttaaagcccattacttcagtggtcgacccgacattacgccacgagtagtcacgcacgcacgactccgttcacgtgacattcacgttgatatttccccctctactctcttctgcttggttgatctaaaaaacatgaagagaccaacctaatttcatattaatatatgatatagacttcatactcaacagtcactttcgtaatccaaatccatatcttacgaaattagttcttaataaaggttgtggattaagttataatattgtgttaagagttaagacacagcatataaccttgtaccaacagtgctttattcttaaatggaaacaaaacatatgtcaatgtcaagcatacagctaaaatatcattatctaatattaagagtaaaacaagataattaaaaattgaaacaacaccatatttttatagctttacttatcgtatttttctagtcttcatggtaattgtgttgctttattttgtttataaatgaatttggttcgaccagatagtctaatatcagtttttaaacactggttttaataaaatcatatgtcggcaattcaacctgttacgttgtatgattgtatcctagtcaaataggggaggaggtactagtcgtttcaattagtttacgtaatcaatccaaagaaactataagctataaagatcctcaatttgttggttacaataaaaacaacagttgtcaaaatttatgtttataaaaagtaataactatgttccttcccatatagagcaaagtacctcaggataggcaaaccgtacttaatagcccttattcataatttgatccaactcttccccacaaaattgcaactgatgaagtcaatacttgtatagtgagtcaagctataaatgtctagtgatagttttgtctcttaaaaggttaacaaaagttatgacaagctgaaaaatcagagtttgctaggagtattacttacagttatcagtttaagtatcacatttatagtattgtatacaatgattcttaaattccaccttttccgtgcgaaaccaaattttctattggaaacatagaatgtaaacaaaaatatgggacgttgtccgttccaacattaaccaaacttgtctattactaatattcgtgttggtttgatgttggatgtctaaattcgttgaatcatgtgtctcttgacgaaatatgcatcttcttatttcttagtatagatgcactttatcattcttttagtacatgcttaatttttttttttaaaatatgttgattgtcatattgccaaaagtatgaattaaagacgcacatctaacacaagttagcagccgtaaatccttccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 194 - DNA, 1000 bp, Arabidopsis thaliana
  misc_feature (1)..(1000): Ceres Promoter YP0388
agaagtattcacgcaccaaggttatatttgtagtgacatattctacaattatcacatttttctcttatgtttcgtagtcgcagatggtcaattttttctataataatttgtccttgaacacaccaaactttagaaacgatgatatataccgtattgtcacgctcacaatgaaacaaacgcgatgaatcgtcatcaccagctaaaagcctaaaacaccatcttagttttcactcagataaaaagattatttgtttccaacctttctattgaattgattagcagtgatgacgtaattagtgatagtttatagtaaaacaaatggaagtggtaataaatttacacaacaaaatatggtaagaatctataaaataagaggttaagagatctcatgttatattaaatgattgaaagaaaaacaaactattggttgatttccatatgtaatagtaagttgtgatgaaagtgatgacgtaattagttgtatttatagtaaaacaaattaaaatggtaaggtaaatttccacaacaaaacttggtaaaaatcttaaaaaaaaaaaaagaggtttagagatcgcatgcgtgtcatcaaaggttctttttcactttaggtctgagtagtgttagactttgattggtgcacgtaagtgtttcgtatcgcgatttaggagaagtacgttttacacgtggacacaatcaacggtcaagatttcgtcgtccagatagaggagcgatacgtcacgccattcaacaatctcctcttcttcattccttcattttgattttgagttttgatctgcccgttcaaaagtctcggtcatctgcccgtaaatataaagatgattatatttatttatatcttctggtgaaagaagctaatataaagcttccatggctaatcttgtttaagcttctcttcttcttctctctcctgtgtctcgttcactagttttttttcgggggagagtgatggagtgtgtttgttgaatagttttgacgatca

SEQ ID NO: 195 - DNA, 283 bp, Arabidopsis thaliana
  misc_feature (1)..(283): Ceres Promoter PD0901
caaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagctttttaaaatatttctccacgtgcctctttgtgtttgtctatata

SEQ ID NO: 196 - DNA, 1000 bp, Arabidopsis thaliana
  misc_feature (1)..(1000): Ceres Promoter PT0623
aaagttattgacattttgaaaggaccgtaaatattaccaaaaaactgacggagttaggatcggccacgtagaaagggacaaagagagaacagtcacggactcggccagactaagtatgggcctgtctgaatccaaactcagctaagttccaaaagcataaagagagatgtgtaatgaaatgaacgtattctagaaacgaaagcaatgttatgctttgtttttgagccacatgtttttgggagatggagagaatcttttttacgtttttaacctaacccacttggcacttggccaaaaaagtgagaagaaactgtggcgaatgagtaggccacgccatggactttgttccttgtccttcaaaagttaaatttatgttatgcgtggggacaatctaagcaacgtggttcctttaaatatcgcagcttcctcttttacacttttggagcctacgtgttttgttttggaccggccaaatacacgagtcagtcagtttagaaataatttggatgtccaaaaatcttggagatccaaataaaataattagcatgttttagttcataagaatatgaaatgtagataaactgtctatattaatttttccatagaattggctttttatcgaggtgatgtacttaatgactttgttgattactactcgtataacaataaagaatatgatactatgtgagacttataatgaatttggtgtgtgttaattaatccagttgaaacagtttaataacaaatcagaataaaaattgtagtaagaaaatttgaacgctgatccttcaacctagatagtgaacctttcaaatactatatgattcacgtgtaatgtttttgaccgttggttatttttgtgtgaactatattaacttatcaatatcgaaaggctaaataagtaaataactaaaagaaagttcaggaaacaactcgacctaatgacctatcatttctgatcacccgtcctataaatacatacgtaagatcattcgttact

(19) United States
(12) Patent Application Publication
(10) Pub. No.: US 2010/0062137 A1
(43) Pub. Date: Mar. 11, 2010

(54) MODULATING PLANT TOCOPHEROL LEVELS

(76) Inventors: Steven Craig Bobzin, Malibu, CA (US); Boris Jankowski, Santa Monica, CA (US); Amr Saad Ragab, Carmel, IN (US); Joon-Hyun Park, Oak Park, CA (US); Jennifer E. Van Fleet, West Hills, CA (US)

Correspondence Address: FISH & RICHARDSON P.C., P.O. BOX 1022, MINNEAPOLIS, MN, 55440-1022, US

(21) Appl. No.: 12/088,569

(22) PCT Filed: Sep. 29, 2006
(86) PCT No.: PCT/US06/38526

§ 371 Date: Jul. 22, 2008

Related U.S. Application Data
(60) Provisional application No. 60/722,708, filed on Sep. 30, 2005.

(51) Int. Cl. A23L 1/36; A23L 1/212; C12N 5/10; C07H 21/04; C11B 1/00; C12N 15/82; A01H 5/00

(52) U.S. Cl. 426/629; 426/615; 435/419; 536/23.6; 554/9; 800/278; 800/287; 800/298

(57) ABSTRACT

Plants and plant cells having modulated levels of tocopherols and/or tocotrienols are described herein. Materials and methods for making plants and plant cells with modulated levels of tocopherols and/or tocotrienols are also described.

### CROSS-REFERENCE TO RELATED APPLICATIONS

This application claims priority under 35 U.S.C.§119 to U.S. Provisional Application No. 60/722,708, filed on Sep. 30, 2005, which is incorporated herein by reference in its entirety.

### INCORPORATION-BY-REFERENCE & TEXT

The material on the accompanying compact disc is hereby incorporated by reference into this application. The accompanying compact discs all contain one identical file, 11696-175WO1-Sequence.txt, which was created on Sep. 29, 2006. The file named 11696-175WO1-Sequence.txt is 415 KB. The file can be accessed using Microsoft Word on a computer that uses Windows OS.

### TECHNICAL FIELD

This document provides materials and methods related to plants having modulated (e.g., increased) levels of tocopherols (e.g., α-, β-, δ-, and/or γ-tocopherol) and tocotrienols (e.g., α-, β-, δ-, and/or γ-tocotrienol). For example, this document provides plants having increased tocopherol levels as well as materials and methods for making plants, plant tissues, seeds, and oils with modulated levels of tocopherols.

### BACKGROUND

Vitamin E is a strong antioxidant, which protects polyunsaturated fatty acids in membranes against degradation by reactive oxygen species such as ozone, singlet oxygen, peroxides, and hydroperoxides. Vitamin E is essential for the proper functioning of many different body systems in mammals. It is required by the nervous system to maintain many of the nerves in the body and the spinal cord in good working order. It is necessary for the normal production of red blood cells. It is essential for normal reproduction. It is required for the health of muscle cells and for the proper function of cells in the heart. Vitamin E may also help reduce the risks of atherosclerosis (the formation of fatty plaques on the walls of blood vessels that causes heart disease). Vitamin E cannot be produced in animals and thus represents an essential component of the human diet. Some food sources containing vitamin E include plant and seed oils, nuts, whole grains, and green leafy vegetables.

Vitamin E is comprised of two groups of molecules, tocopherols and tocotrienols. The four naturally occurring tocopherols, α-, β-, δ-, and γ-tocopherol, differ in the number and position of methyl substituents on the aromatic ring. Just as there are four natural tocopherols, there are also four natural tocotrienols, α-, β, δ- and γ-tocotrienol. The tocotrienols differ from the tocopherols in the moiety at the side chain or tail. Tocopherols have a saturated phytyl side chain, whereas tocotrienols have an unsaturated isoprenoid or farnesyl side chain possessing three double bonds. In plants, biosynthesis of tocopherols and tocotrienols is localized to the plastids of seeds and the chloroplasts of leaves.

The recommended dietary allowance (RDA) for vitamin E is about 15 mg per day for adults. Daily intake of vitamin E in excess of the RDA is associated with decreased risk of cardiovascular disease and some cancers, improved immune function, and slowing of the progression of a number of degenerative human conditions. It is quite difficult to obtain these therapeutic levels of vitamin E from the average diet.

### SUMMARY

This document provides methods and materials related to modulating tocopherol and/or tocotrienol levels in plants. For example, this document provides plants having increased levels of tocopherols, plant cells and seeds having the ability to grow into plants having increased levels of tocopherols, plant products (e.g., plant oils, food, foodstuffs, and animal feed) having increased levels of tocopherols, and methods for making such plants, plant cells, and plant products. Plants having the ability to produce increased levels of tocopherols can be used, for example, as food sources of tocopherols, or as sources of tocopherols for inclusion in nutritional supplements or cosmetics.

In one embodiment, a method of altering the level of a secondary metabolite in a plant is provided. The method can include introducing into a plant cell an exogenous nucleic acid including a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, where a tissue of a plant produced from the plant cell has a difference in the level of one or both of a tocopherol and a tocotrienol as compared to the corresponding level in tissue of a control plant that does not include the nucleic acid.

In another embodiment, a method of altering the level of a secondary metabolite in a plant is provided. The method can include introducing into a plant cell an exogenous nucleic acid including a nucleotide sequence encoding a polypeptide having 80% or greater sequence identity to an amino acid sequence selected from the group consisting of SEQ ID NOs:2-9, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, where a tissue of a plant produced from the plant cell has a difference in the level of one or both of a tocopherol and a tocotrienol as compared to the corresponding level in tissue of a control plant that does not include the nucleic acid.

In another embodiment, a method of altering the level of a secondary metabolite in a plant is provided. The method can include introducing into a plant cell an exogenous nucleic acid including a nucleotide sequence encoding a polypeptide having 80% or greater sequence identity to an amino acid sequence selected from the group consisting of SEQ ID NOs:2-5, SEQ ID NO:17, SEQ ID NO:19, SEQ ID NO:21, SEQ ID NO:23, SEQ ID NOs:25-26, SEQ ID NO:30, SEQ ID NOs:32-34, SEQ ID NO:36-37, SEQ ID NOs:48-49, SEQ ID NO:52, SEQ ID NO:54, SEQ ID NOs:57-58, SEQ ID NO:61, SEQ ID NO:64, SEQ ID NOs:71-72, SEQ ID NO:75, SEQ ID NO:77, SEQ ID NOs:83-84, SEQ ID NO:86, SEQ ID NO:88, SEQ ID NO:91, SEQ ID NO:93, SEQ ID NO:95, SEQ ID NO:97, SEQ ID NO:99, SEQ ID NO:101, and the consensus sequences set forth in FIGS. 7-13, where a tissue of a plant produced from the plant cell has a difference in the level of one or both of a tocopherol and a tocotrienol as compared to the corresponding level in tissue of a control plant that does not include the nucleic acid.

In a further embodiment, a method of altering the level of a secondary metabolite in a plant is provided. The method can include introducing into a plant cell an exogenous nucleic acid including a nucleotide sequence encoding a polypeptide having 80% or greater sequence identity to an amino acid sequence selected from the group consisting of SEQ ID NOs:2-5, SEQ ID NO:17, SEQ ID NO:19, SEQ ID NO:21, SEQ ID NO:23, SEQ ID NOs:25-26, SEQ ID NO:30, SEQ ID NOs:32-34, SEQ ID NO:36-37, SEQ ID NOs:48-49, SEQ ID NO:52, SEQ ID NO:54, SEQ ID NOs:57-58, SEQ ID NO:61, SEQ ID NO:64, SEQ ID NOs:71-72, SEQ ID NO:75, SEQ ID NO:77, SEQ ID NOs:83-84, SEQ ID NO:86, SEQ ID NO:88, SEQ ID NO:91, SEQ ID NO:93, SEQ ID NO:95, SEQ ID NO:97, SEQ ID NO:99, and SEQ ID NO:101. A sequence identity can be 85% or greater, 90% or greater, or 95% or greater. A nucleotide sequence can encode a polypeptide including an amino acid sequence corresponding to SEQ ID NO:2, SEQ ID NO:3, SEQ ID NO:25, SEQ ID NO:32, SEQ ID NO:48, SEQ ID NO:64, SEQ ID NO:77, or SEQ ID NO:88. A nucleotide sequence can encode a polypeptide including an amino acid sequence corresponding to the consensus sequence set forth in FIG. 7, FIG. 8, FIG. 9, FIG. 10, FIG. 11, FIG. 12, or FIG. 13. A difference can be an increase in the level of a tocopherol or a tocotrienol.

An exogenous nucleic acid can be operably linked to a regulatory region. The regulatory region can be a cell-specific or tissue-specific promoter, such as a seed-specific promoter. The seed-specific promoter can be the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, or the barley hordein gene promoter. The regulatory region can be a broadly expressing promoter, such as p326 (SEQ ID NO:178), YP0158 (SEQ ID NO:159), YP0214 (SEQ ID NO:163), YP0380 (SEQ ID NO:172), PT0848 (SEQ ID NO:128), PT0633 (SEQ ID NO:109), YP0050 (SEQ ID NO:137), YP0144 (SEQ ID NO:157), or YP0190 (SEQ ID NO:161). The regulatory region can be a constitutive promoter or an inducible promoter.

A plant can be from a genus selected from the group consisting of Acokanthera, Aesculus, Anamirta, Ananas, Arachis, Betula, Bixa, Brassica, Calendula, Carthamus, Centella, Chrysanthemum, Cinnamoinum, Citrullus, Coffea, Convallaria, Curcuma, Cymbopogon, Daphne, Elaeis, Euphorbia, Fragaria, Glycine, Glycyrrhiza, Gossypium, Helianthus, Isodon, Lactuca, Lavandula, Linum, Luffa, Lycopersicon, Mentha, Musa, Ocimum, Origanum, Oryza, Rabdosia, Ricinus, Rosmarinus, Ruscus, Salvia, Sesamum, Solanum, Strophanthus, Theobroma, Thymus, Triticum, Vitis, and Zea. A plant can be a species selected from Ananas comosus, Bixa orellana, Brassica campestris, Brassica napus, Brassica oleracea, Calendula officinalis, Chrysanthemum parthenium, Cinnamomum camphora, Coffea arabica, Glycine max, Glycyrrhiza glabra, Gossypium spp., Lactuca sativa, Lycopersicon esculentum, Mentha piperita, Mentha spicata, Musa paradisiaca, Oryza sativa, Rosmarinus officinalis, Solanum tuberosum, Theobroma cacao, Triticum aestivum, Vitis vinifera, and Zea mays.

A plant can be selected from the group consisting of peanut, safflower, flax, sugar beet, chick peas, alfalfa, spinach, clover, cabbage, lentils, mustard, soybean, lettuce, castor bean, sesame, carrot, grape, cotton, crambe, strawberry, amaranth, high erucic acid canola, broccoli, peas, pepper, tomato, potato, kidney beans, lima beans, dry beans, green beans, watermelon, cantaloupe, peach, pear, apple, cherry, orange, lemon, grapefruit, plum, mango, oilseed rape, sunflower, garlic, oil palm, date palm, banana, sweet corn, popcorn, field corn, wheat, rye, barley, oat, onion, pineapple, rice, millet, and sorghum. A tissue can be leaf tissue, seed tissue, or fruit tissue.

A method of producing a plant tissue is also provided. The method can include growing a plant cell including an exogenous nucleic acid including a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, where the tissue has a difference in the level of one or both of a tocopherol and a tocotrienol as compared to the corresponding level in tissue of a control plant that does not comprise the nucleic acid.

A method of producing a secondary metabolite is also provided. The method can include extracting a tocopherol or a tocotrienol from transgenic plant tissue including an exogenous nucleic acid including a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, where the tissue has a difference in the level of one or both of a tocopherol and a tocotrienol as compared to the corresponding level in tissue of a control plant that does not include the nucleic acid. A sequence identity can be 85% or greater, 90% or greater, or 95% or greater. A nucleotide sequence can encode a polypeptide corresponding to SEQ ID NO:2, SEQ ID NO:3, SEQ ID NO:25, SEQ ID NO:32, SEQ ID NO:48, SEQ ID NO:64, SEQ ID NO:77, or SEQ ID NO:88. A nucleotide sequence can encode a polypeptide corresponding to the consensus sequence set forth in any of FIGS. 7-13. A difference can be an increase in the level of a tocopherol or a tocotrienol.

An exogenous nucleic acid can be operably linked to a regulatory region. The regulatory region can be a cell-specific or tissue-specific promoter, such as a seed-specific promoter. The seed-specific promoter can be the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, or the barley hordein gene promoter. The regulatory region can be a broadly expressing promoter, such as p326 (SEQ ID NO:178), YP0158 (SEQ ID NO:159), YP0214 (SEQ ID NO:163), YP0380 (SEQ ID NO:172), PT0848 (SEQ ID NO:128), PT0633 (SEQ ID NO:109), YP0050 (SEQ ID NO:137), YP0144 (SEQ ID NO:157), and YP0190 (SEQ ID NO:161). The regulatory region can be a constitutive promoter or an inducible promoter. The regulatory regions can be cell-specific or tissue-specific promoters, such as seed-specific promoters. The regulatory regions can be broadly expressing promoters, constitutive promoters, or inducible promoters.

A plant can be from a genus selected from the group consisting of Acokanthera, Aesculus, Anamirta, Ananas, Arachis, Betula, Bixa, Brassica, Calendula, Carthamus, Centella, Chrysanthemum, Cinnamomum, Citrullus, Coffea, Convallaria, Curcuma, Cymbopogon, Daphne, Elaeis, Euphorbia, Fragaria, Glycine, Glycyrrhiza, Gossypium, Helianthus, Isodon, Lactuca, Lavandula, Linum, Luffa, Lycopersicon, Mentha, Musa, Ocimum, Origanum, Oryza, Rabdosia, Ricinus, Rosmarinus, Ruscus, Salvia, Sesamum, Solanum, Strophanthus, Theobroma, Thymus, Triticum, Vitis, and Zea. A plant can be a species selected from Ananas comosus, Bixa orellana, Brassica campestris, Brassica napus, Brassica oleracea, Calendula officinalis, Chrysanthemum parthenium, Cinnamomum camphora, Coffea arabica, Glycine max, Glycyrrhiza glabra, Gossypium spp., Lactuca sativa, Lycopersicon esculentum, Mentha piperita, Mentha spicata, Musa paradisiaca, Oryza sativa, Rosmarinus officinalis, Solanum tuberosum, Theobroma cacao, Triticum aestivum, Vitis vinifera, and Zea mays.

A plant can be selected from the group consisting of peanut, safflower, flax, sugar beet, chick peas, alfalfa, spinach, clover, cabbage, lentils, mustard, soybean, lettuce, castor bean, sesame, carrot, grape, cotton, crambe, strawberry, amaranth, high erucic acid canola, broccoli, peas, pepper, tomato, potato, kidney beans, lima beans, dry beans, green beans, watermelon, cantaloupe, peach, pear, apple, cherry, orange, lemon, grapefruit, plum, mango, oilseed rape, sunflower, garlic, oil palm, date palm, banana, sweet corn, popcorn, field corn, wheat, rye, barley, oat, onion, pineapple, rice, millet, and sorghum.

A tissue can be leaf tissue, seed tissue, fruit tissue, or a tissue culture.

A plant cell is also provided. The plant cell can include an exogenous nucleic acid including a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, where expression of the exogenous nucleic acid in tissue of a plant produced from the plant cell has a difference in the level of one or both of a tocopherol and a tocotrienol as compared to the corresponding level in tissue of a control plant that does not include the exogenous nucleic acid. A sequence identity can be 85% or greater, 90% or greater, or 95% or greater. A nucleotide sequence can encode a polypeptide including an amino acid sequence corresponding to SEQ ID NO:2, SEQ ID NO:3, SEQ ID NO:25, SEQ ID NO:32, SEQ ID NO:48, SEQ ID NO:64, SEQ ID NO:77, or SEQ ID NO:88. A nucleotide sequence can encode a polypeptide including an amino acid sequence corresponding to the consensus sequence set forth in any of FIGS. 7-13. A difference can be an increase in the level of a tocopherol or a tocotrienol.

An exogenous nucleic acid can be operably linked to a regulatory region. The regulatory region can be a cell-specific or tissue-specific promoter, such as a seed-specific promoter. The seed-specific promoter can be the napin promoter, the Arcelin-5 promoter, the phaseolin gene promoter, the soybean trypsin inhibitor promoter, the ACP promoter, the stearoyl-ACP desaturase gene, the soybean a' subunit of (3-conglycinin promoter, the oleosin promoter, the 15 kD zein promoter, the 1610 zein promoter, the 19 kD zein promoter, the 22 kD zein promoter, the 27 kD zein promoter, the Osgt-1 promoter, the beta-amylase gene promoter, or the barley hordein gene promoter. The regulatory region can be a broadly expressing promoter, such as p326 (SEQ ID NO:178), YP0158 (SEQ ID NO:159), YP0214 (SEQ ID NO:163), YP0380 (SEQ ID NO:172), PT0848 (SEQ ID NO:128), PT0633 (SEQ ID NO:109), YP0050 (SEQ ID NO:137), YP0144 (SEQ ID NO:157), and YP0190 (SEQ ID NO:161). The regulatory region can be a constitutive promoter or an inducible promoter.

A plant can be from a genus selected from the group consisting of Acokanthera, Aesculus, Anamirta, Ananas, Arachis, Betula, Bixa, Brassica, Calendula, Carthamus, Centella, Chrysanthemum, Cinnamomum, Citrullus, Coffea, Convallaria, Curcuma, Cymbopogon, Daphne, Elaeis, Euphorbia, Fragaria, Glycine, Glycyrrhiza, Gossypium, Helianthus, Isodon, Lactuca, Lavandula, Linum, Luffa, Lycopersicon, Mentha, Musa, Ocimum, Origanum, Oryza, Rabdosia, Ricinus, Rosmarinus, Ruscus, Salvia, Sesamum, Solanum, Strophanthus, Theobroina, Thymus, Triticum, Vitis, and Zea. A plant can be a species selected from Ananas comosus, Bixa orellana, Brassica campestris, Brassica napus, Brassica oleracea, Calendula officinalis, Chrysanthemum parthenium, Cinnamomum camphora, Coffea arabica, Glycine max, Glycyrrhiza glabra, Gossypium spp., Lactuca sativa, Lycopersicon esculentum, Mentha piperita, Mentha spicata, Musa paradisiaca, Oryza sativa, Rosmarinus officinalis, Solanum tuberosum, Theobroina cacao, Triticum aestivum, Vitis vinifera, and Zea mays. A plant can be selected from the group consisting of peanut, safflower, flax, sugar beet, chick peas, alfalfa, spinach, clover, cabbage, lentils, mustard, soybean, lettuce, castor bean, sesame, carrot, grape, cotton, crambe, strawberry, amaranth, high erucic acid canola, broccoli, peas, pepper, tomato, potato, kidney beans, lima beans, dry beans, green beans, watermelon, cantaloupe, peach, pear, apple, cherry, orange, lemon, grapefruit, plum, mango, oilseed rape, sunflower, garlic, oil palm, date palm, banana, sweet corn, popcorn, field corn, wheat, rye, barley, oat, onion, pineapple, rice, millet, and sorghum. A tissue can be leaf tissue, seed tissue, or fruit tissue.

Tocopherol-modulating polypeptides are provided herein. A tocopherol-modulating polypeptide can be a polypeptide including the amino acid sequence corresponding to SEQ ID NO:2. A tocopherol-modulating polypeptide can be a polypeptide including the amino acid sequence corresponding to SEQ ID NO:3. A tocopherol-modulating polypeptide can be a polypeptide including the amino acid sequence corresponding to SEQ ID NO:4. A tocopherol-modulating polypeptide can be a polypeptide including the amino acid sequence corresponding to SEQ ID NO:5. A tocopherol-modulating polypeptide can be a polypeptide including the amino acid sequence corresponding to SEQ ID NO:6. A tocopherol-modulating polypeptide can be a polypeptide including the amino acid sequence corresponding to SEQ ID NO:7. A tocopherol-modulating polypeptide can be a polypeptide including the amino acid sequence corresponding to SEQ ID NO:8. A tocopherol-modulating polypeptide can be a polypeptide including the amino acid sequence corresponding to SEQ ID NO:9. A tocopherol-modulating polypeptide can be a polypeptide including the amino acid sequence corresponding to SEQ ID NO:10. A tocopherol-modulating polypeptide can be a polypeptide including the amino acid sequence corresponding to SEQ ID NO:11. A tocopherol-modulating polypeptide can be a polypeptide including the amino acid sequence corresponding to SEQ ID NO:12. A tocopherol-modulating polypeptide can be a polypeptide including the amino acid sequence corresponding to SEQ ID NO:13. A tocopherol-modulating polypeptide can be a polypeptide including the amino acid sequence corresponding to SEQ ID NO:14. A tocopherol-modulating polypeptide can be a polypeptide including the amino acid sequence corresponding to SEQ ID NO:15. A tocopherol-modulating polypeptide can be a polypeptide including the amino acid sequence corresponding to SEQ ID NO:17. A tocopherol-modulating polypeptide can be a polypeptide including the amino acid sequence corresponding to SEQ ID NO:19. A tocopherol-modulating polypeptide can be a polypeptide including the amino acid sequence corresponding to SEQ ID NO:21. A tocopherol-modulating polypeptide can be a polypeptide including the amino acid sequence corresponding to SEQ ID NO:23. A tocopherol-modulating polypeptide can be a polypeptide including the amino acid sequence corresponding to SEQ ID NO:25. A tocopherol-modulating polypeptide can be a polypeptide including the amino acid sequence corresponding to SEQ ID NO:26. A tocopherol-modulating polypeptide can be a polypeptide including the amino acid sequence corresponding to SEQ ID NO:27. A tocopherol-modulating polypeptide can be a polypeptide including the amino acid sequence corresponding to SEQ ID NO:28. A tocopherol-modulating polypeptide can be a polypeptide including the amino acid sequence corresponding to SEQ ID NO:29. A tocopherol-modulating polypeptide can be a polypeptide including the amino acid sequence corresponding to SEQ ID NO:30. A tocopherol-modulating polypeptide can be a polypeptide including the amino acid sequence corresponding to SEQ ID NO:32. A tocopherol-modulating polypeptide can be a polypeptide including the amino acid sequence corresponding to SEQ ID NO:33. A tocopherol-modulating polypeptide can be a polypeptide including the amino acid sequence corresponding to SEQ ID NO:34. A tocopherol-modulating polypeptide can be a polypeptide including the amino acid sequence corresponding to SEQ ID NO:35. A tocopherol-modulating polypeptide can be a polypeptide including the amino acid sequence corresponding to SEQ ID NO:36. A tocopherol-modulating polypeptide can be a polypeptide including the amino acid sequence corresponding to SEQ ID NO:37. A tocopherol-modulating polypeptide can be a polypeptide including the amino acid sequence corresponding to SEQ ID NO:38. A tocopherol-modulating polypeptide can be a polypeptide including the amino acid sequence corresponding to SEQ ID NO:39. A tocopherol-modulating polypeptide can be a polypeptide including the amino acid sequence corresponding to SEQ ID NO:40. A tocopherol-modulating polypeptide can be a polypeptide including the amino acid sequence corresponding to SEQ ID NO:41. A tocopherol-modulating polypeptide can be a polypeptide including the amino acid sequence corresponding to SEQ ID NO:42. A tocopherol-modulating polypeptide can be a polypeptide including the amino acid sequence corresponding to SEQ ID NO:43. A tocopherol-modulating polypeptide can be a polypeptide including the amino acid sequence corresponding to SEQ ID NO:44. A tocopherol-modulating polypeptide can be a polypeptide including the amino acid sequence corresponding to SEQ ID NO:45. A tocopherol-modulating polypeptide can be a polypeptide including the amino acid sequence corresponding to SEQ ID NO:46. A tocopherol-modulating polypeptide can be a polypeptide including the amino acid sequence corresponding to SEQ ID NO:48. A tocopherol-modulating polypeptide can be a polypeptide including the amino acid sequence corresponding to SEQ ID NO:49. A tocopherol-modulating polypeptide can be a polypeptide including the amino acid sequence corresponding to SEQ ID NO:50. A tocopherol-modulating polypeptide can be a polypeptide including the amino acid sequence corresponding to SEQ ID NO:52. A tocopherol-modulating polypeptide can be a polypeptide including the amino acid sequence corresponding to SEQ ID NO:53. A tocopherol-modulating polypeptide can be a polypeptide including the amino acid sequence corresponding to SEQ ID NO:54. A tocopherol-modulating polypeptide can be a polypeptide including the amino acid sequence corresponding to SEQ ID NO:55. A tocopherol-modulating polypeptide can be a polypeptide including the amino acid sequence corresponding to SEQ ID NO:57. A tocopherol-modulating polypeptide can be a polypeptide including the amino acid sequence corresponding to SEQ ID NO:58. A tocopherol-modulating polypeptide can be a polypeptide including the amino acid sequence corresponding to SEQ ID NO:59. A tocopherol-modulating polypeptide can be a polypeptide including the amino acid sequence corresponding to SEQ ID NO:60. A tocopherol-modulating polypeptide can be a polypeptide including the amino acid sequence corresponding to SEQ ID NO:61. A tocopherol-modulating polypeptide can be a polypeptide including the amino acid sequence corresponding to SEQ ID NO:62. A tocopherol-modulating polypeptide can be a polypeptide including the amino acid sequence corresponding to SEQ ID NO:64. A tocopherol-modulating polypeptide can be a polypeptide including the amino acid sequence corresponding to SEQ ID NO:65. A tocopherol-modulating polypeptide can be a polypeptide including the amino acid sequence corresponding to SEQ ID NO:66. A tocopherol-modulating polypeptide can be a polypeptide including the amino acid sequence corresponding to SEQ ID NO:67. A tocopherol-modulating polypeptide can be a polypeptide including the amino acid sequence corresponding to SEQ ID NO:68. A tocopherol-modulating polypeptide can be a polypeptide including the amino acid sequence corresponding to SEQ ID NO:69. A tocopherol-modulating polypeptide can be a polypeptide including the amino acid sequence corresponding to SEQ ID NO:71. A tocopherol-modulating polypeptide can be a polypeptide including the amino acid sequence corresponding to SEQ ID NO:72. A tocopherol-modulating polypeptide can be a polypeptide including the amino acid sequence corresponding to SEQ ID NO:73. A tocopherol-modulating polypeptide can be a polypeptide including the amino acid sequence corresponding to SEQ ID NO:75. A tocopherol-modulating polypeptide can be a polypeptide including the amino acid sequence corresponding to SEQ ID NO:77. A tocopherol-modulating polypeptide can be a polypeptide including the amino acid sequence corresponding to SEQ ID NO:78. A tocopherol-modulating polypeptide can be a polypeptide including the amino acid sequence corresponding to SEQ ID NO:79. A tocopherol-modulating polypeptide can be a polypeptide including the amino acid sequence corresponding to SEQ ID NO:80. A tocopherol-modulating polypeptide can be a polypeptide including the amino acid sequence corresponding to SEQ ID NO:81. A tocopherol-modulating polypeptide can be a polypeptide including the amino acid sequence corresponding to SEQ ID NO:82. A tocopherol-modulating polypeptide can be a polypeptide including the amino acid sequence corresponding to SEQ ID NO:83. A tocopherol-modulating polypeptide can be a polypeptide including the amino acid sequence corresponding to SEQ ID NO:84. A tocopherol-modulating polypeptide can be a polypeptide including the amino acid sequence corresponding to SEQ ID NO:85. A tocopherol-modulating polypeptide can be a polypeptide including the amino acid sequence corresponding to SEQ ID NO:86. A tocopherol-modulating polypeptide can be a polypeptide including the amino acid sequence corresponding to SEQ ID NO:88. A tocopherol-modulating polypeptide can be a polypeptide including the amino acid sequence corresponding to SEQ ID NO:89. A tocopherol-modulating polypeptide can be a polypeptide including the amino acid sequence corresponding to SEQ ID NO:90. A tocopherol-modulating polypeptide can be a polypeptide including the amino acid sequence corresponding to SEQ ID NO:91. A tocopherol-modulating polypeptide can be a polypeptide including the amino acid sequence corresponding to SEQ ID NO:93. A tocopherol-modulating polypeptide can be a polypeptide including the amino acid sequence corresponding to SEQ ID NO:94. A tocopherol-modulating polypeptide can be a polypeptide including the amino acid sequence corresponding to SEQ ID NO:95. A tocopherol-modulating polypeptide can be a polypeptide including the amino acid sequence corresponding to SEQ ID NO:97. A tocopherol-modulating polypeptide can be a polypeptide including the amino acid sequence corresponding to SEQ ID NO:98. A tocopherol-modulating polypeptide can be a polypeptide including the amino acid sequence corresponding to SEQ ID NO:99. A tocopherol-modulating polypeptide can be a polypeptide including the amino acid sequence corresponding to SEQ ID NO:101. A tocopherol-modulating polypeptide can be a polypeptide including the amino acid sequence corresponding to SEQ ID NO:102. A tocopherol-modulating polypeptide can be a polypeptide including the amino acid sequence corresponding to the consensus sequence set forth in FIG. 7. A tocopherol-modulating polypeptide can be a polypeptide including the amino acid sequence corresponding to the consensus sequence set forth in FIG. 8. A tocopherol-modulating polypeptide can be a polypeptide including the amino acid sequence corresponding to the consensus sequence set forth in FIG. 9. A tocopherol-modulating polypeptide can be a polypeptide including the amino acid sequence corresponding to the consensus sequence set forth in FIG. 10. A tocopherol-modulating polypeptide can be a polypeptide including the amino acid sequence corresponding to the consensus sequence set forth in FIG. 11. A tocopherol-modulating polypeptide can be a polypeptide including the amino acid sequence corresponding to the consensus sequence set forth in FIG. 12. A tocopherol-modulating polypeptide can be a polypeptide including the amino acid sequence corresponding to the consensus sequence set forth in FIG. 13.

A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:2. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:3. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:4. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:5. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:6. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:7. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:8. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:9. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:10. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:11. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:12. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:13. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:14. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:15. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:17. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:19. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:21. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:23. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:25. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:26. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:27. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:28. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:29. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:30. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:32. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:33. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:34. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:35. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:36. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:37. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:38. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:39. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:40. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:41. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:42. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:43. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:44. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:45. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:46. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:48. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:49. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:50. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:52. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:53. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ED NO:54. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:55. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:57. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:58. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:59. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:60. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:61. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:62. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:64. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:65. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:66. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:67. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:68. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:69. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:71. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:72. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:73. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:75. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:77. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:78. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:79. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:80. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:81. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:82. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:83. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:84. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:85. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:86. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:88. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:89. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:90. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:91. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:93. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:94. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:95. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:97. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:98. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:99. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:101. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:102. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 7. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 8. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 9. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 10. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 11. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 12. A tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 13.

Nucleic acids encoding tocopherol-modulating polypeptides are provided herein. Such nucleic acids can be used to transform plant cells. A nucleic acid encoding a polypeptide that includes an amino acid sequence corresponding to SEQ ID NO:2 can be used to transform a plant cell. A nucleic acid encoding a polypeptide that includes an amino acid sequence corresponding to SEQ ID NO:3 can be used to transform a plant cell. A nucleic acid encoding a polypeptide that includes an amino acid sequence corresponding to SEQ ID NO:4 can be used to transform a plant cell. A nucleic acid encoding a polypeptide that includes an amino acid sequence corresponding to SEQ ID NO:5 can be used to transform a plant cell. A nucleic acid encoding a polypeptide that includes an amino acid sequence corresponding to SEQ ID NO:6 can be used to transform a plant cell. A nucleic acid encoding a polypeptide that includes an amino acid sequence corresponding to SEQ ID NO:7 can be used to transform a plant cell. A nucleic acid encoding a polypeptide that includes an amino acid sequence corresponding to SEQ ID NO:8 can be used to transform a plant cell. A nucleic acid encoding a polypeptide that includes an amino acid sequence corresponding to SEQ ID NO:9 can be used to transform a plant cell. A nucleic acid encoding a polypeptide that includes an amino acid sequence corresponding to SEQ ID NO:10 can be used to transform a plant cell. A nucleic acid encoding a polypeptide that includes an amino acid sequence corresponding to SEQ ID NO:11 can be used to transform a plant cell, A nucleic acid encoding a polypeptide that includes an amino acid sequence corresponding to SEQ ID NO:12 can be used to transform a plant cell. A nucleic acid encoding a polypeptide that includes an amino acid sequence corresponding to SEQ ID NO:13 can be used to transform a plant cell. A nucleic acid encoding a polypeptide that includes an amino acid sequence corresponding to SEQ ID NO:14 can be used to transform a plant cell. A nucleic acid encoding a polypeptide that includes an amino acid sequence corresponding to SEQ ID NO:15 can be used to transform a plant cell. A nucleic acid encoding a polypeptide that includes an amino acid sequence corresponding to SEQ ID NO:17 can be used to transform a plant cell. A nucleic acid encoding a polypeptide that includes an amino acid sequence corresponding to SEQ ID NO:19 can be used to transform a plant cell. A nucleic acid encoding a polypeptide that includes an amino acid sequence corresponding to SEQ ID NO:21 can be used to transform a plant cell. A nucleic acid encoding a polypeptide that includes an amino acid sequence corresponding to SEQ ID NO:23 can be used to transform a plant cell. A nucleic acid encoding a polypeptide that includes an amino acid sequence corresponding to SEQ ID NO:25 can be used to transform a plant cell. A nucleic acid encoding a polypeptide that includes an amino acid sequence corresponding to SEQ ID NO:26 can be used to transform a plant cell. A nucleic acid encoding a polypeptide that includes an amino acid sequence corresponding to SEQ ID NO:27 can be used to transform a plant cell. A nucleic acid encoding a polypeptide that includes an amino acid sequence corresponding to SEQ ID NO:28 can be used to transform a plant cell. A nucleic acid encoding a polypeptide that includes an amino acid sequence corresponding to SEQ ID NO:29 can be used to transform a plant cell. A nucleic acid encoding a polypeptide that includes an amino acid sequence corresponding to SEQ ID NO:30 can be used to transform a plant cell. A nucleic acid encoding a polypeptide that includes an amino acid sequence corresponding to SEQ ID NO:32 can be used to transform a plant cell. A nucleic acid encoding a polypeptide that includes an amino acid sequence corresponding to SEQ ID NO:33 can be used to transform a plant cell. A nucleic acid encoding a polypeptide that includes an amino acid sequence corresponding to SEQ ID NO:34 can be used to transform a plant cell. A nucleic acid encoding a polypeptide that includes an amino acid sequence corresponding to SEQ ID NO:35 can be used to transform a plant cell. A nucleic acid encoding a polypeptide that includes an amino acid sequence corresponding to SEQ ID NO:36 can be used to transform a plant cell. A nucleic acid encoding a polypeptide that includes an amino acid sequence corresponding to SEQ ID NO:37 can be used to transform a plant cell. A nucleic acid encoding a polypeptide that includes an amino acid sequence corresponding to SEQ ID NO:38 can be used to transform a plant cell. A nucleic acid encoding a polypeptide that includes an amino acid sequence corresponding to SEQ ID NO:39 can be used to transform a plant cell. A nucleic acid encoding a polypeptide that includes an amino acid sequence corresponding to SEQ ID NO:40 can be used to transform a plant cell. A nucleic acid encoding a polypeptide that includes an amino acid sequence corresponding to SEQ ID NO:41 can be used to transform a plant cell. A nucleic acid encoding a polypeptide that includes an amino acid sequence corresponding to SEQ ID NO:42 can be used to transform a plant cell. A nucleic acid encoding a polypeptide that includes an amino acid sequence corresponding to SEQ ID NO:43 can be used to transform a plant cell. A nucleic acid encoding a polypeptide that includes an amino acid sequence corresponding to SEQ ID NO:44 can be used to transform a plant cell. A nucleic acid encoding a polypeptide that includes an amino acid sequence corresponding to SEQ ID NO:45 can be used to transform a plant cell. A nucleic acid encoding a polypeptide that includes an amino acid sequence corresponding to SEQ ID NO:46 can be used to transform a plant cell. A nucleic acid encoding a polypeptide that includes an amino acid sequence corresponding to SEQ ID NO:48 can be used to transform a plant cell, A nucleic acid encoding a polypeptide that includes an amino acid sequence corresponding to SEQ ID NO:49 can be used to transform a plant cell. A nucleic acid encoding a polypeptide that includes an amino acid sequence corresponding to SEQ ID NO:50 can be used to transform a plant cell. A nucleic acid encoding a polypeptide that includes an amino acid sequence corresponding to SEQ ID NO:52 can be used to transform a plant cell. A nucleic acid encoding a polypeptide that includes an amino acid sequence corresponding to SEQ ID NO:53 can be used to transform a plant cell. A nucleic acid encoding a polypeptide that includes an amino acid sequence corresponding to SEQ ID NO:54 can be used to transform a plant cell. A nucleic acid encoding a polypeptide that includes an amino acid sequence corresponding to SEQ ID NO:55 can be used to transform a plant cell. A nucleic acid encoding a polypeptide that includes an amino acid sequence corresponding to SEQ ID NO:57 can be used to transform a plant cell. A nucleic acid encoding a polypeptide that includes an amino acid sequence corresponding to SEQ ID NO:58 can be used to transform a plant cell. A nucleic acid encoding a polypeptide that includes an amino acid sequence corresponding to SEQ ID NO:59 can be used to transform a plant cell. A nucleic acid encoding a polypeptide that includes an amino acid sequence corresponding to SEQ ID NO:60 can be used to transform a plant cell. A nucleic acid encoding a polypeptide that includes an amino acid sequence corresponding to SEQ ID NO:61 can be used to transform a plant cell. A nucleic acid encoding a polypeptide that includes an amino acid sequence corresponding to SEQ ID NO:62 can be used to transform a plant cell. A nucleic acid encoding a polypeptide that includes an amino acid sequence corresponding to SEQ ID NO:64 can be used to transform a plant cell. A nucleic acid encoding a polypeptide that includes an amino acid sequence corresponding to SEQ ID NO:65 can be used to transform a plant cell. A nucleic acid encoding a polypeptide that includes an amino acid sequence corresponding to SEQ ID NO:66 can be used to transform a plant cell. A nucleic acid encoding a polypeptide that includes an amino acid sequence corresponding to SEQ ID NO:67 can be used to transform a plant cell. A nucleic acid encoding a polypeptide that includes an amino acid sequence corresponding to SEQ ID NO:68 can be used to transform a plant cell. A nucleic acid encoding a polypeptide that includes an amino acid sequence corresponding to SEQ ID NO:69 can be used to transform a plant cell. A nucleic acid encoding a polypeptide that includes an amino acid sequence corresponding to SEQ ID NO:71 can be used to transform a plant cell. A nucleic acid encoding a polypeptide that includes an amino acid sequence corresponding to SEQ ID NO:72 can be used to transform a plant cell. A nucleic acid encoding a polypeptide that includes an amino acid sequence corresponding to SEQ ID NO:73 can be used to transform a plant cell. A nucleic acid encoding a polypeptide that includes an amino acid sequence corresponding to SEQ ID NO:75 can be used to transform a plant cell. A nucleic acid encoding a polypeptide that includes an amino acid sequence corresponding to SEQ ID NO:77 can be used to transform a plant cell. A nucleic acid encoding a polypeptide that includes an amino acid sequence corresponding to SEQ ID NO:78 can be used to transform a plant cell. A nucleic acid encoding a polypeptide that includes an amino acid sequence corresponding to SEQ ID NO:79 can be used to transform a plant cell. A nucleic acid encoding a polypeptide that includes an amino acid sequence corresponding to SEQ ID NO:80 can be used to transform a plant cell. A nucleic acid encoding a polypeptide that includes an amino acid sequence corresponding to SEQ ID NO:81 can be used to transform a plant cell. A nucleic acid encoding a polypeptide that includes an amino acid sequence corresponding to SEQ ID NO:82 can be used to transform a plant cell. A nucleic acid encoding a polypeptide that includes an amino acid sequence corresponding to SEQ ID NO:83 can be used to transform a plant cell. A nucleic acid encoding a polypeptide that includes an amino acid sequence corresponding to SEQ ID NO:84 can be used to transform a plant cell. A nucleic acid encoding a polypeptide that includes an amino acid sequence corresponding to SEQ ID NO:85 can be used to transform a plant cell. A nucleic acid encoding a polypeptide that includes an amino acid sequence corresponding to SEQ ID NO:86 can be used to transform a plant cell. A nucleic acid encoding a polypeptide that includes an amino acid sequence corresponding to SEQ ID NO:88 can be used to transform a plant cell. A nucleic acid encoding a polypeptide that includes an amino acid sequence corresponding to SEQ ID NO:89 can be used to transform a plant cell. A nucleic acid encoding a polypeptide that includes an amino acid sequence corresponding to SEQ ID NO:90 can be used to transform a plant cell. A nucleic acid encoding a polypeptide that includes an amino acid sequence corresponding to SEQ ID NO:91 can be used to transform a plant cell. A nucleic acid encoding a polypeptide that includes an amino acid sequence corresponding to SEQ ID NO:93 can be used to transform a plant cell. A nucleic acid encoding a polypeptide that includes an amino acid sequence corresponding to SEQ ID NO:94 can be used to transform a plant cell. A nucleic acid encoding a polypeptide that includes an amino acid sequence corresponding to SEQ ID NO:95 can be used to transform a plant cell. A nucleic acid encoding a polypeptide that includes an amino acid sequence corresponding to SEQ ID NO:97 can be used to transform a plant cell. A nucleic acid encoding a polypeptide that includes an amino acid sequence corresponding to SEQ ID NO:98 can be used to transform a plant cell. A nucleic acid encoding a polypeptide that includes an amino acid sequence corresponding to SEQ ID NO:99 can be used to transform a plant cell. A nucleic acid encoding a polypeptide that includes an amino acid sequence corresponding to SEQ ID NO:101 can be used to transform a plant cell. A nucleic acid encoding a polypeptide that includes an amino acid sequence corresponding to SEQ ID NO:102 can be used to transform a plant cell. A nucleic acid encoding a polypeptide that includes an amino acid sequence corresponding to the consensus sequence set forth in FIG. 7 can be used to transform a plant cell. A nucleic acid encoding a polypeptide that includes an amino acid sequence corresponding to the consensus sequence set forth in FIG. 8 can be used to transform a plant cell. A nucleic acid encoding a polypeptide that includes an amino acid sequence corresponding to the consensus sequence set forth in FIG. 9 can be used to transform a plant cell. A nucleic acid encoding a polypeptide that includes an amino acid sequence corresponding to the consensus sequence set forth in FIG. 10 can be used to transform a plant cell. A nucleic acid encoding a polypeptide that includes an amino acid sequence corresponding to the consensus sequence set forth in FIG. 11 can be used to transform a plant cell. A nucleic acid encoding a polypeptide that includes an amino acid sequence corresponding to the consensus sequence set forth in FIG. 12 can be used to transform a plant cell. A nucleic acid encoding a polypeptide that includes an amino acid sequence corresponding to the consensus sequence set forth in FIG. 13 can be used to transform a plant cell.

A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:2 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:3 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:4 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:5 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:6 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:7 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:8 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:9 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:10 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:11 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:12 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:13 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:14 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:15 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:17 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:19 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:21 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:23 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:25 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:26 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:27 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:28 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:29 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:30 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:32 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:33 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:34 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:35 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:36 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:37 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:38 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:39 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:40 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:41 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:42 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:43 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:44 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:45 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:46 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:48 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:49 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:50 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:52 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:53 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:54 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:55 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:57 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:58 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:59 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:60 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:61 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:62 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:64 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:65 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:66 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:67 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:68 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:69 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:71 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:72 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:73 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:75 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:77 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:78 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:79 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:80 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:81 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:82 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:83 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:84 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:85 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:86 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:88 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:89 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:90 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:91 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:93 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:94 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:95 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:97 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:98 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:99 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:101 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:102 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 7 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 8 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 9 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 10 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 11 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 12 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to the consensus sequence set forth in FIG. 13 can be used to transform a plant cell.

One aspect of the invention is a plant comprising an exogenous nucleic acid comprising a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13. One or more tissues of the plant have a difference in the level of one or both of a tocopherol and a tocotrienol as compared to the corresponding level in tissue of a control plant that does not comprise the nucleic acid. Another aspect of the invention is a plant comprising at least two nucleotide sequences, wherein each nucleotide sequence encodes a polypeptide having 80% or greater sequence identity to an amino acid sequence selected from the group consisting of:

(a) SEQ ID NOs:2-15, SEQ ID NO:17, SEQ ID NO:19, SEQ ID NO:21, SEQ ID NO:23, and the consensus sequence set forth in FIG. 7;

(b) SEQ ID NOs:25-30 and the consensus sequence set forth in FIG. 8;

(c) SEQ ID NOs:32-46 and the consensus sequence set forth in FIG. 9;

(d) SEQ ID NOs:48-50, SEQ ID NOs:52-55, SEQ ID NOs:57-62, and the consensus sequence set forth in FIG. 10;

(e) SEQ ID NOs:64-69, SEQ ID NOs:71-73, SEQ ID NO:75, and the consensus sequence set forth in FIG. 11;

(f) SEQ ID NOs:77-86 and the consensus sequence set forth in FIG. 12; and

(g) SEQ ID NOs:88-91, SEQ ID NOs:93-95, SEQ ID NOs:97-99, SEQ ID NOs:101-102, and the consensus sequence set forth in FIG. 13. Each of the at least two nucleotide sequences is from a different one of (a), (b), (c), (d), (e), (f), or (g). One or more tissues of the plant have a difference in the level of one or both of a tocopherol and a tocotrienol as compared to the corresponding level in tissue of a control plant that does not comprise the at least two nucleotide sequences. Methods of making such plants are also provided. Such a method can comprise the steps of obtaining a plurality of plants transformed with an exogenous nucleic acid, the exogenous nucleic acid comprising a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, the nucleotide sequence being operably linked to a regulatory region; and selecting from among the plurality of plants at least one plant in which one or more tissues of the plant have a difference in the level of one or both of a tocopherol and a tocotrienol as compared to the corresponding level in tissue of a control plant that does not comprise the nucleic acid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is the nucleotide sequence of Ceres clone 19143 (SEQ ID NO:1).

FIG. 2 is the amino acid sequence encoded by Ceres clone 19143 (SEQ ID NO:2).

FIG. 3 is the nucleotide sequence of Ceres clone 92102 (SEQ ID NO:24).

FIG. 4 is the amino acid sequence encoded by Ceres clone 92102 (SEQ ID NO:25).

FIG. 5 is the nucleotide sequence of Ceres cDNA 23495742 (SEQ ID NO:31).

FIG. 6 is the amino acid sequence encoded by Ceres cDNA 23495742 (SEQ ID NO:32).

FIG. 7 is an alignment of SEQ ID NO:2 with orthologous amino acid sequences SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:14, and SEQ ID NO:15. The consensus sequence determined by the alignment is set forth.

FIG. 8 is an alignment of SEQ ID NO:25 with orthologous amino acid sequences SEQ ID NO:26, SEQ ID NO:27, SEQ ID NO:28, SEQ ID NO:29 and SEQ ID NO:30. The consensus sequence determined by the alignment is set forth.

FIG. 9 is an alignment of SEQ ID NO:32 with orthologous amino acid sequences SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:44, and SEQ ID NO:46. The consensus sequence determined by the alignment is set forth.

FIG. 10 is an alignment of SEQ ID NO:48 with orthologous amino acid sequences SEQ ID NO:49, SEQ ID NO:50, SEQ ID NO:52, SEQ ID NO:53, SEQ ID NO:54, SEQ ID NO:55, SEQ ID NO:57, SEQ ID NO:59, SEQ ID NO:60, and SEQ ID NO:61. The consensus sequence determined by the alignment is set forth.

FIG. 11 is an alignment of SEQ ID NO:64 with orthologous amino acid sequences SEQ ID NO:65, SEQ ID NO:66, SEQ ID NO:67, SEQ ID NO:69, SEQ ID NO:71, SEQ ID NO:72, and SEQ ID NO:75. The consensus sequence determined by the alignment is set forth.

FIG. 12 is an alignment of SEQ ID NO:77 with orthologous amino acid sequences SEQ ID NO:78, SEQ ID NO:79, SEQ ID NO:80, SEQ ID NO:82, SEQ ID NO:83, SEQ ID NO:84, and SEQ ID NO:86. The consensus sequence determined by the alignment is set forth.

FIG. 13 is an alignment of SEQ ID NO:88 with orthologous amino acid sequences SEQ ID NO:91, SEQ ID NO:93, SEQ ID NO:94, SEQ ID NO:97, and SEQ ID NO:101. The consensus sequence determined by the alignment is set forth.

### DETAILED DESCRIPTION

The materials and methods provided herein can be used to make plants, plant tissues, and plant products having modulated levels of tocopherols (e.g., α-, β-, δ-, and/or γ-tocopherol) and/or tocotrienols (e.g., α-, β-, δ-, and/or γ-tocotrienol). For example, plants having seeds and/or non-seed tissues with increased levels of tocopherols are provided herein. The methods can include introducing into a plant cell one or more nucleic acids that encode tocopherol-modulating polypeptides, wherein expression of the one or more polypeptides results in modulated levels (e.g., increased or decreased levels) of one or more tocopherols and/or tocotrienols. Plants and plant materials (e.g., seeds, non-seed tissues) produced using such methods can be used as food sources of tocopherols and/or tocotrienols, or as sources of tocopherols and/or tocotrienols for inclusion in nutritional supplements or cosmetics, for example.

### Polypeptides

Isolated polypeptides, including tocopherol-modulating polypeptides, are provided herein. The term “polypeptide” as used herein refers to a compound of two or more subunit amino acids, amino acid analogs, or other peptidomimetics, regardless of post-translational modification (e.g., phosphorylation or glycosylation). The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

By “isolated” or “purified” with respect to a polypeptide it is meant that the polypeptide is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

Described herein are tocopherol-modulating polypeptides. A tocopherol-modulating polypeptide can be effective to modulate a level of one or more tocopherols when expressed in a plant cell. For example, a tocopherol-modulating polypeptide can modulate tocopherol biosynthesis, stability, and/or degradation. A tocopherol-modulating polypeptide can also be effective to modulate a level of one or more tocotrienols by any mechanism when expressed in a plant cell. For example, a tocopherol-modulating polypeptide can modulate tocotrienol biosynthesis, stability, and/or degradation.

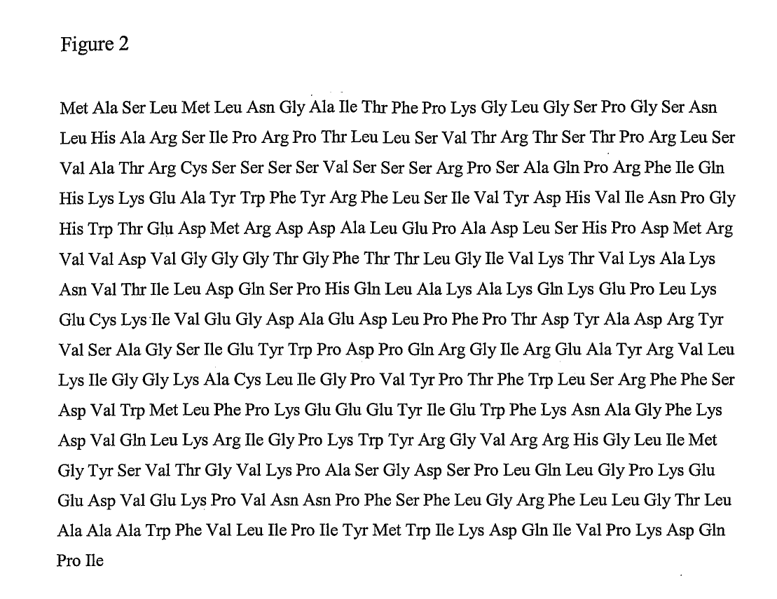

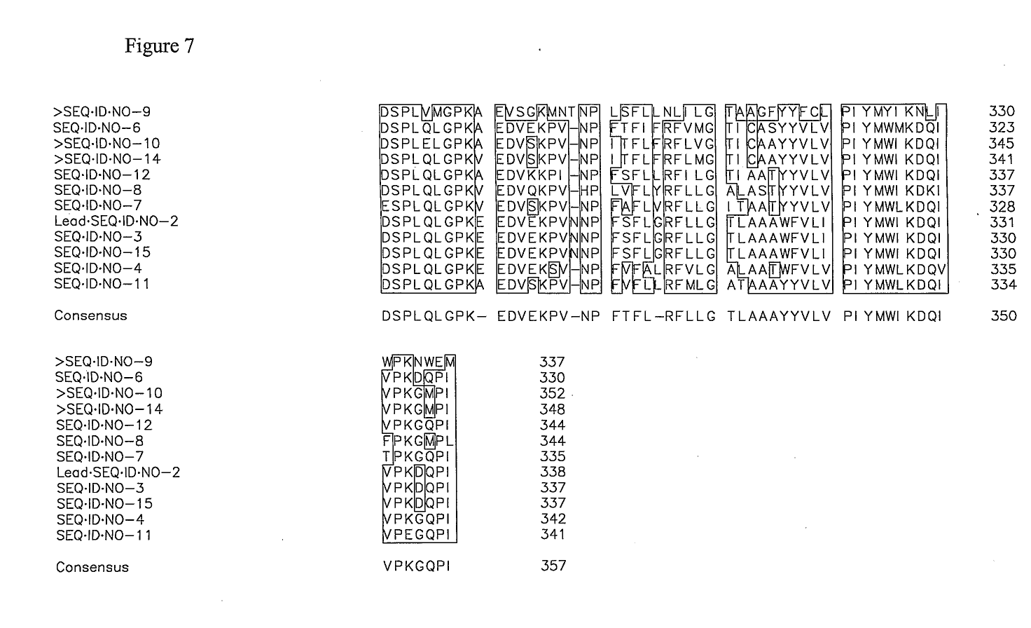

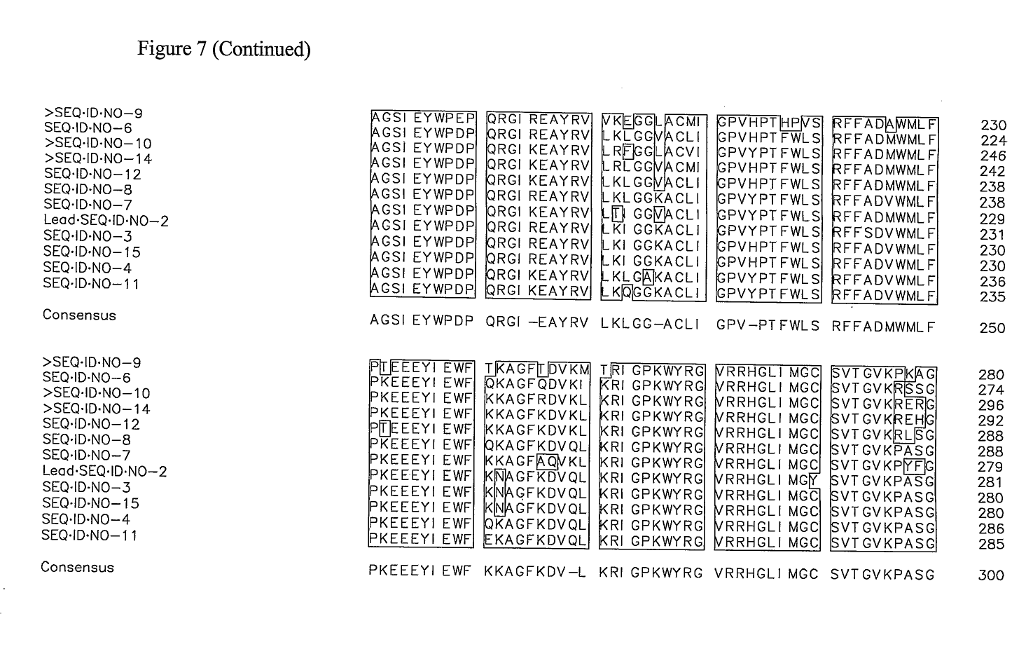

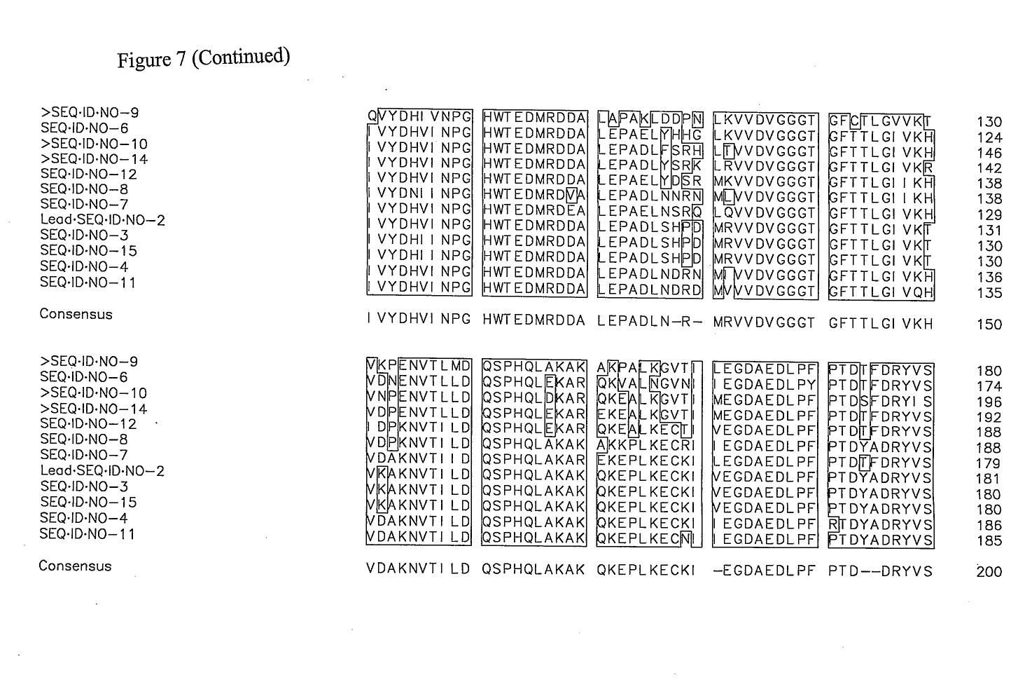

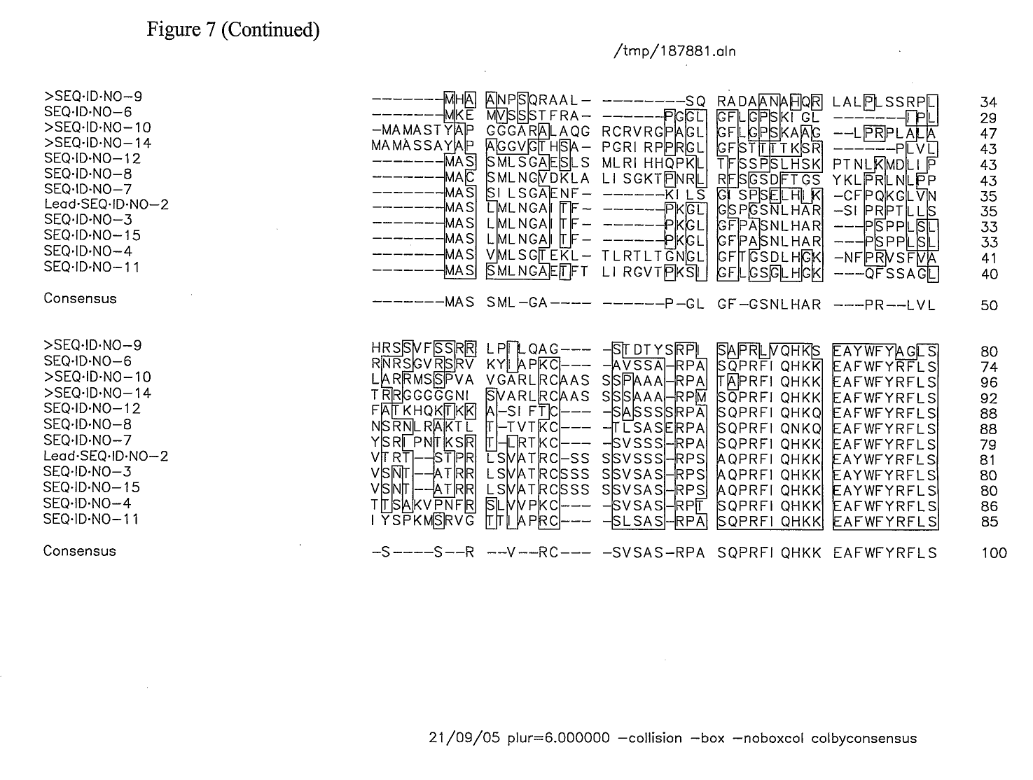

In some cases, a tocopherol-modulating polypeptide is a methyltransferase, such as a 2-methyl-6-phytylbenzoquinol (MPBQ)/2-methyl-6-solanylbenzoquinol (MSBQ) methyltransferase (Cheng et al., Plant Cell 15:2343-56 (2003)). Methyltransferases are involved in the metabolism of, inter alia, various vitamins in plants. For example, key reactions in biosynthetic pathways to tocopherols, ubiquinones, and other nutritionally valuable phytonutrients often involve methyltransferases. A methyltransferase polypeptide, such as a MPBQ/MSBQ methyltransferase polypeptide, can have a Ubie_methyltran domain characteristic of polypeptides belonging to the ubiE/COQ5 methyltransferase family of polypeptides. Members of this polypeptide family include ubiquinone/menaquinone biosynthesis methyltransferases such as the C-methyltransferase from the ubiE gene of Escherichia coli, ubiquinone biosynthesis methyltransferases such as the C-methyltransferase from the COQ5 gene of Saccharomyces cerevisiae, menaquinone biosynthesis methyltransferases such as the C-methyltransferase from the MENH gene of Bacillus subtilis, as well as methyltransferases involved in biotin and sterol biosynthesis and in phosphatidylethanolamine methylation. SEQ ID NO:2 shown in FIG. 2 sets forth the amino acid sequence of an Arabidopsis MPBQ/MSBQ methyltransferase clone identified herein as Ceres clone 19143, that is predicted to contain a Ubie_methyltran domain. Amino acid sequences of orthologs of the polypeptide having the amino acid sequence set forth in SEQ ID NO:2 are provided in FIG. 7.

A tocopherol-modulating polypeptide can be a polypeptide including the amino acid sequence set forth in SEQ ID NO:2. Alternatively, a tocopherol-modulating polypeptide can be a homolog, ortholog, or variant of the polypeptide having the amino acid sequence set forth in SEQ ID NO:2. For example, a tocopherol-modulating polypeptide can have an amino acid sequence with at least 60 percent sequence identity (e.g., 61 percent, 66 percent, 67 percent, 70 percent, 72 percent, 74 percent, 76 percent, 77 percent, 78 percent, 79 percent, 80 percent, 81 percent, 82 percent, 84 percent, 85 percent, 87 percent, 90 percent, 92 percent, 94 percent, 95 percent, 96 percent, 97 percent, 98 percent, or 99 percent sequence identity) to the amino acid sequence set forth in SEQ ID NO:2.

For example, a tocopherol-modulating polypeptide can include a polypeptide corresponding to Ceres clone 1061027 (SEQ ID NO:3), Ceres clone 480158 (SEQ ID NO:4), Ceres clone 656984 (SEQ ID NO:5), gi|50934645 (SEQ ID NO:6), gi|1419090 (SEQ ID NO:7), gi|21228 (SEQ ID NO:8), gi/37265798 (SEQ ID NO:9), SEQ ID NO:22 set forth in U.S. Patent Application No. 20030150015 (SEQ ID NO:10), SEQ ID NO:23 set forth in U.S. Patent Application No. 20030150015 (SEQ ID NO:11), SEQ ID NO:24 set forth in U.S. Patent Application No. 20030150015 (SEQ ID NO:12), SEQ ID NO:25 set forth in U.S. Patent Application No. 20030150015 (SEQ ID NO:13), SEQ ID NO:26 set forth in U.S. Patent Application No. 20030150015 (SEQ ID NO:14), SEQ ID NO:27 set forth in U.S. Patent Application No. 20030150015 (SEQ ID NO:15), Ceres CLONE ID no. 183492 (SEQ ID NO:17), Ceres CLONE ID no. 1925254 (SEQ ID NO:19), Ceres CLONE ID no. 1792831 (SEQ ID NO:21), Ceres CLONE ID no. 1804277 (SEQ ID NO:23), or the consensus sequence set forth in FIG. 7.

In some cases, a tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:17, SEQ ID NO:19, SEQ ID NO:21, SEQ ID NO:23, or the consensus sequence set forth in FIG. 7.

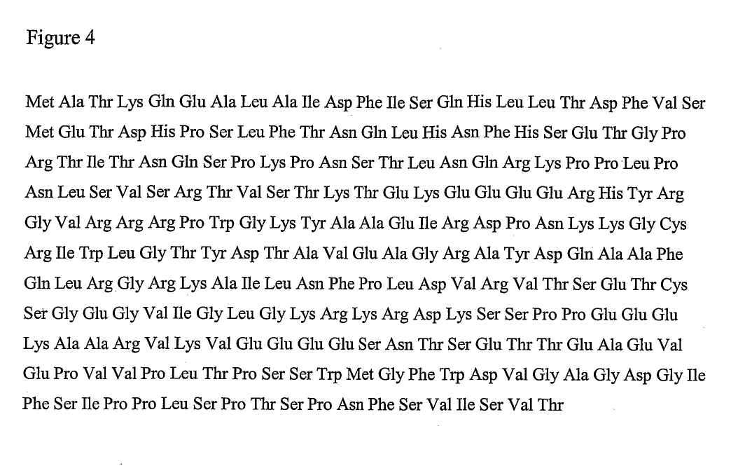

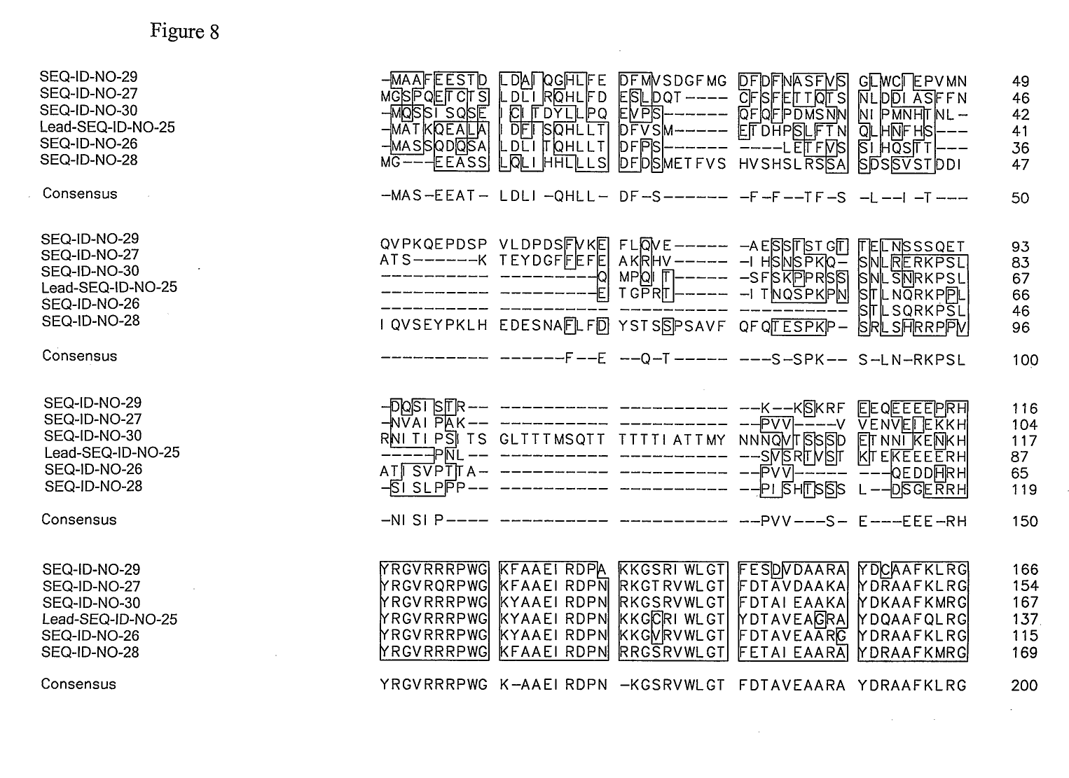

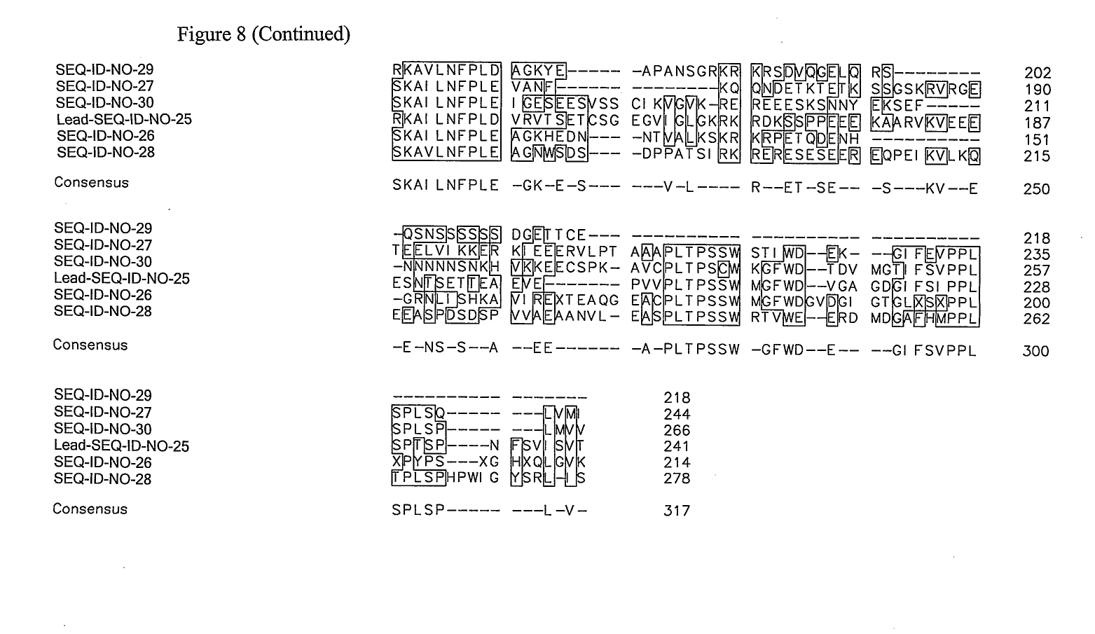

In other cases, a tocopherol-modulating polypeptide is a transcription factor, such as a DNA binding protein-like protein. A DNA binding protein-like protein is a polypeptide that is similar to a DNA-binding protein. A transcription factor polypeptide, such as a DNA binding protein-like protein, can have an AP2 domain characteristic of polypeptides belonging to the AP2/EREBP family of plant transcription factor polypeptides. AP2 (APETALA2) and EREBPs (ethylene-responsive element binding proteins) are prototypic members of a family of transcription factors unique to plants, whose distinguishing characteristic is that they contain the so-called AP2 DNA binding domain. AP2/EREBP genes form a large multigene family encoding polypeptides that play a variety of roles throughout the plant life cycle: from being key regulators of several developmental processes, such as floral organ identity determination and control of leaf epidermal cell identity, to forming part of the mechanisms used by plants to respond to various types of biotic and environmental stress. SEQ ID NO:25 shown in FIG. 4 sets forth the amino acid sequence of an Arabidopsis clone, identified herein as Ceres clone 92102, that is predicted to encode a DNA binding protein-like protein containing an AP2 domain. Orthologs of the polypeptide having the amino acid sequence set forth in SEQ ID NO:25 are provided in FIG. 8.

A tocopherol-modulating polypeptide can be a polypeptide including the amino acid sequence set forth in SEQ ID NO:25. Alternatively, a tocopherol-modulating polypeptide can be a homolog, ortholog, or variant of the polypeptide having the amino acid sequence set forth in SEQ ID NO:25. For example, a tocopherol-modulating polypeptide can have an amino acid sequence with at least 40 percent sequence identity (e.g., 41 percent, 43 percent, 46 percent, 48 percent, 50 percent, 54 percent, 58 percent, 59 percent, 61 percent, 66 percent, 67 percent, 70 percent, 72 percent, 74 percent, 76 percent, 77 percent, 78 percent, 79 percent, 80 percent, 81 percent, 82 percent, 84 percent, 85 percent, 87 percent, 90 percent, 92 percent, 94 percent, 95 percent, 96 percent, 97 percent, 98 percent, or 99 percent sequence identity) to the amino acid sequence set forth in SEQ ID NO:25.

For example, a tocopherol-modulating polypeptide can include a polypeptide corresponding to Ceres clone 965028 (SEQ ID NO:26), gi|45642990 (SEQ ID NO:27), gi|40060531 (SEQ ID NO:28), gi|38260618 (SEQ ID NO:29), Ceres clone 548557 (SEQ ID NO:30), or the consensus sequence set forth in FIG. 8.

In some cases, a tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:26, SEQ ID NO:27, SEQ ID NO:28, SEQ ID NO:29, SEQ ID NO:30, or the consensus sequence set forth in FIG. 8.

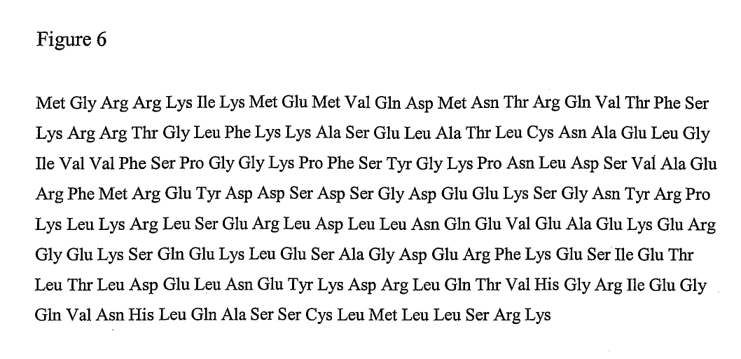

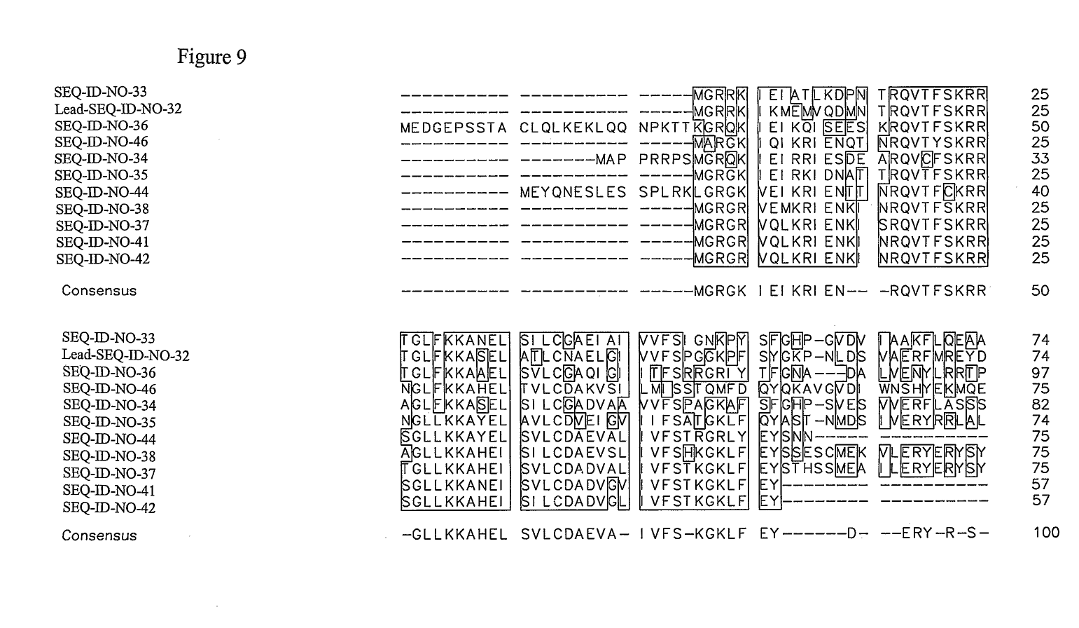

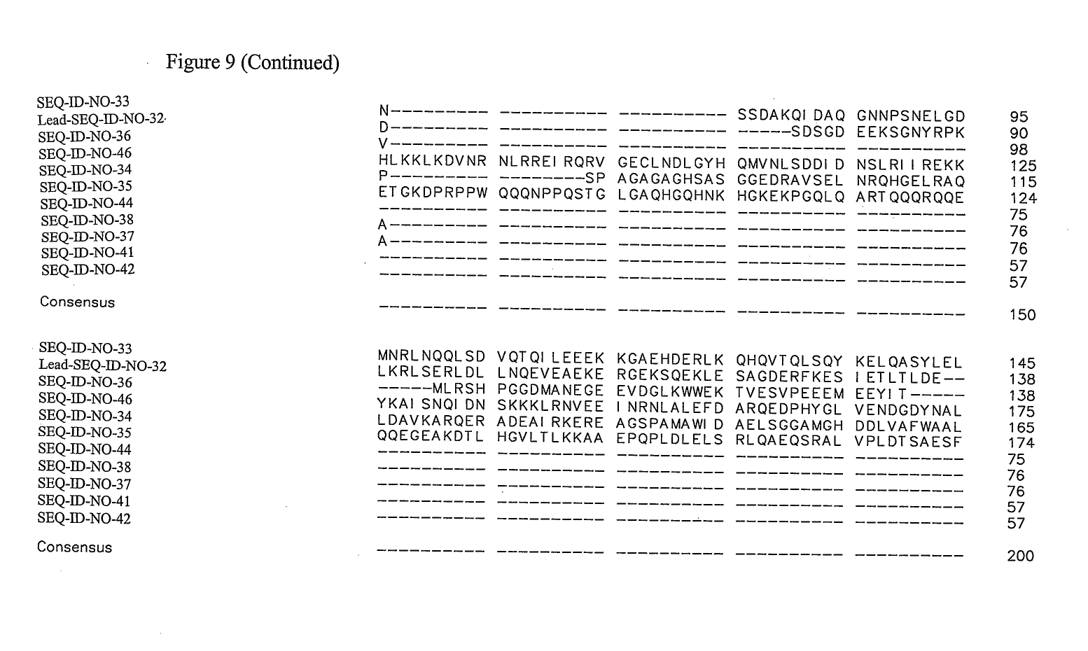

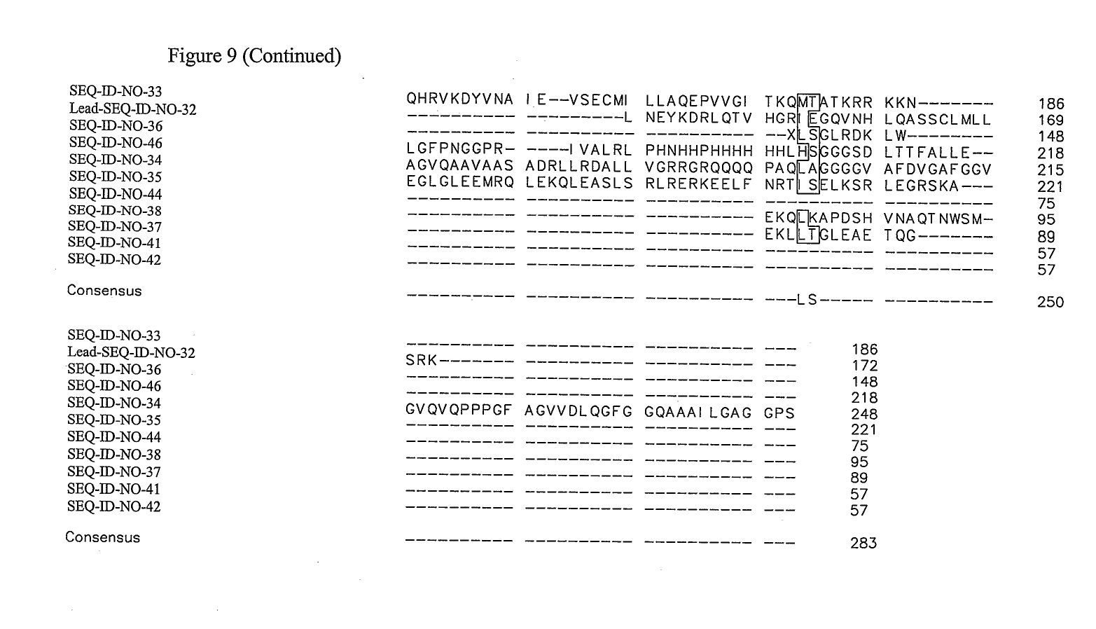

In some cases, a tocopherol-modulating polypeptide is a MADS-box transcription factor. MADS-box transcription factors are key regulators of several plant development processes. The MADS box is a highly conserved sequence motif found in a family of transcription factors. The conserved domain was recognized after the first four members of the family, MCM1, AGAMOUS, DEFICIENS and SRF (serum response factor), were identified. Polypeptides belonging to the MADS family function as dimers, the primary DNA-binding element of which is an anti-parallel coiled coil of two amphipathic alpha-helices, one from each subunit. The DNA wraps around the coiled coil allowing the basic N-termini of the helices to fit into the DNA major groove. The chain extending from the helix N-termini reaches over the DNA backbone and penetrates into the minor groove. A four-stranded, anti-parallel beta-sheet packs against the coiled-coil face opposite the DNA and is the central element of the dimerization interface. SEQ ID NO:32 shown in FIG. 6 sets forth the amino acid sequence encoded by a nucleic acid sequence from Arabidopsis, identified herein as Ceres cDNA 23495742, that is predicted to encode a MADS-box transcription factor. Orthologs of the polypeptide having the amino acid sequence set forth in SEQ ID NO:32 are provided in FIG. 9.

A tocopherol-modulating polypeptide can be a polypeptide including the amino acid sequence set forth in SEQ ID NO:32. Alternatively, a tocopherol-modulating polypeptide can be a homolog, ortholog, or variant of the polypeptide having the amino acid sequence set forth in SEQ ID NO:32. For example, a tocopherol-modulating polypeptide can have an amino acid sequence with at least 40 percent sequence identity (e.g., 41 percent, 43 percent, 46 percent, 48 percent, 50 percent, 54 percent, 58 percent, 59 percent, 61 percent, 66 percent, 67 percent, 70 percent, 72 percent, 74 percent, 76 percent, 77 percent, 78 percent, 79 percent, 80 percent, 81 percent, 82 percent, 84 percent, 85 percent, 87 percent, 90 percent, 92 percent, 94 percent, 95 percent, 96 percent, 97 percent, 98 percent, or 99 percent sequence identity) to the amino acid sequence set forth in SEQ ID NO:32.

For example, a tocopherol-modulating polypeptide can include a polypeptide corresponding to Ceres clone 681294 (SEQ ID NO:33), Ceres clone 244495 (SEQ ID NO:34), gi|57999638 (SEQ ID NO:35), Ceres clone 1067477 (SEQ ID NO: 36), Ceres clone 1604678 (SEQ ID NO:37), gi|45533872 (SEQ ID NO:38), gi|45533888 (SEQ ID NO:39), gi|45533884 (SEQ ID NO:40), gi|27372827 (SEQ ID NO:41), gi|27372831 (SEQ ID NO:42), gi|27372829 (SEQ ID NO:43), gi|34922009 (SEQ ID NO:44), gi|34922000 (SEQ ID NO:45), gi|42795299 (SEQ ID NO:46), or the consensus sequence set forth in FIG. 9.

In some cases, a tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, or the consensus sequence set forth in FIG. 9.

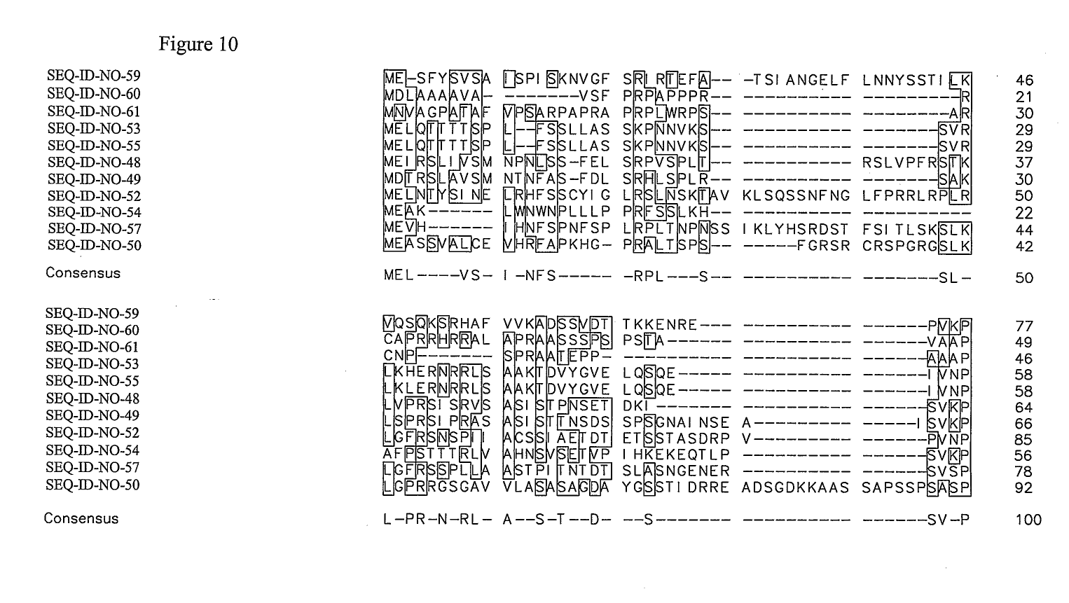

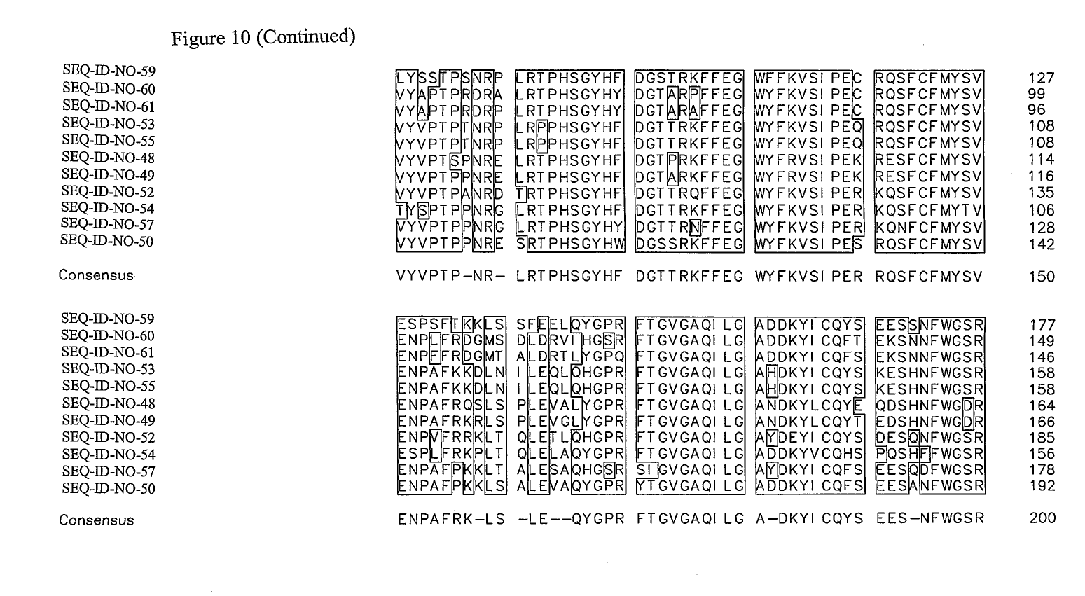

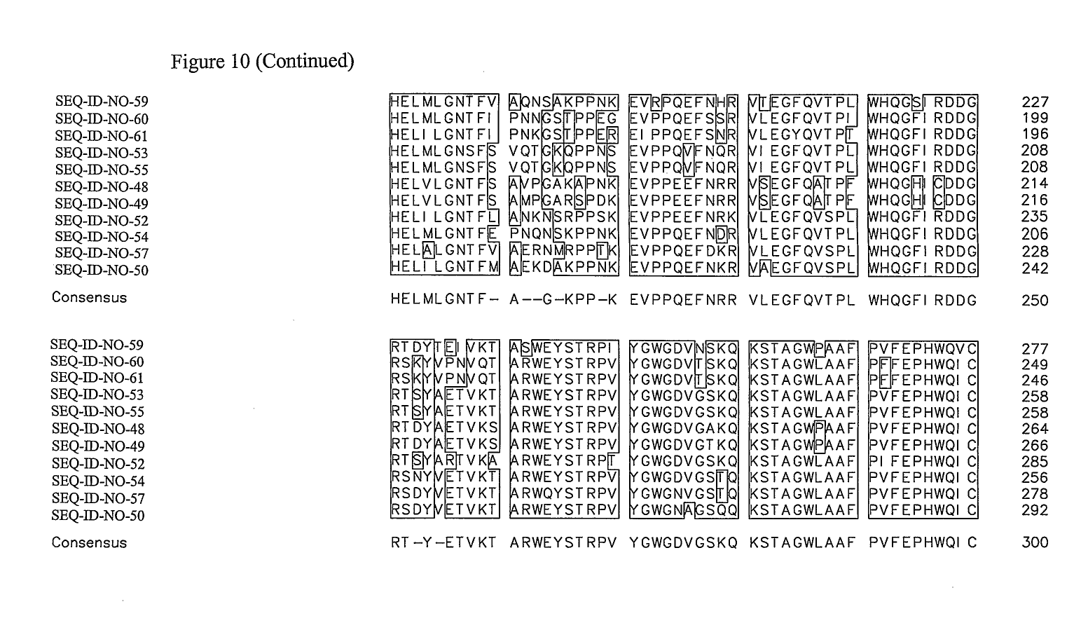

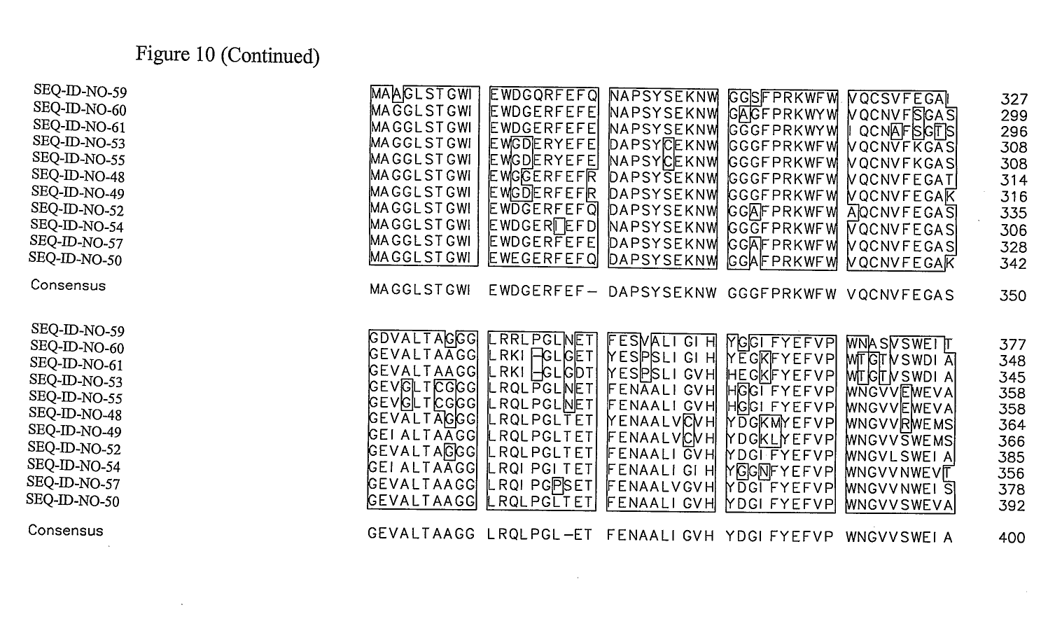

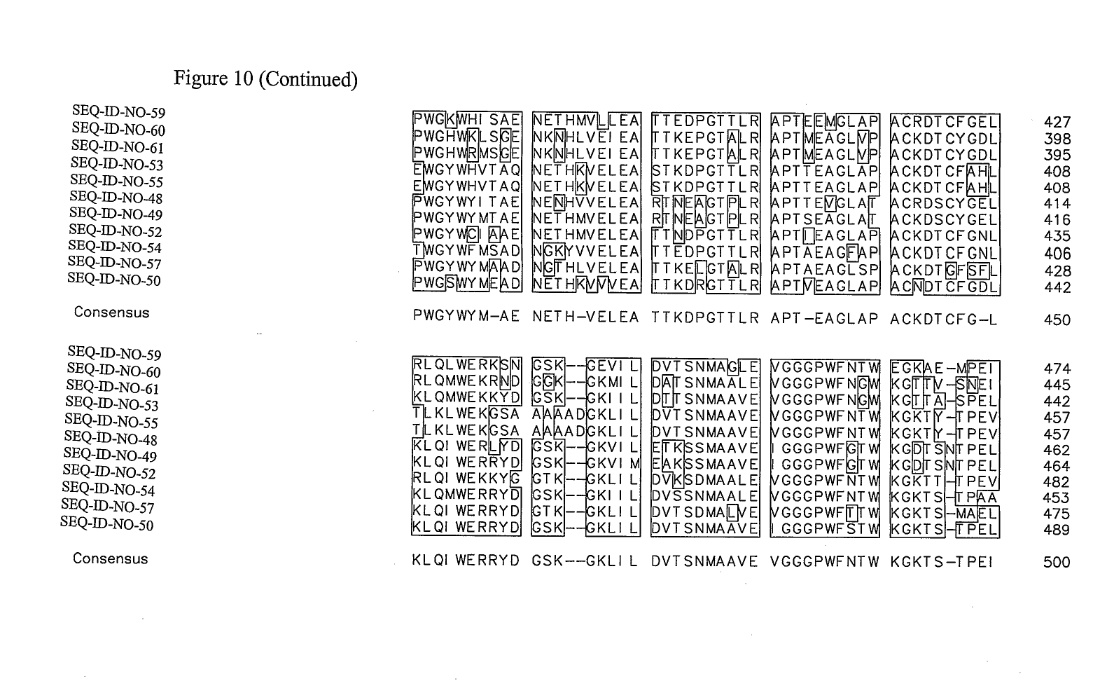

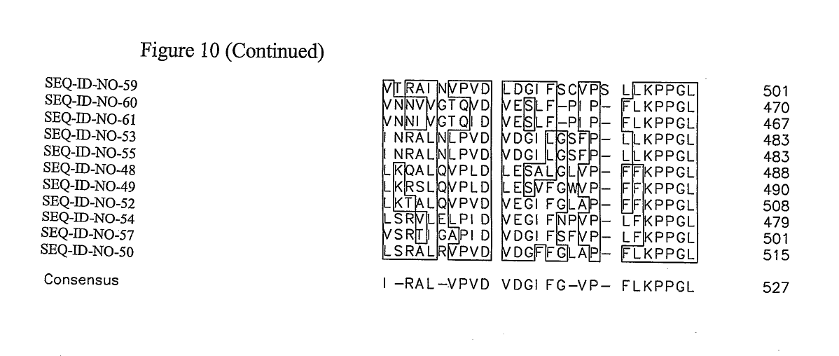

In some cases, a tocopherol-modulating polypeptide is a tocopherol cyclase 1 polypeptide. Tocopherol cyclase polypeptides catalyze the reaction: alkene group+alcohol group on same molecule=cyclic ether. Substrates include 2-methyl-6-phytyl-1,4-hydroquinone and 2,3-dimethyl-5-phytyl-1,4-hydroquinone. SEQ ID NO:48 sets forth the amino acid sequence encoded by a nucleic acid sequence from Arabidopsis, identified herein as Ceres ANNOT ID 567302, that is predicted to encode a tocopherol cyclase polypeptide. Orthologs of the polypeptide having the amino acid sequence set forth in SEQ ID NO:48 are provided in FIG. 10.

A tocopherol-modulating polypeptide can be a polypeptide including the amino acid sequence set forth in SEQ ID NO:48. Alternatively, a tocopherol-modulating polypeptide can be a homolog, ortholog, or variant of the polypeptide having the amino acid sequence set forth in SEQ ID NO:48. For example, a tocopherol-modulating polypeptide can have an amino acid sequence with at least 55 percent sequence identity (e.g., 56 percent, 58 percent, 59 percent, 61 percent, 66 percent, 67 percent, 70 percent, 72 percent, 74 percent, 76 percent, 77 percent, 78 percent, 79 percent, 80 percent, 81 percent, 82 percent, 84 percent, 85 percent, 87 percent, 90 percent, 92 percent, 94 percent, 95 percent, 96 percent, 97 percent, 98 percent, or 99 percent sequence identity) to the amino acid sequence set forth in SEQ ID NO:48.

For example, a tocopherol-modulating polypeptide can include a polypeptide corresponding to Ceres CLONE ID no. 1109488 (SEQ ID NO:49), Public GI no. 33188419 (SEQ ID NO:50), Ceres CLONE ID no. 1948913 (SEQ ID NO:52), Public GI no. 80971684 (SEQ ID NO:53), Ceres CLONE ID no. 1245537 (SEQ ID NO:54), Public GI no. 80971690 (SEQ ID NO:55), Ceres ANNOT ID no. 1530974 (SEQ ID NO:57), Ceres CLONE ID no. 574132 (SEQ ID NO:58), Public GI no. 47078321 (SEQ ID NO:59), Public GI no. 50906901 (SEQ ID NO:60), Ceres CLONE ID no. 754013 (SEQ ID NO:61), Public GI no. 91694297 (SEQ ID NO:62), or the consensus sequence set forth in FIG. 10.

In some cases, a tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:49, SEQ ID NO:50, SEQ ID NO:52, SEQ ID NO:53, SEQ ID NO:54, SEQ ID NO:55, SEQ ID NO:57, SEQ ID NO:58, SEQ ID NO:59, SEQ ID NO:60, SEQ ID NO:61, SEQ ID NO:62, or the consensus sequence set forth in FIG. 10.

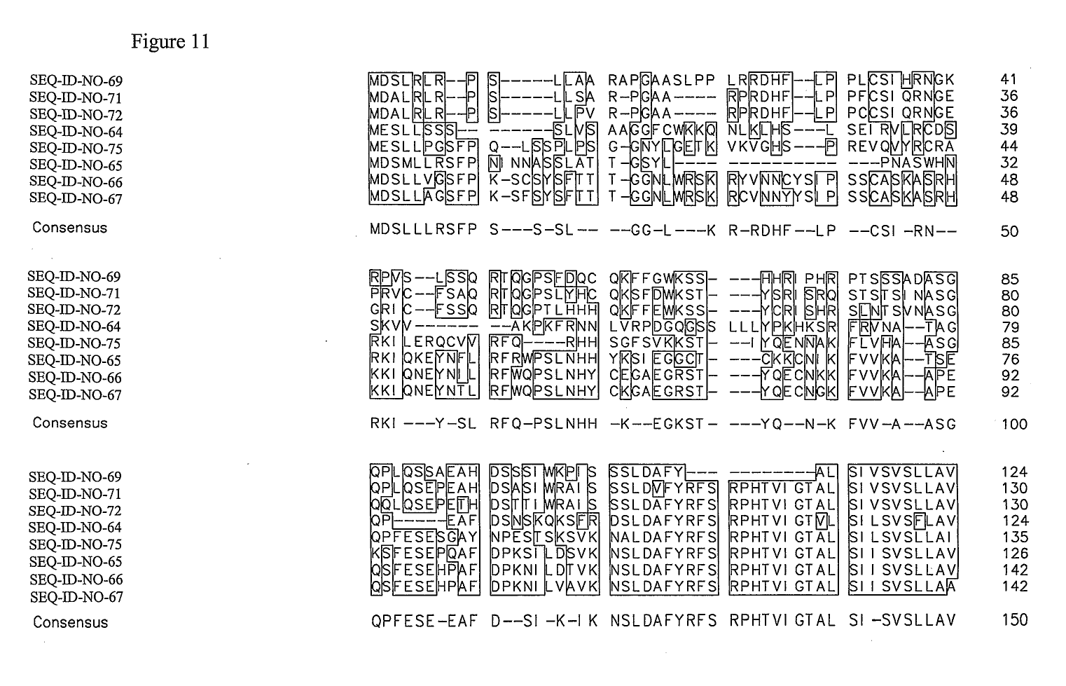

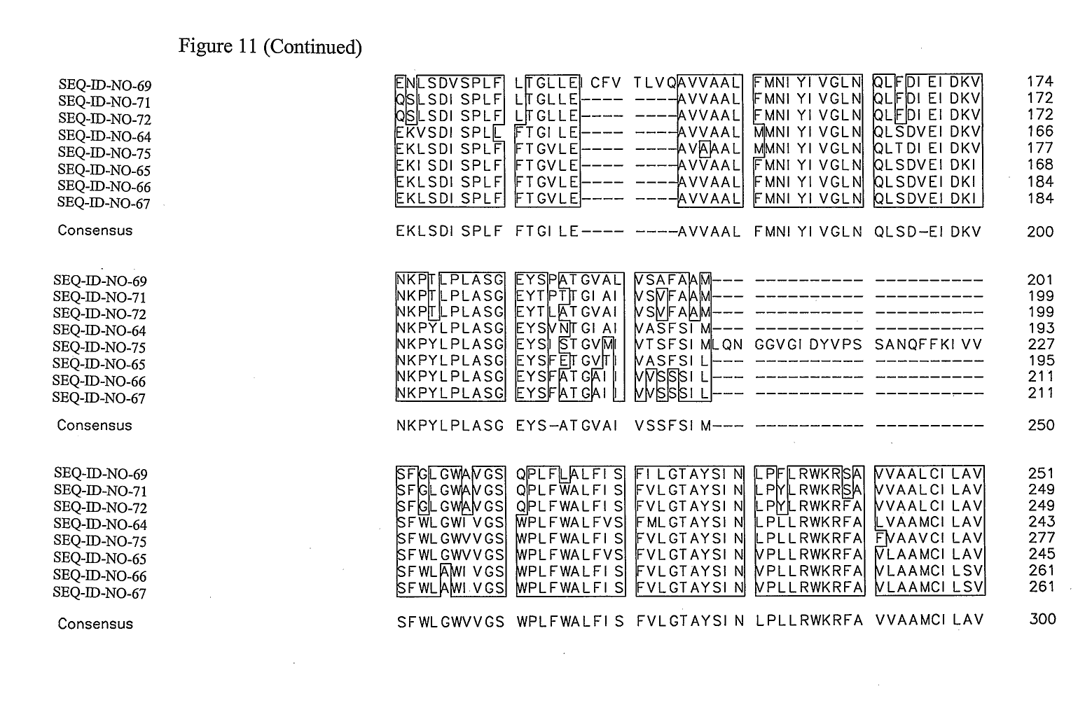

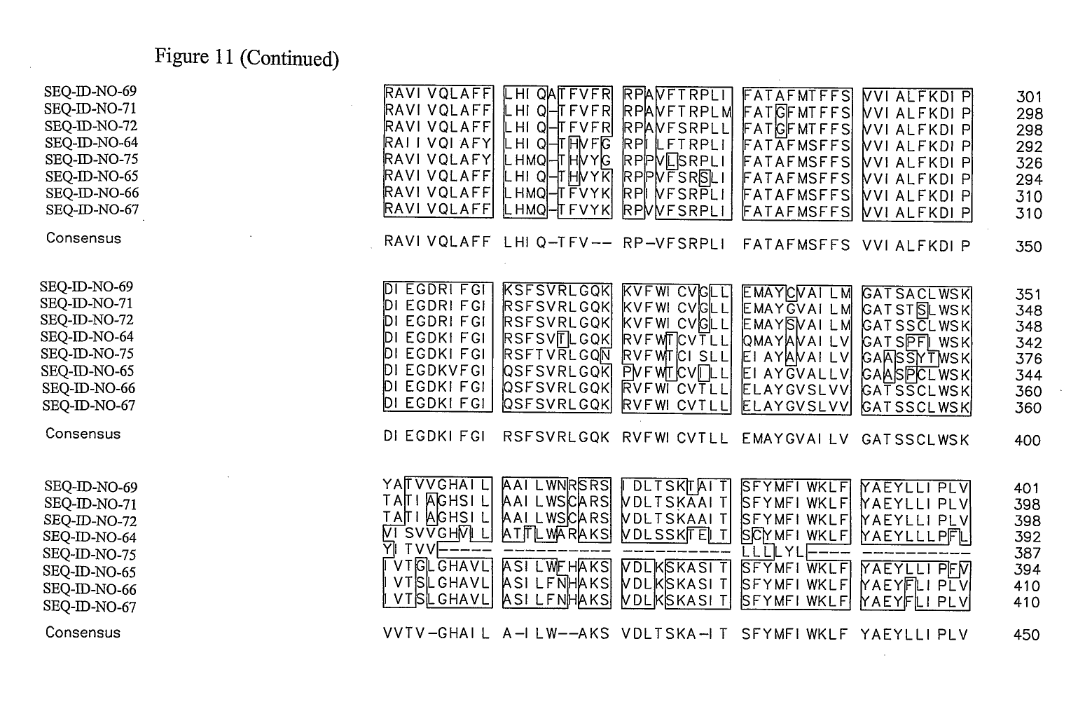

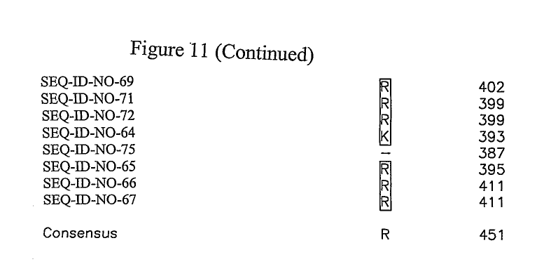

In some cases, a tocopherol-modulating polypeptide is a homogentisate phytylprenyltransferase polypeptide. Homogentisate phytylprenyltransferase polypeptides catalyze the reaction: homogentisic acid+phytyl diphosphate=2-methyl-6-phytyl-1,4-benzoquinone. SEQ ID NO:64 sets forth the amino acid sequence encoded by a nucleic acid sequence from Arabidopsis, identified herein as Ceres ANNOT ID 552252, that is predicted to encode a homogentisate phytylprenyltransferase polypeptide. Orthologs of the polypeptide having the amino acid sequence set forth in SEQ ID NO:64 are provided in FIG. 11.

A tocopherol-modulating polypeptide can be a polypeptide including the amino acid sequence set forth in SEQ ID NO:64. Alternatively, a tocopherol-modulating polypeptide can be a homolog, ortholog, or variant of the polypeptide having the amino acid sequence set forth in SEQ ID NO:64. For example, a tocopherol-modulating polypeptide can have an amino acid sequence with at least 60 percent sequence identity (e.g., 61 percent, 66 percent, 67 percent, 70 percent, 72 percent, 74 percent, 76 percent, 77 percent, 78 percent, 79 percent, 80 percent, 81 percent, 82 percent, 84 percent, 85 percent, 87 percent, 90 percent, 92 percent, 94 percent, 95 percent, 96 percent, 97 percent, 98 percent, or 99 percent sequence identity) to the amino acid sequence set forth in SEQ ID NO:64.

For example, a tocopherol-modulating polypeptide can include a polypeptide corresponding to Public GI no. 81295666 (SEQ ID NO:65), Public GI no. 51949754 (SEQ ID NO:66), Public GI no. 92882118 (SEQ ID NO:67), Public GI no. 61808320 (SEQ ID NO:68), Public GI no. 51536170 (SEQ ID NO:69), Ceres CLONE ID no. 1789748 (SEQ ID NO:71), Ceres CLONE ID no. 395119 (SEQ ID NO:72), Public GI no. 81295658 (SEQ ID NO:73), Ceres ANNOT ID no. 1478147 (SEQ ID NO:75), or the consensus sequence set forth in FIG. 11.

In some cases, a tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:65, SEQ ID NO:66, SEQ ID NO:67, SEQ ID NO:68, SEQ ID NO:69, SEQ ID NO:71, SEQ ID NO:72, SEQ ID NO:73, SEQ ID NO:75, or the consensus sequence set forth in FIG. 11.

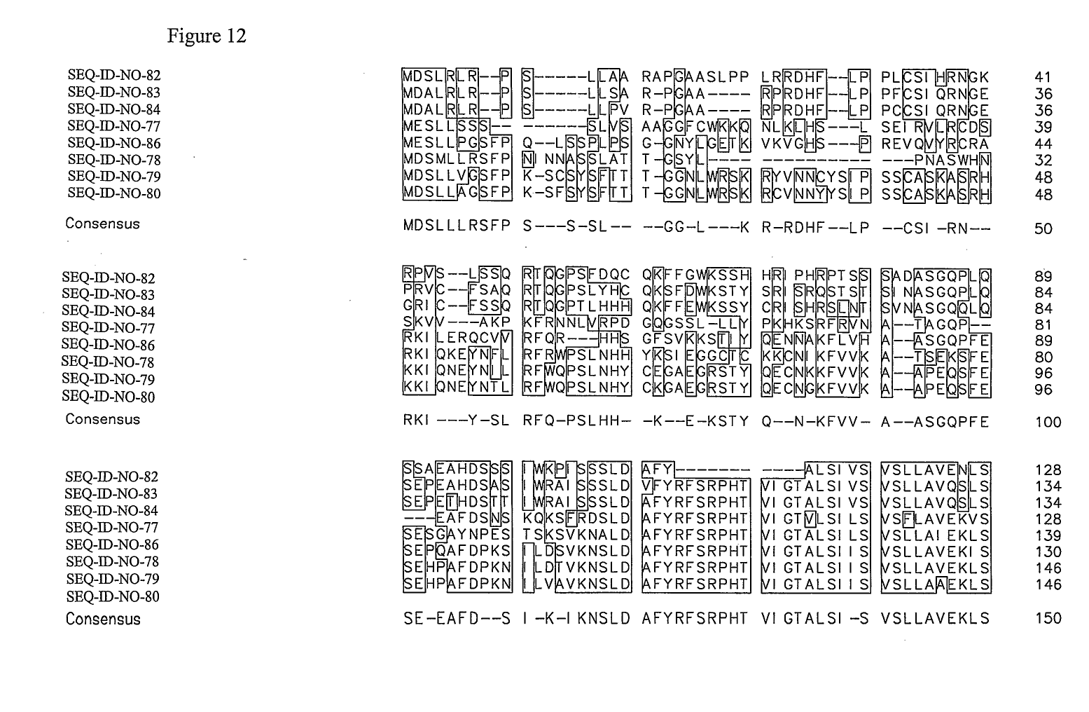

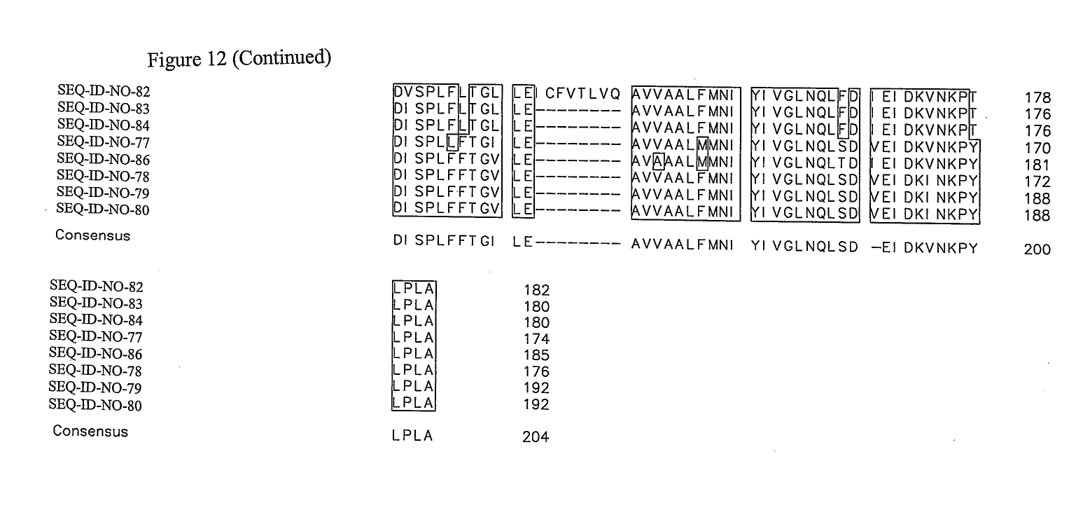

In some cases, a tocopherol-modulating polypeptide is a polypeptide that does not have homology to an existing polypeptide family based on Pfam analysis. SEQ ID NO:77 sets forth the amino acid sequence encoded by a nucleic acid sequence from Arabidopsis, identified herein as Ceres ANNOT ID no. 859061, that is predicted to encode a polypeptide that does not have homology to an existing polypeptide family based on Pfam analysis. Orthologs of the polypeptide having the amino acid sequence set forth in SEQ ID NO:77 are provided in FIG. 12.

A tocopherol-modulating polypeptide can be a polypeptide including the amino acid sequence set forth in SEQ ID NO:77. Alternatively, a tocopherol-modulating polypeptide can be a homolog, ortholog, or variant of the polypeptide having the amino acid sequence set forth in SEQ ID NO:77. For example, a tocopherol-modulating polypeptide can have an amino acid sequence with at least 45 percent sequence identity (e.g., 50 percent, 55 percent, 61 percent, 66 percent, 67 percent, 70 percent, 72 percent, 74 percent, 76 percent, 77 percent, 78 percent, 79 percent, 80 percent, 81 percent, 82 percent, 84 percent, 85 percent, 87 percent, 90 percent, 92 percent, 94 percent, 95 percent, 96 percent, 97 percent, 98 percent, or 99 percent sequence identity) to the amino acid sequence set forth in SEQ ID NO:77.

For example, a tocopherol-modulating polypeptide can include a polypeptide corresponding to Public GI no. 81295666_T (SEQ ID NO:78), Public GI no. 51949754T (SEQ ID NO:79), Public GI no. 92882118_T (SEQ ID NO:80), Public GI no. 61808320_T (SEQ ID NO:81), Public GI no. 51536170_T (SEQ ID NO:82), Ceres CLONE ID no. 1789748_T (SEQ ID NO:83), Ceres CLONE ID no. 395119_T (SEQ ID NO:84), Public GI no. 81295658_T (SEQ ID NO:85), Ceres ANNOT ID no. 1478147_T (SEQ ID NO:86), or the consensus sequence set forth in FIG. 12.

In some cases, a tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, or the consensus sequence set forth in FIG. 12.

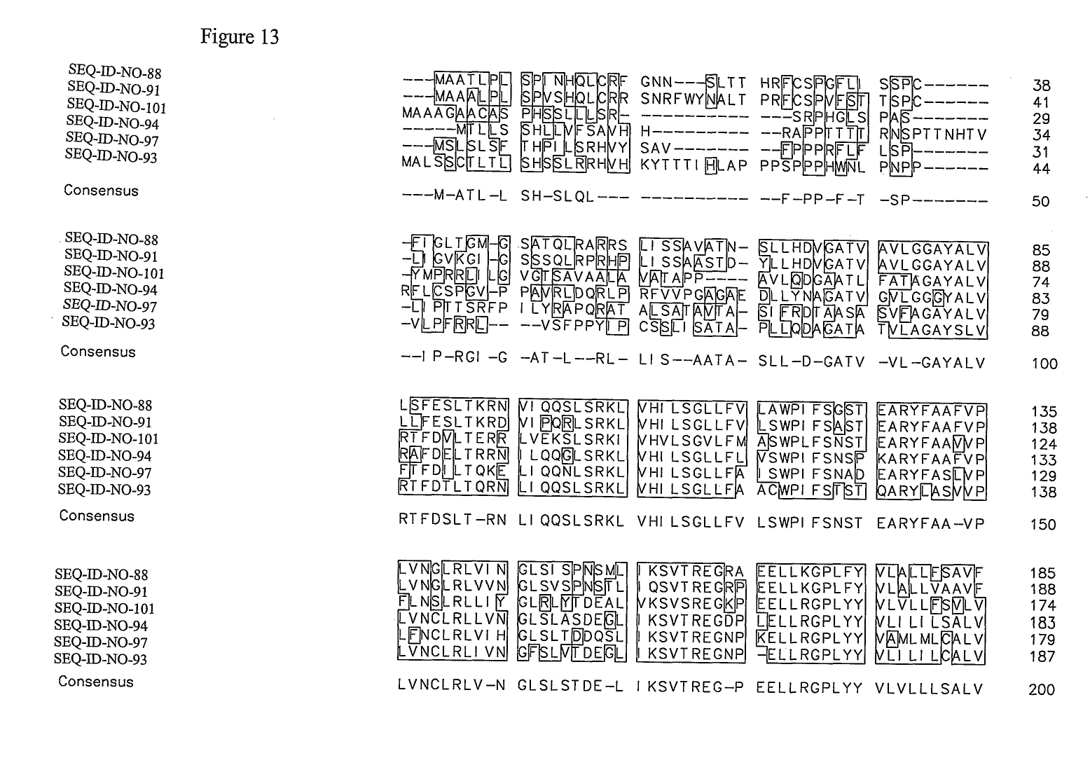

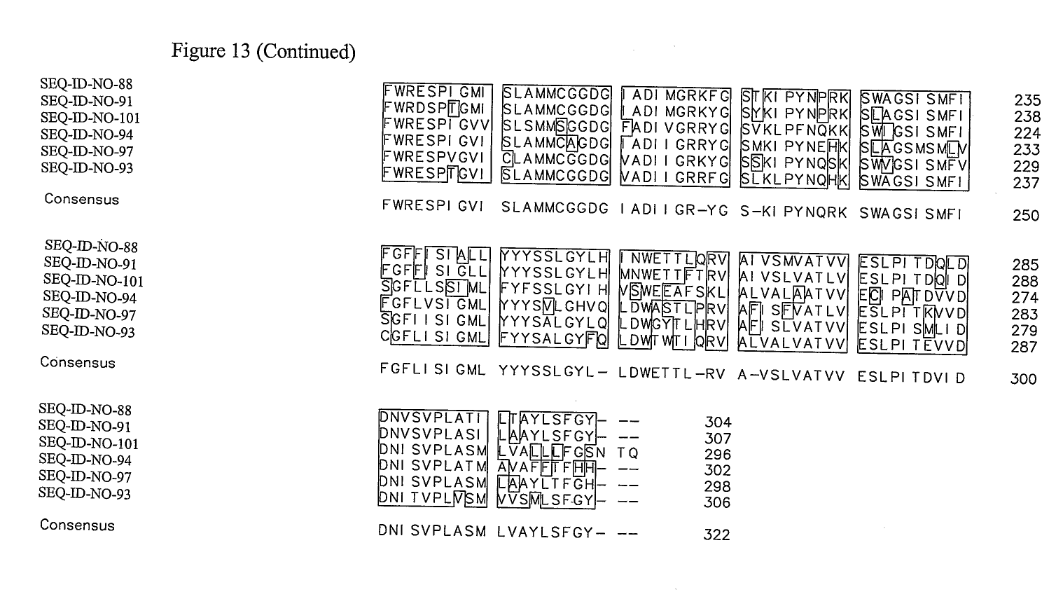

In some cases, a tocopherol-modulating polypeptide has a CTP_transf_{—1} domain characteristic of polypeptides belonging to the cytidylyltransferase polypeptide family. Members of this family are integral membrane polypeptide cytidylyltransferases. One member of this family, phosphatidate cytidylyltransferase (also known as CDP-diacylglycerol synthase or CDS), catalyzes the synthesis of CDP-diacylglycerol from CTP and phosphatidate. CDP-diacylglycerol is an important branch point intermediate in both prokaryotic and eukaryotic organisms. SEQ ID NO:88 sets forth the amino acid sequence encoded by a nucleic acid sequence from Arabidopsis, identified herein as Ceres CLONE ID no. 125255, that is predicted to encode a polypeptide having a CTP_transf_{—}1 domain. Orthologs of the polypeptide having the amino acid sequence set forth in SEQ ID NO:88 are provided in FIG. 13.

A tocopherol-modulating polypeptide can be a polypeptide including the amino acid sequence set forth in SEQ ID NO:88. Alternatively, a tocopherol-modulating polypeptide can be a homolog, ortholog, or variant of the polypeptide having the amino acid sequence set forth in SEQ ID NO:88. For example, a tocopherol-modulating polypeptide can have an amino acid sequence with at least 50 percent sequence identity (e.g., 52 percent, 55 percent, 61 percent, 66 percent, 67 percent, 70 percent, 72 percent, 74 percent, 76 percent, 77 percent, 78 percent, 79 percent, 80 percent, 81 percent, 82 percent, 84 percent, 85 percent, 87 percent, 90 percent, 92 percent, 94 percent, 95 percent, 96 percent, 97 percent, 98 percent, or 99 percent sequence identity) to the amino acid sequence set forth in SEQ ID NO:88.

For example, a tocopherol-modulating polypeptide can include a polypeptide corresponding to Public GI no. 7406453 (SEQ ID NO:89), Public GI no. 28393229 (SEQ ID NO:90), Ceres CLONE ID no. 1377623 (SEQ ID NO:91), Ceres ANNOT ID no. 1518536 (SEQ ID NO:93), Public GI no. 76443937 (SEQ ID NO:94), Ceres CLONE ID no. 464672 (SEQ ID NO:95), Ceres CLONE ID no. 1940214 (SEQ ID NO:97), Public GI no. 76443931 (SEQ ID NO:98), Ceres CLONE ID no. 287069 (SEQ ID NO:99), Ceres CLONE ID no. 1780314 (SEQ ID NO:101), Public GI no. 76443929 (SEQ ID NO:102), or the consensus sequence set forth in FIG. 13.

In some cases, a tocopherol-modulating polypeptide can include a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:88, SEQ ID NO:89, SEQ ID NO:90, SEQ ID NO:91, SEQ ID NO:93, SEQ ID NO:94, SEQ ID NO:95, SEQ ID NO:97, SEQ ID NO:98, SEQ ID NO:99, SEQ ID NO:101, SEQ ID NO:102, or the consensus sequence set forth in FIG. 13.

A consensus amino acid sequence for a tocopherol-modulating polypeptide can be determined by aligning amino acid sequences from a variety of plant species and determining the most common amino acid or type of amino acid at each position. For example, a consensus sequence can be determined by aligning amino acid sequences corresponding to SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:14, and SEQ ID NO:15 as shown in FIG. 7. In another embodiment, a consensus sequence can be determined by aligning amino acid sequences corresponding to SEQ ID NO:25, SEQ ID NO:26, SEQ ID NO:27, SEQ ID NO:28, SEQ ID NO:29 and SEQ ID NO:30 as shown in FIG. 8. In another embodiment, a consensus sequence can be determined by aligning amino acid sequences corresponding to SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:44, and SEQ ID NO:46 as shown in FIG. 9. In another embodiment, a consensus sequence can be determined by aligning amino acid sequences corresponding to SEQ ID NO:48, SEQ ID NO:49, SEQ ID NO:50, SEQ ID NO:52, SEQ ID NO:53, SEQ ID NO:54, SEQ ID NO:55, SEQ ID NO:57, SEQ ID NO:59, SEQ ID NO:60, and SEQ ID NO:61 as shown in FIG. 10. In another embodiment, a consensus sequence can be determined by aligning amino acid sequences corresponding to SEQ ID NO:64, SEQ ID NO:65, SEQ ID NO:66, SEQ ID NO:67, SEQ ID NO:69, SEQ ID NO:71, SEQ ID NO:72, and SEQ ID NO:75 as shown in FIG. 11. In another embodiment, a consensus sequence can be determined by aligning amino acid sequences corresponding to SEQ ID NO:77, SEQ ID NO:78, SEQ ID NO:79, SEQ ID NO:80, SEQ ID NO:82, SEQ ID NO:83, SEQ ID NO:84, and SEQ ID NO:86 as shown in FIG. 12. In another embodiment, a consensus sequence can be determined by aligning amino acid sequences corresponding to SEQ ID NO:88, SEQ ID NO:91, SEQ ID NO:93, SEQ ID NO:94, SEQ ID NO:97, and SEQ ID NO:101 as shown in FIG. 13.

A dash in an aligned sequence in FIGS. 7-13 represents a gap, i.e., a lack of an amino acid at that position. Identical amino acids or conserved amino acid substitutions among aligned sequences are identified by boxes.

Each consensus sequence is comprised of conserved regions. Each conserved region contains a sequence of contiguous amino acid residues. A dash in a consensus sequence indicates that the consensus sequence either lacks an amino acid at that position or includes an amino acid at that position. If an amino acid is present, the residue at that position corresponds to one found in any aligned sequence at that position.

Useful polypeptides can be constructed based on the consensus sequence in any of FIGS. 7-13. Such a polypeptide includes the conserved regions in the selected consensus sequence, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Other means by which tocopherol-modulating polypeptides can be identified include functional complementation of tocopherol-modulating polypeptide mutants. Suitable tocopherol-modulating polypeptides also can be identified by analysis of nucleotide and polypeptide sequence alignments. For example, performing a query on a database of nucleotide or polypeptide sequences can identify orthologs of the polypeptide having the amino acid sequence set forth in SEQ ID NO:2, SEQ ID NO:25, SEQ ID NO:32, SEQ ID NO:48, SEQ ID NO:64, SEQ ID NO:77, or SEQ ID NO:88. Sequence analysis can involve BLAST, Reciprocal BLAST, or PSI-BLAST analysis of nonredundant databases. Those proteins in the database that have greater than 35% sequence identity to the specific query polypeptide can be candidates for further evaluation for suitability as tocopherol-modulating polypeptides. If desired, manual inspection of such candidates can be carried out in order to reduce the number of candidates to be further evaluated. Manual inspection can be performed by selecting those candidates that appear to have domains suspected of being present in tocopherol-modulating polypeptides.

Typically, conserved regions of tocopherol-modulating polypeptides exhibit at least 40% amino acid sequence identity (e.g., at least 45%, at least 50%, at least 60%, at least 70%, at least 80%, or at least 90% amino acid sequence identity). Conserved regions of target and template polypeptides can exhibit at least 92%, 94%, 96%, 98%, or 99% amino acid sequence identity. Amino acid sequence identity can be deduced from amino acid or nucleotide sequences. In certain cases, highly conserved domains can be identified within tocopherol-modulating polypeptides. These conserved regions can be useful in identifying functionally similar polypeptides.

Domains are groups of contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, each domain has been associated with either a conserved primary sequence or a sequence motif. Generally these conserved primary sequence motifs have been correlated with specific in vitro and/or in vivo activities. A domain can be any length, including the entirety of the polynucleotide to be transcribed.

The identification of conserved regions in a template, or subject, polypeptide can facilitate production of variants of wild-type tocopherol-modulating polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Pfam/ and online at genome.wustLedu/Pfam/. Descriptions of the information included at the Pfam database are included in Sonnhammer et al., 1998, Nucl. Acids Res. 26:320-322; Sonnhammer et al., 1997, Proteins 28:405-420; and Bateman et al., 1999, Nucl. Acids Res. 27:260-262. From the Pfam database, consensus sequences of protein motifs and domains can be aligned with the template polypeptide sequence to determine conserved region(s).

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

If desired, the classification of a polypeptide as a tocopherol-modulating polypeptide can be determined using techniques known to those having ordinary skill in the art. These techniques can be divided into two general categories: global tocopherol analysis, and type-specific tocopherol analysis. Global tocopherol analysis techniques can include determining the overall level of tocopherols within a cell, group of cells, or tissue (e.g., non-seed tissue vs. seed tissue). Type-specific tocopherol analysis techniques can include measuring the level of a particular type of tocopherol (i.e., α-, β-, δ-, or γ-tocopherol) or tocotrienol (i.e., α-, β-, δ-, or γ-tocotrienol).

A tocopherol-modulating polypeptide can include additional amino acids that are not involved in modulating gene expression, and thus can be longer than would otherwise be the case. For example, a tocopherol-modulating polypeptide can include an amino acid sequence that functions as a reporter. Such a tocopherol-modulating polypeptide can be a fusion protein in which a green fluorescent protein (GFP) polypeptide is fused to, e.g., SEQ ID NO:25, or in which a yellow fluorescent protein (YFP) polypeptide is fused to, e.g., SEQ ID NO:32. In some embodiments, a tocopherol-modulating polypeptide includes a purification tag, a chloroplast transit peptide, a mitochondrial transit peptide, or a leader sequence added to the amino or carboxyl terminus.

### Polynucleotides

Isolated nucleic acids and polypeptides are provided herein. The terms “nucleic acid” and “polynucleotide” are used interchangeably herein, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus (e.g., pararetrovirus, retrovirus, lentivirus, adenovirus, adeno-associated virus, or herpesvirus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made, for example, by chemical synthesis or using PCR. PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

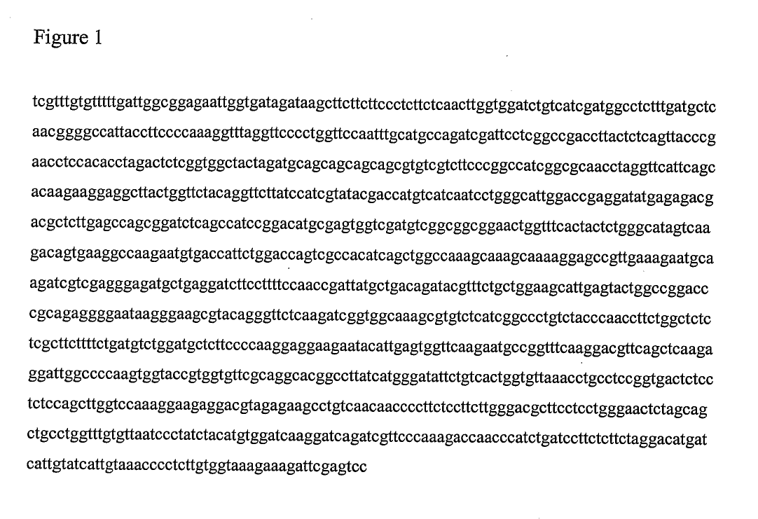

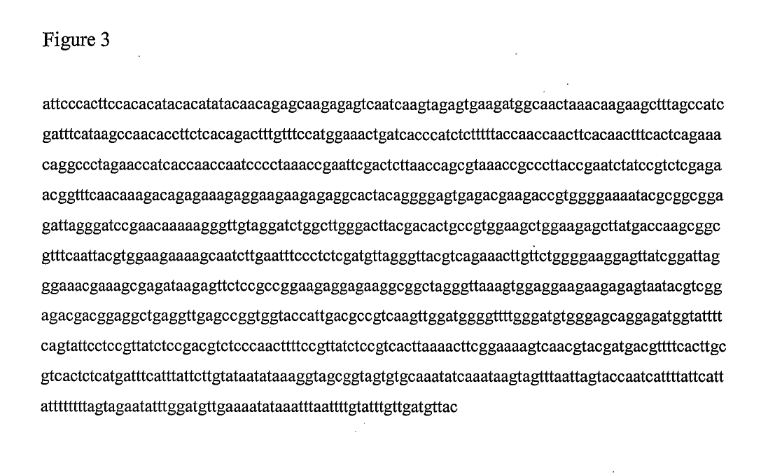

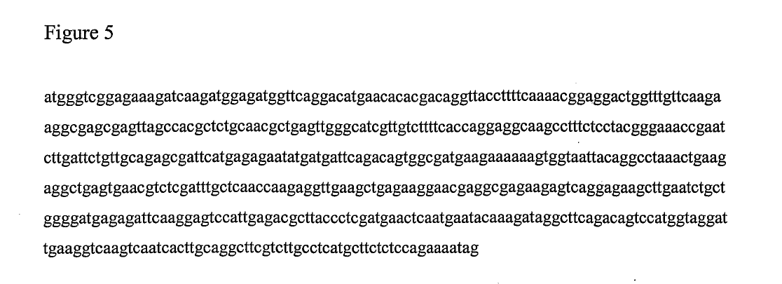

Thus, provided herein are nucleic acids encoding a tocopherol-modulating polypeptide described herein. One example of such an isolated polynucleotide is SEQ ID NO:1 presented in FIG. 1, which sets forth the nucleotide sequence of an Arabidopsis clone identified herein as Ceres clone 19143. Another example of an isolated polynucleotide is SEQ ID NO:24 presented in FIG. 3, which sets forth the nucleotide sequence of an Arabidopsis clone identified herein as Ceres clone 92102. Yet another example of an isolated polynucleotide is SEQ ID NO:31 presented in FIG. 5, which sets forth an Arabidopsis nucleotide sequence identified herein as Ceres cDNA 23495742. Additional examples of nucleic acids encoding tocopherol-modulating polypeptides are set forth SEQ ID NO:16, SEQ ID NO:18, SEQ ID NO:20, SEQ ID NO:22, SEQ ID NO:47, SEQ ID NO:51, SEQ ID NO:56, SEQ ID NO:63, SEQ ID NO:70, SEQ ID NO:74, SEQ ID NO:76, SEQ ID NO:87, SEQ ID NO:92, SEQ ID NO:96, and SEQ ID NO:100. Fragments, fusions, complements, and reverse complements of the described polynucleotides (and encoded polypeptides) also are contemplated.

One or more nucleic acids that encode tocopherol-modulating polypeptides can be used to transform a plant cell such that a plant produced from the plant cell has a modulated (e.g., increased) level of one or both of a tocopherol and a tocotrienol. For example, a nucleic acid encoding a polypeptide that includes an amino acid sequence corresponding to SEQ ID NO:2 can be used to transform a plant cell. A nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:2 can also be used to transform a plant cell.

In certain cases, a nucleic acid encoding a polypeptide that includes an amino acid sequence corresponding to SEQ ID NO:25 can be used to transform a plant cell. In other cases, a nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:25 can be used to transform a plant cell.

In other cases, a nucleic acid encoding a polypeptide that includes an amino acid sequence corresponding to SEQ ID NO:32 can be used to transform a plant cell. In yet other cases, a nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:32 can be used to transform a plant cell.

In certain cases, a nucleic acid encoding a polypeptide that includes an amino acid sequence corresponding to SEQ ID NO:48 can be used to transform a plant cell. In other cases, a nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:48 can be used to transform a plant cell.

In certain cases, a nucleic acid encoding a polypeptide that includes an amino acid sequence corresponding to SEQ ID NO:64 can be used to transform a plant cell. In other cases, a nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:64 can be used to transform a plant cell.

In certain cases, a nucleic acid encoding a polypeptide that includes an amino acid sequence corresponding to SEQ ID NO:77 can be used to transform a plant cell. In other cases, a nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:77 can be used to transform a plant cell.

In certain cases, a nucleic acid encoding a polypeptide that includes an amino acid sequence corresponding to SEQ ID NO:88 can be used to transform a plant cell. In other cases, a nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:88 can be used to transform a plant cell.

In some cases, a nucleic acid encoding a polypeptide that includes an amino acid sequence corresponding to SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:17, SEQ ID NO:19, SEQ ID NO:21, SEQ ID NO:23, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:49, SEQ ID NO:50, SEQ ID NO:52, SEQ ID NO:53, SEQ ID NO:54, SEQ ID NO:55, SEQ ID NO:57, SEQ ID NO:58, SEQ ID NO:59, SEQ ID NO:60, SEQ ID NO:61, SEQ ID NO:62, SEQ ID NO:65, SEQ ID NO:66, SEQ ID NO:67, SEQ ID NO:68, SEQ ID NO:69, SEQ ID NO:71, SEQ ID NO:72, SEQ ID NO:73, SEQ ID NO:75, SEQ ID NO:78, SEQ ID NO:79, SEQ ID NO:80, SEQ ID NO:81, SEQ ED NO:82, SEQ ID NO:83, SEQ ID NO:84, SEQ ID NO:85, SEQ ID NO:86, SEQ ID NO:89, SEQ ID NO:90, SEQ ID NO:91, SEQ ID NO:93, SEQ ID NO:94, SEQ ID NO:95, SEQ ID NO:97, SEQ ID NO:98, SEQ ID NO:99, SEQ ID NO:101, SEQ ID NO:102, the consensus sequence set forth in FIG. 7, the consensus sequence set forth in FIG. 8, the consensus sequence set forth in FIG. 9, the consensus sequence set forth in FIG. 10, the consensus sequence set forth in FIG. 11, the consensus sequence set forth in FIG. 12, or the consensus sequence set forth in FIG. 13 can be used to transform a plant cell.

In some cases, a nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:17, SEQ ID NO:19, SEQ ID NO:21, SEQ ID NO:23, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:49, SEQ ID NO:50, SEQ ID NO:52, SEQ ID NO:53, SEQ ID NO:54, SEQ ID NO:55, SEQ ID NO:57, SEQ ID NO:58, SEQ ID NO:59, SEQ ID NO:60, SEQ ID NO:61, SEQ ID NO:62, SEQ ID NO:65, SEQ ID NO:66, SEQ ID NO:67, SEQ ID NO:68, SEQ ID NO:69, SEQ ID NO:71, SEQ ID NO:72, SEQ ID NO:73, SEQ ID NO:75, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:89, SEQ ID NO:90, SEQ ID NO:91, SEQ ID NO:93, SEQ ID NO:94, SEQ ID NO:95, SEQ ID NO:97, SEQ ID NO:98, SEQ ID NO:99, SEQ ID NO:101, SEQ ID NO:102, the consensus sequence set forth in FIG. 7, the consensus sequence set forth in FIG. 8, the consensus sequence set forth in FIG. 9, the consensus sequence set forth in FIG. 10, the consensus sequence set forth in FIG. 11, the consensus sequence set forth in FIG. 12, or the consensus sequence set forth in FIG. 13 can be used to transform a plant cell.

Two or more nucleic acids that encode tocopherol-modulating polypeptides can also be used to transform a plant cell such that a plant produced from the plant cell has a modulated (e.g., increased) level of one or both of a tocopherol and a tocotrienol. For example, a first nucleic acid encoding a polypeptide that includes an amino acid sequence corresponding to SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID SEQ ID NO:17, SEQ ID NO:19, SEQ ID NO:21, SEQ ID NO:23, or the consensus sequence set forth in FIG. 7, and a second nucleic acid encoding a polypeptide that includes an amino acid sequence corresponding to SEQ ID NO:25, SEQ ID NO:26, SEQ ID NO:27, SEQ ID NO:28, SEQ ID NO:29, SEQ ID NO:30, or the consensus sequence set forth in FIG. 8 can be used to transform a plant cell.

In some cases, a first nucleic acid encoding a polypeptide that includes an amino acid sequence corresponding to SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:17, SEQ ID NO:19, SEQ ID NO:21, SEQ ID NO:23, or the consensus sequence set forth in FIG. 7, and a second nucleic acid encoding a polypeptide that includes an amino acid sequence corresponding to SEQ ID NO:32, SEQ ID NO:33, SEQ ID NO:34, SEQ ID SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, or the consensus sequence set forth in FIG. 9 can be used to transform a plant cell.

In some cases, a first nucleic acid encoding a polypeptide that includes an amino acid sequence corresponding to SEQ ID NO:25, SEQ ID NO:26, SEQ ID NO:27, SEQ ID NO:28, SEQ ID NO:29, SEQ ID NO:30, or the consensus sequence set forth in FIG. 8, and a second nucleic acid encoding a polypeptide that includes an amino acid sequence corresponding SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, or the consensus sequence set forth in FIG. 9 can be used to transform a plant cell.

In other cases, a first nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:17, SEQ ID NO:19, SEQ ID NO:21, SEQ ID NO:23, or the consensus sequence set forth in FIG. 7, and a second nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:25, SEQ ID NO:26, SEQ ID NO:27, SEQ ID NO:28, SEQ ID NO:29, SEQ ID NO:30, or the consensus sequence set forth in FIG. 8 can be used to transform a plant cell.

In other cases, a first nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:17, SEQ ID NO:19, SEQ ID NO:21, SEQ ID NO:23, or the consensus sequence set forth in FIG. 7, and a second nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, or the consensus sequence set forth in FIG. 9 can be used to transform a plant cell.

In yet other cases, a first nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:25, SEQ ID NO:26, SEQ ID NO:27, SEQ ID NO:28, SEQ ID NO:29, SEQ ID NO:30, or the consensus sequence set forth in FIG. 8, and a second nucleic acid encoding a polypeptide having at least 80 percent sequence identity (e.g., 80 percent, 85 percent, 90 percent, 93 percent, 95 percent, 97 percent, 98 percent, or 99 percent sequence identity) to an amino acid sequence corresponding to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, or the consensus sequence set forth in FIG. 9 can be used to transform a plant cell.

It will be appreciated that methods described herein can utilize non-transgenic plant cells or plants that carry a mutation in a tocopherol level-altering polypeptide. For example, a plant carrying a T-DNA insertion, a deletion, a transversion mutation, or a transition mutation in the coding sequence for one of the aforementioned polypeptides can affect tocopherol and/or tocotrienol levels.

As used herein, the term “percent sequence identity” refers to the degree of identity between any given query sequence and a subject sequence. A subject sequence typically has a length that is more than 80%, e.g., more than 82%, 85%, 87%, 89%, 90%, 93%, 95%, 97%, 99%, 100%, 105%, 115%, or 120%, of the length of the query sequence. A percent identity for any query nucleic acid or amino acid sequence, e.g., a tocopherol-modulating polypeptide, relative to another subject nucleic acid or amino acid sequence can be determined as follows. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chema et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For alignment of multiple nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; and gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uklclustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

### Recombinant Constructs, Vectors and Host Cells

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more regulatory regions. Suitable expression vectors include, without limitation, plasmids and viral vectors derived from, for example, bacteriophage, baculoviruses, tobacco mosaic virus, herpesviruses, cytomegalovirus, vaccinia viruses, adenoviruses, adeno-associated viruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The term “regulatory region” refers to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (DTRs), transcriptional start sites, termination sequences, polyadenylation sequences, introns, and other regulatory regions that can reside within coding sequences, such as secretory signals and protease cleavage sites.

As used herein, the term “operably linked” refers to positioning of a regulatory region and a transcribable sequence in a nucleic acid so as to allow or facilitate transcription of the transcribable sequence. For example, a regulatory region is operably linked to a coding sequence when RNA polymerase is able to transcribe the coding sequence into mRNA, which then can be translated into a protein encoded by the coding sequence.

Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element. The choice of promoters to be included depends upon several factors, including, but not limited to, efficiency, selectability, inducibility, desired expression level, and cell or tissue specificity. It is a routine matter for one of skill in the art to modulate expression by appropriately selecting and positioning promoters and other regulatory regions relative to an operably linked sequence. Examples of various classes of promoters are described below. Some of the promoters indicated below are described in more detail in U.S. Patent Application Ser. Nos. 60/505,689; 60/518,075; 60/544,771; 60/558,869; 60/583,609; 60/583,691; 60/612,891; 60/619,181; 60/637,140; 60/757,544; 60/776,307; 110/950,321; 0/957,569; 11/058,689; 11/097,589; 11/172,703; 11/208,308; 11/233,726; 11/274,890; 11/360,017; 11/408,791; 11/414,142; PCT/US05/011105; PCT/US05/034308; and PCT/US05/23639. Nucleotide sequences of regulatory regions are set forth in SEQ ID NOs:103-196. It will be appreciated that a promoter may meet criteria for one classification based on its activity in one plant species, and yet meet criteria for a different classification based on its activity in another plant species.

Constitutive Promoters

Constitutive promoters can promote transcription of an operably linked nucleic acid under most, but not necessarily all, environmental conditions and states of development or cell differentiation. Non-limiting examples of constitutive promoters that can be included in the nucleic acid constructs provided herein include the cauliflower mosaic virus (CaMV) 35S transcription initiation region, the mannopine synthase (MAS) promoter, the 1′ or 2′ promoters derived from T-DNA of Agrobacterium tumefaciens, the figwort mosaic virus 35S promoter, actin promoters such as the rice actin promoter, ubiquitin promoters such as the maize ubiquitin-1 promoter, p32449 (SEQ ID NO:179), and p13879 (SEQ ID NO:177).

Broadly Expressing Promoters

A promoter can be said to be “broadly expressing” when it promotes transcription in many, but not all, plant tissues. For example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. In certain cases, a broadly expressing promoter operably linked to a sequence can promote transcription of the linked sequence in a plant shoot at a level that is at least two times, e.g., at least 3, 5, 10, or 20 times, greater than the level of transcription in a developing seed. In other cases, a broadly expressing promoter can promote transcription in a plant shoot at a level that is at least two times, e.g., at least 3, 5, 10, or 20 times, greater than the level of transcription in a reproductive tissue of a flower. In view of the above, the CaMV 35S promoter is not considered a broadly expressing promoter. Non-limiting examples of broadly expressing promoters that can be included in the nucleic acid constructs provided herein include the p326 (SEQ ID NO:178), YP0158 (SEQ ID NO:159), YP0214 (SEQ ID NO:163), YP0380 (SEQ ID NO:172), PT0848 (SEQ ID NO:128), PT0633 (SEQ ID NO:109), YP0050 (SEQ ID NO:137), YP0144 (SEQ ID NO:157), and YP0190 (SEQ ID NO:161) promoters. See, e.g., U.S. patent application Ser. No. 11/208,308, filed Aug. 19, 2005.

Tissue-, organ- and cell-specific promoters confer transcription only or predominantly in a particular tissue, organ, and cell type, respectively. In some embodiments, promoters specific to vegetative tissues such as the stem, parenchyma, ground meristem, vascular bundle, cambium, phloem, cortex, shoot apical meristem, lateral shoot meristem, root apical meristem, lateral root meristem, leaf primordium, leaf mesophyll, or leaf epidermis can be suitable regulatory regions.

Root-Specific Promoters

Root-specific promoters confer transcription only or predominantly in root tissue. Examples of root-specific promoters include the root specific subdomains of the CaMV 35S promoter (Lam et al., Proc. Natl. Acad. Sci. USA 86:7890-7894 (1989)), root cell specific promoters reported by Conkling et al., Plant Physiol. 93:1203-1211 (1990), and the tobacco RD2 gene promoter.

Seed-Specific Promoters

In some embodiments, promoters that are essentially specific to seeds can be useful. Transcription from a seed-specific promoter occurs primarily in endosperm and cotyledon tissue during seed development. Non-limiting examples of seed-specific promoters that can be included in the nucleic acid constructs provided herein include the napin promoter, the Arcelin-5 promoter, the phaseolin gene promoter (Bustos et al., Plant Cell 1(9):839-853 (1989)), the soybean trypsin inhibitor promoter (Riggs et al., Plant Cell 1(6):609-621 (1989)), the ACP promoter (Baerson et al., Plant Mol. Biol., 22(2):255-267 (1993)), the stearoyl-ACP desaturase gene (Slocombe et al., Plant Plzysiol. 104(4):167-176 (1994)), the soybean α′ subunit of β-conglycinin promoter (Chen et al., Proc. Natl. Acad. Sci. USA 83:8560-8564 (1986)), the oleosin promoter (Hong et al., Plant Mol. Biol. 34(3):549-555 (1997)), zein promoters such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol. 13:5829-5842 (1993)), the beta-amylase gene promoter, and the barley hordein gene promoter.

Non-Seed Fruit Tissue Promoters

Promoters that are active in non-seed fruit tissues can also be useful, e.g., a polygalacturonidase promoter, the banana TRX promoter, the melon actin promoter, YP0396 (SEQ ID NO:176), and PT0623 (SEQ ID NO:196).

Photosynthetically-Active Tissue Promoters

Photosynthetically-active tissue promoters confer transcription only or predominantly in photosynthetically active tissue. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol. 35:773-778 (1994)), the Cab-1 gene promoter from wheat (Fejes et al., Plant Mol. Biol. 15:921-932 (1990)), the CAB-1 promoter from spinach (Lubberstedt et al., Plant Physiol. 104:997-1006 (1994)), the cab1R promoter from rice (Luan et al., Plant Cell 4:971-981 (1992)), the pyruvate, orthophosphate dikinase (PPDK) promoter from corn (Matsuoka et al., Proc. Natl. Acad. Sci. USA 90:9586-9590 (1993)), the tobacco Lhcb1*2 promoter (Cerdan et al., Plant Mol. Biol. 33:245-255 (1997)), the Arabidopsis thaliana SUC2 sucrose-H+ symporter promoter (Truernit et al., Planta 196:564-570 (1995)), and thylakoid membrane protein promoters from spinach (psaD, psaF, psaE, PC, FNR, atpC, atpD, cab, rbcS).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, inducible promoters, such as promoters that confer transcription in response to external stimuli such as chemical agents, developmental stimuli, or environmental stimuli. Other suitable promoters include those set forth in U.S. Patent Application Ser. Nos. 60/505,689; 60/518,075; 60/544,771; 60/558,869; 60/583,691; 60/619,181; 60/637,140; 10/957,569; 11/058,689; 11/172,703 and PCT/US05/23639, e.g., promoters designated YP0086 (gDNA ID 7418340; SEQ ID NO:138), YP0188 (gDNA ID 7418570; SEQ ID NO:160), YP0263 (gDNA ID 7418658; SEQ ID NO:164), PT0758 (SEQ ID NO:124); PT0743 (SEQ ID NO:123); PT0829 (SEQ ID NO:125); YP0096 (SEQ ID NO:141), and YP0119 (SEQ ID NO:151).

Other Regulatory Regions

A 5′ untranslated region (UTR) is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences.

A polyadenylation region at the 3′-end of a coding region can also be operably linked to a coding sequence. The polyadenylation region can be derived from the natural gene, from various other plant genes, or from transfer-DNA (T-DNA).

A suitable enhancer is a cis-regulatory element (−212 to −154) from the upstream region of the octopine synthase (ocs) gene. Fromm et al., The Plant Cell 1:977-984 (1989).

The vectors provided herein also can include, for example, origins of replication, scaffold attachment regions (SARs), and/or markers. A marker gene can confer a selectable phenotype on a plant cell. For example, a marker can confer, biocide resistance, such as resistance to an antibiotic (e.g., kanamycin, G418, bleomycin, or hygromycin), or a herbicide (e.g., glyphosate, chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

It will be understood that more than one regulatory region may be present in a recombinant polynucleotide, e.g., introns, enhancers, upstream activation regions, and inducible elements. Thus, more than one regulatory region can be operably linked to the sequence encoding a tocopherol-modulating polypeptide.

The recombinant DNA constructs provided herein typically include a polynucleotide sequence (e.g., a sequence encoding a tocopherol-modulating polypeptide) inserted into a vector suitable for transformation of plant cells. Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.).

### Transgenic Plants and Cells

The vectors provided herein can be used to transform plant cells and, if desired, generate transgenic plants. Thus, transgenic plants and plant cells containing the nucleic acids described herein also are provided, as are methods for making such transgenic plants and plant cells. A plant or plant cells can be transformed by having the construct integrated into its genome, i.e., can be stably transformed. Stably transformed cells typically retain the introduced nucleic acid sequence with each cell division. Alternatively, the plant or plant cells also can be transiently transformed such that the construct is not integrated into its genome. Transiently transformed cells typically lose some or all of the introduced nucleic acid construct with each cell division, such that the introduced nucleic acid cannot be detected in daughter cells after sufficient number of cell divisions. Both transiently transformed and stably transformed transgenic plants and plant cells can be useful in the methods described herein.

Typically, transgenic plant cells used in the methods described herein constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. Seeds produced by a transgenic plant can be grown and then selfed (or outcrossed and selfed) to obtain seeds homozygous for the nucleic acid construct.

Alternatively, transgenic plant cells can be grown in suspension culture, or tissue or organ culture, for production of secondary metabolites. For the purposes of the methods provided herein, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

Techniques for transforming a wide variety of higher plant species are known in the art. The polynucleotides and/or recombinant vectors described herein can be introduced into the genome of a plant host using any of a number of known methods, including electroporation, microinjection, and biolistic methods. Alternatively, polynucleotides or vectors can be combined with suitable T-DNA flanking regions and introduced into a conventional Agrobacterium tumefaciens host vector. Such Agrobacterium tumefaciens-mediated transformation techniques, including disarming and use of binary vectors, are well known in the art. Other gene transfer and transformation techniques include protoplast transformation through calcium or PEG, electroporation-mediated uptake of naked DNA, electroporation of plant tissues, viral vector-mediated transformation, and microprojectile bombardment (see, e.g., U.S. Pat. Nos. 5,538,880; 5,204,253; 5,591,616; and 6,329,571). If a cell or tissue culture is used as the recipient tissue for transformation, plants can be regenerated from transformed cultures using techniques known to those skilled in the art.

The polynucleotides and vectors described herein can be used to transform a number of monocotyledonous and dicotyledonous plants and plant cell systems, including dicots such as alfalfa, amaranth, apple, beans (including kidney beans, lima beans, green beans), broccoli, cabbage, carrot, castor bean, cherry, chick peas, chicory, clover, cocoa, coffee, cotton, cottonseed, crambe, eucalyptus, flax, grape, grapefruit, lemon, lentils, lettuce, linseed, mango, melon (e.g., watermelon, cantaloupe), mustard, orange, peach, peanut, pear, peas, pepper, plum, poplar, potato, rapeseed (high erucic acid and canola), safflower, sesame, soybean, spinach, strawberry, sugar beet, sunflower, tea, tomato, as well as monocots such as banana, barley, date palm, field corn, garlic, millet, oat, oil palm, onion, pineapple, popcorn, rice, rye, sorghum, sudangrass, sugarcane, sweet corn, switchgrass, turf grasses, and wheat. Gymnosperms such as fir, pine and spruce can also be suitable. Brown seaweeds, green seaweeds, red seaweeds, and microalgae can also be used.

Thus, the methods and compositions described herein can be used with dicotyledonous plants belonging, for example, to the orders Apiales, Arecales, Aristochiales, Asterales, Batales, Campanulctles, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malvales, Myricales, Myrtales, Nymphaeales, Papaverales, Piperales, Plantaginales, Plumbaginales, Podostemales, Polenioniciles, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Bromeliales, Commelinales, Cyclandiales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ginkgoales, Gnetales, and Pinales.

The methods and compositions can be used over a broad range of plant species, including species from the dicot genera Alseodaphne, Amaranthus, Anacardium, Angophora, Apium, Arabidopsis, Arachis, Beta, Bixa, Brassica, Calendula, Camellia, Capsicum, Carthamus, Cicer, Cichorium, Cinnamomum, Citrus, Citrullus, Cocculus, Cocos, Coffea, Corylus, Corymbia, Crambe, Croton, Cucumis, Cucurbita, Cuphea, Daucus, Dianthus, Duguetia, Euphoria, Ficus, Fragaria, Glaucium, Glycine, Glycyrrhiza, Gossypium, Helianthus, Hyoscyamus, Lactuca, Landolphia, Lens, Linum, Litsea, Lupinus, Lycopersicon, Majorana, Maus, Mangifera, Manihot, Medicago, Mentha, Micropus, Nicotiana, Olea, Persea, Petunia, Phaseolus, Pistacia, Pisum, Populus, Prunus, Pyrus, Raphanus, Ricinus, Rosa, Rosmarinus, Rubus, Salix, Senecio, Sesamum, Sinapis, Solanum, Spinacia, Stephania, Tagetes, Theobroma, Trifolium, Trigonella, Vacciniuin, Vicia, Vigna, Vitis; and the monocot genera Agrostis, Allium, Ananas, Andropogon, Asparagus, Avena, Cynodon, Elaeis, Eragrostis, Festuca, Festulolium, Heterocallis, Hordeum, Leinna, Lolium, Musa, Oryza, Panicum, Pennisetum, Phleum, Phoenix, Poa, Saccharum, Secale, Sorghum, Triticum, and Zea; and the gymnosperm genera Abies, Cunninghamia, Picea, Pinus, and Pseudotsuga.

The methods and compositions described herein also can be used with brown seaweeds, e.g., Ascophyllum nodosum, Fucus vesiculosus, Fucus serratus, Himanthalia elongata, and Undaria pinnatifida; red seaweeds, e.g., Chondrus crispus, Cracilaria verrucosa, Porphyra umbilicalis, and Palmaria palmata; green seaweeds, e.g., Enteromorpha spp. and Ulva spp.; and microalgae, e.g., Spirulina sp. (S. platensis and S. maxima) and Odontella aurita. In addition, the methods and compositions can be used with Ciypthecodinium cohnii, Schizochytrium spp., and Haematococcus pluvialis.

In some embodiments, a plant is a member of the species Ananus comosus, Bixa orellana, Brassica campestris, Brassica napus, Brassica oleracea, Calendula officinalis, Chrysanthemum parthenium, Cinnamommum camphora, Coffea arabica, Glycine max, Glycyrrhiza glabra, Gossypium spp., Lactuca sativa, Lycopersicon esculentum, Mentha piperita, Mentha spicata, Musa paradisiaca, Oryza sativa, Rosmarinus officinalis, Solanum tuberosum, Theobroma cacao, Triticum aestivum, Vitis vinifera, or Zea mays.

A transformed cell, callus, tissue, or plant can be identified and isolated by selecting or screening the engineered plant material for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, quantitative real-time PCR, or reverse transcriptase PCR (RT-PCR) amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. After a polynucleotide is stably incorporated into a transgenic plant, it can be introduced into other plants using, for example, standard breeding techniques.

Transgenic plants (or plant cells) can have an altered phenotype as compared to a corresponding control plant (or plant cell) that either lacks the transgene or does not express the transgene. A polypeptide can affect the phenotype of a plant (e.g., a transgenic plant) when expressed in the plant, e.g., at the appropriate time(s), in the appropriate tissue(s), or at the appropriate expression levels. Phenotypic effects can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10% (e.g., less than 9%, 8%, 7%, 6%, 5%, 4%, 3%, 2%, 1%, 0.5%, 0.1%, 0.01%, or 0.001%) of the amount of polypeptide or mRNA encoding the polypeptide exhibited by the plant of interest. Expression can be evaluated using methods including, for example, quantitative real-time PCR, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-specific or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

A population of transgenic plants can be screened and/or selected for those members of the population that have a desired trait or phenotype conferred by expression of the transgene. Selection and/or screening can be carried out over one or more generations, which can be useful to identify those plants that have a desired trait, such as an increased tocopherol content. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is exhibited by the plant.

When a tocopherol-modulating polypeptide described herein is expressed in a transgenic plant, the plant can have altered (e.g., increased) levels of one or both of a tocopherol and a tocotrienol. The level of one or both of a tocopherol and a tocotrienol can be altered in the seed of the transgenic plant and/or in the non-seed tissue of the transgenic plant. A tocopherol can be α-, β-, δ-, or γ-tocopherol. A tocotrienol can be α-, β-, δ-, or γ-tocotrienol. Thus, a transgenic plant expressing one or more tocopherol-modulating polypeptides can have an increased level of one or more of α-tocopherol, β-tocopherol, δ-tocopherol, γ-tocopherol, α-tocotrienol, β-tocotrienol, δ-tocotrienol, and γ-tocotrienol, and the increased level can be in the seed and/or the non-seed tissue.

For example, in certain embodiments, seeds of a transgenic plant can exhibit increased levels of α-tocopherol, γ-tocopherol, α-tocotrienol, and/or γ-tocotrienol. In some embodiments, non-seed tissues of a transgenic plant can exhibit increased levels of β-tocopherol, δ-tocopherol, β-tocotrienol, and/or δ-tocotrienol. A tocotrienol level can be increased by at least 5 percent (e.g., 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 800, 900, 950, 1000, 1100, 1200, 1300, 1400, 1500, 1600, 1650, 1700, 1800, 1900, 2000, 2100, 2200, 2300, 2400, or 2500 percent) as compared to a tocotrienol level in a corresponding control plant that does not express the transgene. A tocopherol level can be increased by at least 5 percent (e.g., 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 800, 900, 1000, 1100, 1200, 1300, 1400, 1500, 1600, 1700, 1800, 1900, 2000, 2100, 2200, 2300, 2400, or 2500 percent) as compared to a tocopherol level in a corresponding control plant that does not express the transgene. For example, a level of β- and/or δ-tocopherol in the non-seed tissues of a plant can be increased by at least 20% to about 2500% or any value therebetween, such as at least 21%, 22%, 30%, 32%, 37%, 45%, 52%, 58%, 65%, 73%, 80%, 85%, 100%, 210%, 300%, 380%, 394%, 400%, 460%, 500%, 549%, 600%, 670%, 700%, 800%, 840%, 940%, 990%, 1050%, 1100%, 1200%, 1300%, 1400%, 1500%, 1600%, 1700%, 1800%, 1900%, 2000%, 2100%, 2200%, 2300%, 2400%, or 2490%, as compared to the corresponding levels in a control plant. A level of α-tocopherol in the seeds of a plant can be increased by at least 20% to about 2500% or any value therebetween, such as at least 25%, 32%, 55%, 75%, 100%, 175%, 250%, 300%, 400%, 500%, 600%, 700%, 745%, 800%, 836%, 900%, 950%, 1000%, 1100%, 1200%, 1300%, 1400%, 1500%, 1600%, 1700%, 1800%, 1900%, 2000%, 2100%, 2200%, 2300%, 2400%, or 2495%, as compared to the corresponding levels in a control plant.

### Seeds, Oils, Vegetative Tissues, Animal Feed, and Articles of Manufacture

Transgenic plants provided herein have particular uses in the agricultural and nutritional industries, e.g., in compositions such as food and feed products.

Seeds of transgenic plants describe herein can be conditioned and bagged in packaging material by means known in the art to form an article of manufacture. Packaging material such as paper and cloth are well known in the art. Such a bag of seed preferably has a package label accompanying the bag, e.g., a tag or label secured to the packaging material, a label printed on the packaging material or a label inserted within the bag. The package label may indicate the seed contained therein incorporates transgenes that provide increased amounts of one or more tocopherols in one or more tissues of plants grown from such seeds.

Transgenic plants described herein can be used to make food products such as fresh, frozen, or canned vegetables and fruits. Suitable plants with which to make such products include bananas, broccoli, grapes, lettuce, mango, melon, spinach, strawberry and tomatoes. Such products are useful to provide increased amounts of tocopherol(s) in a human diet.

Seeds from transgenic plants described herein can be used to make food products such as flours, vegetable oils and insoluble fibers. In particular, refined, bleached, and deodorized vegetable oils are useful because they can provide an increased tocopherol content to a human diet and have increased oxidative stability. Suitable plants from which to make such vegetable oils include soybean, canola, corn, cottonseed, flax, oil palm, safflower, and sunflower. Such oils can be used for flying, baking, and spray coating applications.

Seeds from transgenic plants described herein can also be used to make industrial lubricants such hydraulic fluids, engine and transmission oils, cutting oils, transformer fluids, and turbine oil base stocks. A refined, bleached, and deodorized vegetable oil having high oleic acid and low linolenic acid contents is useful because an increased tocopherol content in such an oil can increase the oxidative stability relative to a high oleic acid and low linolenic acid vegetable oil from corresponding control plants. In certain cases, a vegetable oil from seeds of transgenic plants described herein can exhibit an increased level of one or more tocopherols, such as an increased level of α-tocopherol and/or γ-tocopherol. Suitable plants from which to make such vegetable oils include soybean, canola, corn, cottonseed, sunflower, coconut or palm.

Seeds or non-seed tissues from transgenic plants described herein can also be used as a source from which to extract tocopherols and/or tocotrienols using techniques known in the art, e.g., extraction with an organic solvent such as hexane. The resulting extract can be included in nutritional supplements as well as processed food products, e.g., snack products, frozen entrees, vegetable oils, breakfast cereals, and baby foods.

### Methods

Also provided herein are methods that employ the described polynucleotides, plant cells, transgenic plants, seeds, and tissues. For example, a method of modulating the level of one or both of a tocopherol and a tocotrienol in a plant, such as in non-seed tissue or seeds of a plant, is provided. The method includes introducing an exogenous nucleic acid comprising a polynucleotide sequence described herein into a plant cell. A modulated level can be an increased level of a tocopherol, including one or more of α, γ, β and/or δ tocopherol and one or more of α-, β-, δ-, and/or γ-tocotrienol.

A method of producing a plant having seed with an increased level of one or both of a tocopherol and a tocotrienol (e.g., an increased α-tocopherol, γ tocopherol, α-tocotrienol, and/or γ-tocotrienol level) is also provided, which includes introducing into a plant cell an exogenous nucleic acid as previously described, and growing a plant from the plant cell. Similarly, a method of producing a plant having non-seed tissue with an increased level of one or both of a tocopherol and a tocotrienol (e.g., an increased β-tocopherol, δ-tocopherol, β-tocotrienol, and/or δ-tocotrienol level) is also provided, which includes introducing into a plant cell an exogenous nucleic acid as previously described, and growing a plant from the plant cell. Finally, a method of producing an oil having an increased oxidative stability in the absence of added antioxidants is provided. Such a method includes extracting and processing oil from seed of a transgenic plant described herein. Suitable oil processing techniques are known. See, e.g., Bailey's Industrial & Fat Products, Volume 2, Hui, Y. H., ed., 5th edition, Wiley and Sons, New York (1996).

The invention will be further described in the following examples, which do not limit the scope of the invention described in the claims.

### EXAMPLES

### Example 1

### Transgenic Plants

The following symbols are used in the Examples: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

Ceres clone 19143 (SEQ ID NO:1) encodes a 338 amino acid (SEQ ID NO:2) putative chloroplast inner envelope protein from Arabidopsis predicted to be an MPBQ/MSBQ methyltransferase. Ceres clone 92102 (SEQ ID NO:24) encodes a 241 amino acid DNA binding protein-like polypeptide (SEQ ID NO:25) from Arabidopsis. Ceres cDNA 23495742 (SEQ ID NO:31) encodes a 172 amino acid MADS-box family polypeptide (SEQ ID NO:32) from Arabidopsis. Ceres ANNOT ID 567302 (SEQ ID NO:47) encodes a 488 amino acid tocopherol cyclase 1 polypeptide (SEQ ID NO:48) from Arabidopsis. Ceres ANNOT ID 552252 (SEQ ID NO:63) encodes a 393 amino acid homogentisate phytylprenyltransferase polypeptide (SEQ ID NO:64) from Arabidopsis. Ceres ANNOT ID no. 859061 (SEQ ID NO:76) encodes a 174 amino acid polypeptide (SEQ ID NO:77) from Arabidopsis. Ceres CLONE ID no. 125255 (SEQ ID NO:87) encodes a 304 amino acid polypeptide (SEQ ID NO:88) from Arabidopsis.

Ti plasmid vectors were constructed that contained Ceres clone 19143, Ceres clone 92102, Ceres cDNA 23495742, Ceres ANNOT ID 567302, Ceres ANNOT ID 552252, Ceres ANNOT ID no. 859061, or Ceres CLONE ID no. 125255 operably linked to the 35S promoter. The Ti plasmid vector used for these constructs, CRS 338, contained a phosphinothricin acetyltransferase gene, which confers Finale™ resistance to transformed plants. Wild-type Arabidopsis Wassilewskija (Ws) plants were transformed separately with each Ti plasmid vector, essentially as described in Bechtold et al., C.R. Acad. Sci. Paris, 316:1194-1199 (1993).

Arabidopsis lines containing Ceres clone 19143, Ceres clone 92102, Ceres cDNA 23495742, Ceres ANNOT ID 567302, Ceres ANNOT ID 552252, Ceres ANNOT ID no. 859061, or Ceres CLONE ID no. 125255 were designated ME06634, ME04024, ME10864, ME10540, ME10499, ME23450, or ME07198, respectively. The presence of the Ceres clone 19143 vector in ME06634, the Ceres clone 92102 vector in ME04024, the Ceres cDNA 23495742 vector in ME10864, Ceres ANNOT ID 567302 vector in ME10540, the Ceres ANNOT ID 552252 vector in ME10499, the Ceres ANNOT ID no. 859061 vector in ME23450, and the Ceres CLONE ID no. 125255 vector in ME07198 was confirmed by Finale™ resistance, PCR amplification from green leaf tissue extract, and sequencing of PCR products.

As controls, wild-type Arabidopsis Wassilewskija (Ws) plants were transformed with the empty vector CRS 338, generating plant line SR00559.

Ten events of each of ME06634, ME04024, and ME10499; seven events of ME10864; and five events of ME10540 were selected and screened for visible phenotypic alterations in the T₁ generation.

The physical appearance of eight of the ten T₁ ME06634 plants was identical to the physical appearance of the controls. Events -01 and -03 of ME06634 were green as seedlings, but they developed yellowing leaves as they matured.

The physical appearance of nine of the ten T₁ ME04024 plants was identical to that of the control plants. Event -03 of ME04024 appeared smaller and had increased branching. This phenotype is typically seen when a plant is injured during the T₁ weeding out process. Therefore, it is likely that this phenotype was not related to expression of the transgene.

The physical appearance of all T₁ ME10499, ME10864, and ME10540 plants was identical to that of the control plants.

### Example 2

### Analysis of Tocopherol Levels in ME06634 Events

Seeds from each of four events of ME06634 were planted separately. T₂ and T₃ plants from each of the four events of ME06634 were grown until ten days post-bolting. Aerial tissues from four Finale™-resistant plants of each event were pooled, frozen in liquid nitrogen, and stored at −80° C. The frozen tissues were lyophilized for 72 hours and stored at −80° C. The freeze-dried tissues were crushed into a fine powder and prepared for analysis using gas chromatography-mass spectroscopy (GC-MS). Briefly, 30 mg of the lyophilized plant tissues were extracted with ethyl acetate. The resulting extract was dried and derivatized using N-Methyl-N-(trimethylsilyl)trifluoroacetamide (MSTFA) in pyridine. Sterols and tocopherols in the derivatized extract were separated and detected using GC-MS.

The GC-MS analysis showed that Finale™-resistant T₂ plants from events -02 and -03 had significantly increased and δ- and β-tocopherol levels compared to control plants. As presented in Table 1, δ-tocopherol levels were increased to 494% and 560% in events -02 and -03, respectively, compared to the corresponding control plants. As presented in Table 2, β-tocopherol levels were increased to 940% and 770% in events -02 and -03, respectively, compared to the corresponding control plants. Only plants not showing a yellowing phenotype, as described in Example 1, were used for analysis.

TABLE 1
δ-Tocopherol levels (% Control) in T₂ and T₃ plants
from ME06634 events

| | Event-02 | Event-03 | Event-05 | Event-06 | Control |
|---|---|---|---|---|---|
| T₂ | 494 ± 27 | 560 ± 78 | 42 ± 6 | 33 ± 2 | 100 ± 27 |
| p-value | <0.01 | <0.01 | <0.01 | <0.01 | NA |
| T₃ | 130 ± 15 | 480 ± 5 | 72 ± 23 | 42 ± 12 | 100 ± 32 |
| p-value | <0.01 | <0.01 | 0.17 | 0.34 | NA |

TABLE 2
β-Tocopherol levels (% Control) in T₂ and
T₃ plants from ME06634 events

| | Event-02 | Event-03 | Event-05 | Event-06 | Control |
|---|---|---|---|---|---|
| T₂ | 940 ± 83 | 770 ± 200 | 41 ± 5 | 26 ± 22 | 100 ± 11 |
| p-value | <0.01 | 0.02 | <0.01 | <0.01 | NA |
| T₃ | 120 ± 31 | 310 ± 50 | 60 ± 25 | 31 ± 11 | 100 ± 20 |
| p-value | 0.01 | <0.01 | 0.1 | 0.34 | NA |

Levels of δ- and β-tocopherol in Finale™-resistant T₃ plants from four ME06634 events also were analyzed using GC-MS. Events -02 and -03 had significantly increased δ- and β-tocopherol levels compared to control plants. As presented in Table 1, β-tocopherol levels were increased to 130% and 480% in events -02 and -03, respectively, compared to the corresponding control plants. As presented in Table 2, β-tocopherol levels were increased to 120% and 310% in events -02 and -03, respectively, compared to the corresponding control plants.

Tocopherol levels in seeds from T₃ plants of four ME06634 events were also analyzed by GC-MS. Event -02 had a significantly increased level of α-tocopherol compared to control plants. As presented in Table 3, the level of α-tocopherol was increased to 936% in event -02 compared to the corresponding control plants.

TABLE 3
α-Tocopherol levels (% Control) in seeds from
T₃ plants from ME06634 events

| | Event-02 | Event-03 | Event-05 | Event-06 | Control |
|---|---|---|---|---|---|
| T₃ | 936 ± 189 | 105 ± 15 | 125 ± 10 | 132 ± 1 | 100 ± 8 |
| p-value | 0.10 | 0.74 | 0.06 | 0.37 | NA |

Further experiments were conducted to look for changes in other metabolites in ME06634. These studies showed the following:

a. α- and γ-tocopherol did not change significantly in aerial tissue.

b. No other statistically significant changes were detected by visual inspection of the chromatograms of aerial tissue extracts of T₂ or T₃ plants from ME06634 events.

c. There was a decrease in both β- and δ-tocopherol levels in aerial tissues of event -06 over two generations. There was also a decrease in both β- and δ-tocopherol in event -05 in the T₂ generation and a lower level of both, although not significantly lower, in the T₃ generation.

T₂ plants from events -02 and -03 of ME06634 were analyzed for morphology. Starting at close to the time of flowering, the plants exhibited the same progressive yellowing phenotype that was observed in the T₁ generation, but in a recessive segregation pattern. This suggested that the phenotype was gene-dosage dependent and would be mitigated in appropriately expressing plants. Since this yellowing was observed in two T₁ and in two T₂ plants (and in a recessive pattern), it seemed highly unlikely that it could be due to a dominant change-of-function mutation. In fact, there were degrees of severity in the plants that exhibited the phenotype.

There was no detectable reduction in germination rate in T₂ plants from ME06634. The general morphology/architecture appeared wild-type in all instances, except as noted above. There were no observable or statistically significant differences between experimental plants and control plants in days to flowering or rosette area seven days post-bolting. There were no observable or statistical differences between non-yellowing experimental plants that displayed the chemotype and control plants with regard to fertility (silique number and seed fill).

A calibration curve was generated using various concentrations of a 5-tocopherol standard. The δ-tocopherol concentrations in the samples were within the quantifiable range of the assay.

### Example 3

### Analysis of Tocopherol Levels in ME04024 Events

Seeds from each of four events of ME04024 were planted separately. T₂ and T₃ plants from each of the four events of ME04024 were grown until ten days post-bolting. Aerial tissues from four Finale™-resistant plants of each event were analyzed using GC-MS as described above.

The GC-MS analysis showed that Finale™-resistant T₂ plants from events -04 and -05 of ME04024 had significantly increased δ-tocopherol levels compared to control plants. As presented in Table 4, δ-tocopherol levels were increased to 137% and 152% in events -04 and -05, respectively, compared to the corresponding control plants.

TABLE 4
δ-Tocopherol levels (% Control) in T₂ and
T₃ plants from ME04024 events

| | Event-01 | Event-02 | Event-04 | Event-05 | Control |
|---|---|---|---|---|---|
| T₂ | 80 ± 15 | 127 ± 13 | 137 ± 13 | 152 ± 9 | 100 ± 6 |
| p-value | 0.14 | 0.05 | 0.02 | <0.01 | NA |
| T₃ | 83 ± 11 | 103 ± 19 | 122 ± 8 | 121 ± 9 | 100 ± 11 |
| p-value | 0.12 | 0.84 | 0.02 | 0.03 | NA |

Levels of δ-tocopherol in Finale™-resistant T₃ plants from four ME04024 events also were analyzed using GC-MS. Events -04 and -05 had significantly increased δ-tocopherol levels compared to control plants. As presented in Table 4, δ-tocopherol levels were increased to 122% and 121% in events -04 and -05, respectively, compared to the corresponding control plants.

Additional experiments were conducted to test for changes in the levels of other metabolites in ME04024. Results of these experiments indicated that α-, β-, and γ-tocopherol levels did not change significantly. Furthermore, no other statistically significant changes were detected by visual inspection of the chromatograms of the extracts from T₂ or T₃ plants from ME04024 events.

There were no observable or statistically significant differences between T₂ ME04024 and control plants in germination, onset of flowering, rosette area, fertility, and general morphology/architecture.

A calibration curve was generated using various concentrations of a 5-tocopherol standard. The δ-tocopherol concentrations in the samples were within the quantifiable range of the assay.

### Example 4

### Analysis of Tocopherol Levels in ME10864 Events

Seeds from each of five events of ME10864 were planted separately. T₂ plants from each of the five events were grown until ten days post-bolting. Aerial tissues from four Finale™-resistant plants of each event were analyzed using GC-MS as described above.

The GC-MS analysis showed that Finale™-resistant T₂ plants from events -04 and -05 of ME10864 had significantly increased δ-tocopherol levels compared to control plants. As presented in Table 5, δ-tocopherol levels were increased to 649% and 165% in events -04 and -05, respectively, compared to the corresponding control plants.

TABLE 5
δ-Tocopherol levels (% Control) in T₂ and
T₃ plants from ME10864 events

| | Event- | | Event- | | | |
|---|---|---|---|---|---|---|
| | 01 | Event-02 | 03 | Event-04 | Event-05 | Control |
| T₂ | 115 ± | 114 ± 21 | 146 ± | 649 ± 60 | 165 ± 43 | 100 ± 42 |
| | 5 | | 19 | | | |
| p- | 0.58 | 0.61 | 0.11 | <0.01 | 0.05 | N/A |
| value | | | | | | |
| T₃ | NA | 107 ± 0 | NA | 132 ± 26 | 185 ± 14 | 100 ± 17 |
| p- | NA | 0.52 | NA | 0.03 | <0.01 | N/A |
| value | | | | | | |

Levels of δ-tocopherol in Finale™-resistant T₃ plants from three ME10864 events were also analyzed using GC-MS. Events -04 and -05 had significantly increased δ-tocopherol levels as compared to control plants. As presented in Table 5, δ-tocopherol levels were increased to 132% and 185% in events -04 and -05, respectively, compared to the corresponding control plants.

Further experiments were conducted to test for changes in the levels of other metabolites in ME10864. The results of these studies were as follows:

a. The level of β-tocopherol increased by 430% in T₂ plants from event -04 compared to control plants. However, the level of β-tocopherol in T₃ plants from event -04 was not significantly different from control plants.

b. No other statistically significant changes were detected by visual inspection of the chromatograms of the extracts from T₂ or T₃ plants from ME10864 events.

There were no observable or statistically significant differences between T₂ ME10864 and control plants in germination, onset of flowering, rosette area, fertility, and general morphology/architecture.

A calibration curve was generated using various concentrations of a δ-tocopherol standard. The δ-tocopherol concentrations in the samples were within the quantifiable range of the assay.

### Example 5

### Analysis of Tocopherol Levels in ME10540 Events

Seeds from each of five events of ME10540 were planted separately. T₂ and T₃ plants from each of the five events were grown until ten days post-bolting. Aerial tissues from four Finale™-resistant plants of each event were analyzed using GC-MS as described above.

The GC-MS analysis showed that Finale™-resistant T₂ plants from events -02, -03, and -04 of ME10540 had significantly increased α- and γ-tocopherol levels compared to control plants. As presented in Table 6, α-tocopherol levels were increased to 203%, 173%, and 192% in events -02, -03, and -04, respectively, compared to the corresponding control plants. As presented in Table 7, γ-tocopherol levels were increased to 169%, 171%, and 188% in events -02, -03, and -04, respectively, as compared to the corresponding control plants.

T₂ plants from events -01 and -05 of ME10540 had significantly decreased γ-tocopherol levels compared to control plants. As presented in Table 7, γ-tocopherol levels were decreased to 20% and 35% in events -01 and -05, respectively, compared to control plants.

TABLE 6
α-Tocopherol levels (% Control) in T₂ and
T₃ plants from ME10540 events

| | | | | | Event- | |
|---|---|---|---|---|---|---|
| | Event-01 | Event-02 | Event-03 | Event-04 | 05 | Control |
| T₂ | 34 ± 4 | 203 ± 7 | 173 ± 18 | 192 ± 26 | 41 ± 6 | 100 ± |
| | | | | | | 68 |
| p- | 0.28 | 0.01 | 0.04 | 0.02 | 0.36 | N/A |
| value | | | | | | |
| T₃ | 110 ± 6 | 122 ± 4 | 132 ± 4 | 132 ± 8 | 97 ± 2 | 100 ± |
| | | | | | | 4 |
| p- | <0.01 | <0.01 | <0.01 | <0.01 | 0.55 | N/A |
| value | | | | | | |

TABLE 7
γ-Tocopherol levels (% Control) in T₂ and
T₃ plants from ME10540 events

| | Event- | | | | Event- | |
|---|---|---|---|---|---|---|
| | 01 | Event-02 | Event-03 | Event-04 | 05 | Control |
| T₂ | 20 ± 3 | 169 ± 8 | 171 ± 23 | 188 ± 23 | 35 ± 3 | 100 ± 57 |
| p- | 0.03 | 0.04 | 0.04 | 0.02 | 0.03 | N/A |
| value | | | | | | |
| T₃ | 101 ± 4 | 134 ± 7 | 130 ± 5 | 137 ± 12 | 90 ± 6 | 100 ± 4 |
| p- | 0.49 | <0.01 | <0.01 | <0.01 | 0.21 | N/A |
| value | | | | | | |

Levels of α- and γ-tocopherol in Finale™-resistant T₃ plants from five events of ME10540 also were analyzed using GC-MS. Events -02, -03, and -04 had significantly increased α- and γ-tocopherol levels compared to control plants. As presented in Table 6, α-tocopherol levels were increased to 122% in event -02, and to 132% in events -03 and -04, compared to the control plants. As presented in Table 7, γ-tocopherol levels were increased to 134%, 130%, and 137% in events -02, -03, and -04, respectively, compared to the control plants.

The α-tocopherol level in event -01 also was significantly increased compared to control plants. As presented in Table 6, the α-tocopherol level was increased to 110% in event -01 compared to control plants.

Levels of β- and δ-tocopherol in aerial tissues from four Finale™-resistant T₂ plants of each of four events of ME10540 also were analyzed using GC-MS. Events -02, -03, and -04 had significantly increased levels of β- and δ-tocopherol compared to control plants. As presented in Table 8, β-tocopherol levels were increased to 781%, 894%, and 937% in events -02, -03, and -04, respectively, compared to the control plants. As presented in Table 9, δ-tocopherol levels were increased to 432%, 447%, and 543% in events -02, -03, and -04, respectively, compared to the corresponding control plants.

The β-tocopherol level in event -05 also was significantly increased compared to control plants. As presented in Table 8, the β-tocopherol level was increased to 223% in event -05 compared to control plants.

TABLE 8
β-Tocopherol levels (% Control) in T₂ and
T₃ plants from ME10540 events

| | Event-02 | Event-03 | Event-04 | Event-05 | Control |
|---|---|---|---|---|---|
| T₂ | 781 ± 96 | 894 ± 183 | 937 ± 166 | 223 ± 36 | 100 ± 63 |
| p-value | <0.01 | <0.01 | <0.01 | <0.01 | N/A |
| T₃ | 625 ± 15 | 1199 ± 45 | 917 ± 63 | 509 ± 27 | 100 ± 9 |
| p-value | <0.01 | <0.01 | <0.01 | <0.01 | N/A |

TABLE 9
δ-Tocopherol levels (% Control) in T₂ and
T₃ plants from ME10540 events

| | Event-02 | Event-03 | Event-04 | Event-05 | Control |
|---|---|---|---|---|---|
| T₂ | 432 ± 21 | 447 ± 54 | 543 ± 36 | 132 ± 12 | 100 ± 63 |
| p-value | <0.01 | <0.01 | <0.01 | 0.09 | N/A |
| T₃ | 376 ± 10 | 720 ± 37 | 530 ± 15 | 301 ± 25 | 100 ± 8 |
| p-value | <0.01 | <0.01 | <0.01 | <0.01 | N/A |

Levels of β- and δ-tocopherol in Finale™-resistant T₃ plants from four ME10540 events also were analyzed using GC-MS. Events -02, -03, -04, and -05 had significantly increased levels of β- and δ-tocopherol compared to control plants. As presented in Table 8, β-tocopherol levels were increased to 625%, 1199%, 917%, and 509% in events -02, -03, -04, and -05, respectively, compared to control plants. As presented in Table 9, δ-tocopherol levels were increased to 376%, 720%, 530%, and 301% in events -02, -03, -04, and -05, respectively, compared to control plants.

Further studies were conducted to look for changes in other metabolites in ME10540. No other statistically significant changes were detected by visual inspection of the chromatograms of aerial tissue extracts of T₂ or T₃ plants from ME10540 events.

There were no observable or statistically significant differences between T₂ ME10540 and control plants in germination, onset of flowering, rosette area, fertility, and general morphology/architecture.

Calibration curves were generated using α-, β-, γ-, and γ-tocopherol standards. The measured tocopherol levels were within the quantifiable range of the assay.

### Example 6

### Analysis of Tocopherol Levels in ME10499 Events

Seeds from each of five events of ME10499 were planted separately. T₂ and T₃ plants from five and four events, respectively, of ME10499 were grown until ten days post-bolting. Aerial tissues from four Finale™-resistant plants of each event were analyzed using GC-MS as described above.

The GC-MS analysis showed that Finale™-resistant T₂ plants from events -01, -04, and -05 of ME10499 had significantly increased α- and γ-tocopherol levels compared to control plants. As presented in Table 10, α-tocopherol levels were increased to 155%, 131%, and 211% in events -01, -04, and -05, respectively, compared to the corresponding control plants. As presented in Table 11, γ-tocopherol levels were increased to 224%, 242%, and 373% in events -01, -04, and -05, respectively, as compared to the corresponding control plants.

T₂ plants from events -02 and -03 had significantly decreased α- and tocopherol levels compared to control plants. As presented in Table 10, α-tocopherol levels were decreased to 45% and 39% in events -02 and -03, respectively, compared to control plants. As presented in Table 11, γ-tocopherol levels were decreased to 55% and 68% in events -02 and -03, respectively, compared to control plants.

TABLE 10
α-Tocopherol levels (% Control) in T₂ and
T₃ plants from ME10499 events

| | Event- | Event- | Event- | | | |
|---|---|---|---|---|---|---|
| | 01 | 02 | 03 | Event-04 | Event-05 | Control |
| T₂ | 155 ± 8 | 45 ± 0 | 39 ± 1 | 131 ± 3 | 211 ± 14 | 100 ± 13 |
| p-value | <0.01 | <0.01 | <0.01 | <0.01 | <0.01 | N/A |
| T₃ | 183 ± 3 | ND* | 169 ± 1 | 159 ± 2 | 219 ± 3 | 100 ± 54 |
| p-value | <0.01 | ND* | <0.01 | 0.01 | <0.01 | N/A |

*ND = not determined

TABLE 11
γ-Tocopherol levels (% Control) in T₂ and
T₃ plants from ME10499 events

| | Event- | Event- | | Event- | | |
|---|---|---|---|---|---|---|
| | 01 | 02 | Event-03 | 04 | Event-05 | Control |
| T₂ | 224 ± 9 | 55 ± 3 | 68 ± 5 | 242 ± 6 | 373 ± 28 | 100 ± 15 |
| p-value | <0.01 | <0.01 | <0.01 | <0.01 | <0.01 | N/A |
| T₃ | 220 ± 1 | ND* | 190 ± 13 | 195 ± 1 | 303 ± 12 | 100 ± 58 |
| p-value | <0.01 | ND* | <0.01 | <0.01 | <0.01 | N/A |

*ND = not determined

Levels of α- and γ-tocopherol in Finale™-resistant T₃ plants from four events of ME10499 also were analyzed using GC-MS. Events -01, -03, -04, and -05 had significantly increased α- and γ-tocopherol levels compared to control plants. As presented in Table 10, α-tocopherol levels were increased to 183%, 169%, 159%, and 219% in events -01, -03, -04, and -05, respectively, compared to control plants. As presented in Table 11, γ-tocopherol levels were increased to 220%, 190%, 195%, and 303% in events -01, -03, -04, and -05, respectively, compared to control plants.

Levels of α-tocopherol in aerial tissues of Finale™-resistant T₂ plants from five events of ME10499 also were analyzed using GC-MS. Events -01, -02, -03, -04, and -05 had significantly increased levels of δ-tocopherol compared to control plants. As presented in Table 12, δ-tocopherol levels were increased to 306%, 337%, 576%, 421%, and 686% in events -01, -02, -03, -04, and -05, respectively, compared to control plants.

TABLE 12
δ-Tocopherol levels (% Control) in T₂ and
T₃ plants from ME10499 events

| | | Event- | Event- | | | |
|---|---|---|---|---|---|---|
| | Event-01 | 02 | 03 | Event-04 | Event-05 | Control |
| T₂ | 306 ± 9 | 337 ± | 576 ± | 421 ± 15 | 686 ± 23 | 100 ± 29 |
| | | 20 | 28 | | | |
| p- | <0.01 | <0.01 | <0.01 | <0.01 | <0.01 | N/A |
| value | | | | | | |
| T₃ | 84 ± 11 | ND* | 92 ± 7 | 116 ± 6 | 156 ± 13 | 100 ± 61 |
| p- | 0.66 | ND* | 0.89 | 0.38 | 0.03 | N/A |
| value | | | | | | |

*ND = not determined

Levels of δ-tocopherol in Finale™-resistant T₃ plants from four ME10499 events also were analyzed using GC-MS. Event -05 had a significantly increased level of δ-tocopherol compared to control plants. As presented in Table 12, the δ-tocopherol level was increased to 156% in event -05 compared to the corresponding control plants.

Further studies were conducted to look for changes in other metabolites in ME10499. No other statistically significant changes were detected by visual inspection of the chromatograms of aerial tissue extracts of T₂ or T₃ plants from ME10499 events.

There were no observable or statistically significant differences between T₂ ME10499 and control plants in germination, onset of flowering, rosette area, fertility, and general morphology/architecture.

Calibration curves were generated using α- and γ-tocopherol standards. The measured tocopherol levels were within the quantifiable range of the assay.

Segregation analysis of T₂ seedlings from ME10499 events based on Finale™ resistance showed a 3:1 ratio of resistant to sensitive for event -01, and a 15:1 ratio of resistant to sensitive for event -05.

### Example 7

### Analysis of Tocopherol Levels in ME23450 Events

Seeds from three events of ME23450 were planted separately. T₂ plants from each of the three events of ME23450 were grown until ten days post-bolting. Aerial tissues from Finale™-resistant plants of each event were analyzed using GC-MS as described above.

The GC-MS analysis showed that Finale™-resistant T₂ plants from events -02, -03, and -04 of ME23450 had significantly increased α-, β-, δ- and γ-tocopherol levels compared to control plants. As presented in Table 13, α-tocopherol levels were increased to 128%, 139%, and 131% in events -02, -03, and -04, respectively, compared to control plants. As presented in Table 14, β-tocopherol levels were increased to 168%, 194%, and 193% in events -02, -03, and -04, respectively, compared to control plants. As presented in Table 15, δ-tocopherol levels were increased to 294%, 454%, and 653% in events -02, -03, and 04, respectively, compared to control plants. As presented in Table 16, γ-tocopherol levels were increased to 175%, 198%, and 196% in events -02, -03, and -04, respectively, compared to control plants.

TABLE 13
α-Tocopherol levels (% Control) in T₂ plants from ME23450 events

| | Event-02 | Event-03 | Event-04 | Control |
|---|---|---|---|---|
| T₂ | 128 ± 1 | 139 ± 1 | 131 ± 4 | 100 ± 19 |
| p-value | <0.01 | <0.01 | <0.01 | N/A |

TABLE 14
β-Tocopherol levels (% Control) in T₂ plants from ME23450 events

| | Event-02 | Event-03 | Event-04 | Control |
|---|---|---|---|---|
| T₂ | 168 ± 14 | 194 ± 14 | 193 ± 10 | 100 ± 31 |
| p-value | <0.01 | <0.01 | <0.01 | N/A |

TABLE 15
δ-Tocopherol levels (% Control) in T₂ plants from ME23450 events

| | Event-02 | Event-03 | Event-04 | Control |
|---|---|---|---|---|
| T₂ | 294 ± 17 | 454 ± 37 | 653 ± 23 | 100 ± 28 |
| p-value | <0.01 | <0.01 | <0.01 | N/A |

TABLE 16
γ-Tocopherol levels (% Control) in T₂ plants from ME23450 events

| | Event-02 | Event-03 | Event-04 | Control |
|---|---|---|---|---|
| T₂ | 175 ± 1 | 198 ± 17 | 196 ± 4 | 100 ± 21 |
| p-value | <0.01 | <0.01 | <0.01 | N/A |

### Example 8

### Analysis of Tocopherol Levels in ME07198 Events

Seeds from each of five events of ME07198 were planted separately. T₂ plants from each of the five events of ME07198 were grown until ten days post-bolting. Aerial tissues from Finale™-resistant plants of each event were pooled, frozen in liquid nitrogen, and stored at −80° C. The frozen tissues were lyophilized for 72 hours and stored at −80° C. The freeze-dried tissues were crushed into a fine powder. A 30 mg aliquot of each sample was weighed and placed in a 5 mL microwave extraction vial. Ethyl acetate (1.0 mL) was added to the extraction vial and the mixture was heated to 70° C. for two minutes with stirring. A Biotage Initiator 2.0 microwave extractor (Biotage, Charlottesville, Va.) was used to extract tocopherols, with the microwave power set to 50 watts for the extraction temperature. The extracts were analyzed using GC-MS as described above.

The GC-MS analysis showed that Finale™-resistant T₂ plants from events -02 and -04 had significantly increased α-, β-, δ-, and γ-tocopherol levels compared to control plants. As presented in Table 17, α-tocopherol levels were increased to 130% and 114% in events -02 and -04, respectively, compared to control plants. As presented in Table 18, β-tocopherol levels were increased to 143% and 138% in events -02 and -04, respectively, compared to control plants. As presented in Table 19, δ-tocopherol levels were increased to 143% and 191% in events -02 and -04, respectively, compared to control plants. As presented in Table 20, γ-tocopherol levels were increased to 138% and 136% in events -02 and -04, respectively, compared to corresponding control plants.

TABLE 17
α-Tocopherol levels (% Control) in T₂ plants from ME07198 events

| | Event- | Event- | Event- | | | |
|---|---|---|---|---|---|---|
| | 01 | 02 | 03 | Event-04 | Event-05 | Control |
| T₂ | 113 ± 2 | 130 ± 9 | 68 ± 6 | 114 ± 3 | 102 ± 5 | 100 ± 12 |
| p-value | 0.13 | <0.01 | <0.01 | 0.03 | 0.91 | N/A |

TABLE 18
β-Tocopherol levels (% Control) in T₂ plants from ME07198 events

| | | Event- | Event- | | Event- | |
|---|---|---|---|---|---|---|
| | Event-01 | 02 | 03 | Event-04 | 05 | Control |
| T₂ | 143 ± 18 | 143 ± 9 | 127 ± 7 | 138 ± 16 | 109 ± 6 | 100 ± 19 |
| p-value | 0.03 | <0.01 | 0.01 | 0.03 | 0.31 | N/A |

TABLE 19
δ-Tocopherol levels (% Control) in T₂ plants from ME07198 events

| | | Event- | Event- | Event- | | |
|---|---|---|---|---|---|---|
| | Event-01 | 02 | 03 | 04 | Event-05 | Control |
| T₂ | 136 ± 14 | 143 ± 7 | 53 ± 10 | 191 ± 8 | 66 ± 12 | 100 ± 10 |
| p-value | 0.02 | <0.01 | <0.01 | <0.01 | 0.02 | N/A |

TABLE 20
γ-Tocopherol levels (% Control) in T₂ plants from ME07198 events

| | | Event- | Event- | Event- | | |
|---|---|---|---|---|---|---|
| | Event-01 | 02 | 03 | 04 | Event-05 | Control |
| T₂ | 139 ± 11 | 138 ± 4 | 52 ± 2 | 136 ± 7 | 89 ± 4 | 100 ± 13 |
| p-value | 0.01 | <0.01 | <0.01 | <0.01 | 0.02 | N/A |

The α-tocopherol level in event -03 was significantly decreased compared to control plants. As presented in Table 17, the α-tocopherol level was decreased to 68% in event -03 compared to control plants.

The β-tocopherol levels in events -01 and -03 were significantly increased compared to control plants. As presented in Table 18, the (3-tocopherol levels were increased to 143% and 127% in events -01 and -03, respectively, compared to control plants.

The δ- and γ-tocopherol levels in event -01 were significantly increased compared to control plants. As presented in Table 19, the δ-tocopherol level was increased to 136% in event -01 compared to control plants. As presented in Table 20, the γ-tocopherol level was increased to 139% in event -01 compared to control plants.

The δ- and γ-tocopherol levels in events -03 and -05 were significantly decreased compared to control plants. As presented in Table 19, δ-tocopherol levels were decreased to 53% and 66% in events -03 and -05, respectively, compared to control plants. As presented in Table 20, γ-tocopherol levels were decreased to 52% and 89% in events -03 and -05, respectively, compared to control plants.

### Example 9

### Determination of Functional Homolog and/or Ortholog Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides were designated as a cluster.

The main Reciprocal BLAST process consists of two rounds of BLAST searches; forward search and reverse search. In the forward search step, a query polypeptide sequence, “polypeptide A,” from source species S^{A} was BLASTed against all protein sequences from a species of interest. Top hits were determined using an E-value cutoff of 10⁻⁵ and an identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species S^{A}. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:2 are shown in FIG. 7 and percent identities are shown below in Table 21. Representative functional homologs and/or orthologs for SEQ ID NO:25 are shown in FIG. 8 and percent identities are shown below in Table 22. Representative functional homologs and/or orthologs for SEQ ID NO:32 are shown in FIG. 9 and percent identities are shown below in Table 23. Representative functional homologs and/or orthologs for SEQ ID NO:48 are shown in FIG. 10 and percent identities are shown below in Table 24. Representative functional homologs and/or orthologs for SEQ ID NO:64 are shown in FIG. 11 and percent identities are shown below in Table 25. Representative functional homologs and/or orthologs for SEQ ID NO:77 are shown in FIG. 12 and percent identities are shown below in Table 26. Representative functional homologs and/or orthologs for SEQ ID NO:88 are shown in FIG. 13 and percent identities are shown below in Table 27.

TABLE 21
Percent identity to Ceres clone 19143 (SEQ ID NO: 2)

| | | SEQ | | |
|---|---|---|---|---|
| | | ID | % | |
| Designation | Species | NO: | Identity | E-value |
| Ceres clone 1061027 | Zea mays | 3 | 95.5 | 0 |
| SEQ ID NO: 27 of | Brassica napus | 15 | 94.69 | 8.60E−180 |
| U.S. Patent | | | | |
| Application | | | | |
| No. 20030150015 | | | | |
| Ceres clone 480158 | Glycine max | 4 | 82.1 | 0 |
| Ceres clone 656984 | Glycine max | 5 | 80.8 | 0 |
| gi\|50934645 | Oryza sativa | 6 | 80.3 | 2.1E−129 |
| | (japonica) | | | |
| SEQ ID NO: 25 of | Glycine max | 13 | 79.77 | 7.10E−153 |
| U.S. Patent | | | | |
| Application No. | | | | |
| 20030150015 | | | | |
| Ceres CLONE ID no. | Gossypium | 17 | 79.3 | 3.10E−142 |
| 183492 | hirsutum | | | |
| SEQ ID NO: 23 of | Gossypium | 11 | 79.13 | 7.30E−151 |
| U.S. Patent | hirsutum | | | |
| Application No. | | | | |
| 20030150015 | | | | |
| Ceres CLONE ID no. | Gossypium | 19 | 78.4 | 9.50E−141 |
| 1925254 | hirsutum | | | |
| Ceres CLONE ID no. | Panicum | 21 | 77.5 | 2.70E−127 |
| 1792831 | virgatum | | | |
| Ceres CLONE ID no. | Panicum | 23 | 76.8 | 4.90E−121 |
| 1804277 | virgatum | | | |
| SEQ ID NO: 24 of | Allium porrum | 12 | 76.67 | 2.60E−139 |
| U.S. Patent | | | | |
| Application No. | | | | |
| 20030150015 | | | | |
| gi\|1419090 | Nicotiana | 7 | 76.6 | 0 |
| | tabacum | | | |
| gi\|21228 | Spinacia oleracea | 8 | 76.1 | 0 |
| SEQ ID NO: 26 of | Oryza sativa | 14 | 74.47 | 3.10E−136 |
| U.S. Patent | | | | |
| Application No. | | | | |
| 20030150015 | | | | |
| SEQ ID NO: 22 of | Zea mays | 10 | 73.86 | 1.90E−136 |
| U.S. Patent | | | | |
| Application No. | | | | |
| 20030150015 | | | | |
| gi\|37265798 | Chlamydomonas | 9 | 66.7 | 8.9E−108 |
| | reinhardtii | | | |

TABLE 22
Percent identity to Ceres clone 92102 (SEQ ID NO: 25)

| | | SEQ ID | | |
|---|---|---|---|---|
| Designation | Species | NO: | % Identity | E-value |
| Ceres clone 965028 | Brassica napus | 26 | 58.8 | 1.6E−46 |
| gi\|45642990 | Lycopersicon | 27 | 49.8 | 1.8E−36 |
| | esculentum | | | |
| gi\|40060531 | Vitis aestivalis | 28 | 47.9 | 7.2E−42 |
| gi\|38260618 | Sisymbrium irio | 29 | 46.8 | 6.7E−29 |
| Ceres clone 548557 | Glycine max | 30 | 46.4 | 6.5E−41 |

TABLE 23
Percent identity to Ceres cDNA 23495742 (SEQ ID NO: 32)

| | | SEQ ID | % | |
|---|---|---|---|---|
| Designation | Species | NO: | Identity | E-value |
| gi\|57999638 | Closterium | 35 | 54.9 | 7.2E−19 |
| | peracerosum- | | | |
| | strigosum-littorale | | | |
| | complex | | | |
| Ceres clone | Brassica napus | 36 | 51.5 | 3.3E−17 |
| 1067477 | | | | |
| gi\|42795299 | Mimulus lewisii | 46 | 51 | 3.9E−08 |
| gi\|27372827 | Ipomoea nil | 41 | 50.9 | 1.1E−11 |
| gi\|27372831 | Perilla frutescens | 42 | 50.9 | 3E−11 |
| gi\|27372829 | Perilla frutescens | 43 | 50.9 | 8.1E−11 |
| gi\|45533872 | Brassica oleracea | 38 | 47.9 | 1.6E−12 |
| gi\|45533888 | Brassica oleracea | 39 | 47.9 | 1.6E−12 |
| | var. italica | | | |
| gi\|34922009 | Populus | 44 | 47.4 | 3.5E−10 |
| | yunnanensis | | | |
| gi\|34922000 | Populus | 45 | 47.4 | 4.5E−10 |
| | yunnanensis | | | |
| gi\|45533884 | Brassica oleracea | 40 | 46.5 | 7E−12 |
| | var. gongylodes | | | |
| Ceres clone 681294 | Glycine max | 33 | 45.4 | 3.9E−27 |
| Ceres clone | Parthenium | 37 | 45.1 | 1.3E−12 |
| 1604678 | argentatum | | | |
| Ceres clone 244495 | Zea mays | 34 | 43.7 | 7.9E−20 |

TABLE 24
Percent identity to Ceres ANNOT ID 567302 (SEQ ID NO: 48)

| | | SEQ ID | % | |
|---|---|---|---|---|
| Designation | Species | NO: | Identity | E-value |
| Ceres CLONE ID | Brassica napus | 49 | 89.3 | 4.60E−244 |
| no. 1109488 | | | | |
| Public GI no. | Eucalyptus gunnii | 50 | 76.3 | 2.10E−193 |
| 33188419 | | | | |
| Ceres CLONE ID | Gossypium | 52 | 70.3 | 2.29E−194 |
| no. 1948913 | hirsutum | | | |
| Public GI no. | Helianthus annuus | 53 | 70.1 | 6.39E−183 |
| 80971684 | | | | |
| Ceres CLONE ID | Glycine max | 54 | 70.1 | 2.30E−187 |
| no. 1245537 | | | | |
| Public GI no. | Helianthus annuus | 55 | 69.9 | 2.20E−182 |
| 80971690 | | | | |
| Ceres ANNOT ID | Populus | 57 | 68.9 | 2.40E−183 |
| no. 1530974 | balsamifera subsp. | | | |
| | trichocarpa | | | |
| Ceres CLONE ID | Glycine max | 58 | 68.9 | 1.19E−188 |
| no. 574132 | | | | |
| Public GI no. | Solanum tuberosum | 59 | 66.5 | 1.80E−178 |
| 47078321 | | | | |
| Public GI no. | Oryza sativa subsp. | 60 | 65.3 | 6.89E−170 |
| 50906901 | japonica | | | |
| Ceres CLONE ID | Triticum aestivum | 61 | 64.6 | 3.49E−175 |
| no. 754013 | | | | |
| Public GI no. | Triticum aestivum | 62 | 64.2 | 6.50E−174 |
| 91694297 | | | | |

TABLE 25
Percent identity to Ceres ANNOT ID 552252 (SEQ ID NO: 64)

| | | SEQ | | |
|---|---|---|---|---|
| | | ID | % | |
| Designation | Species | NO: | Identity | E-value |
| Public GI no. 81295666 | Glycine max | 65 | 77.7 | 1.20E−133 |
| Public GI no. 51949754 | Medicago | 66 | 76.2 | 9.29E−127 |
| | sativa | | | |
| Public GI no. 92882118 | Medicago | 67 | 75.5 | 3.99E−126 |
| | truncatula | | | |
| Public GI no. 61808320 | Glycine max | 68 | 75.5 | 8.99E−129 |
| Public GI no. 51536170 | Oryza sativa | 69 | 74.7 | 8.39E−110 |
| | subsp. japonica | | | |
| Ceres CLONE ID no. | Panicum | 71 | 74.5 | 1.10E−121 |
| 1789748 | virgatum | | | |
| Ceres CLONE ID no. | Zea mays | 72 | 73 | 1.59E−122 |
| 395119 | | | | |
| Public GI no. 81295658 | Zea mays | 73 | 73 | 3.30E−122 |
| Ceres ANNOT ID no. | Populus | 75 | 70.4 | 2.59E−115 |
| 1478147 | balsamifera | | | |
| | subsp. | | | |
| | trichocarpa | | | |

TABLE 26
Percent identity to Ceres ANNOT ID no. 859061 (SEQ ID NO: 77)

| | | SEQ | | |
|---|---|---|---|---|
| | | ID | % | |
| Designation | Species | NO: | Identity | E-value |
| Public GI no. | Artificial Sequence | 79 | 80.4 | 2.50E−38 |
| 51949754_T | | | | |
| Public GI no. | Artificial Sequence | 80 | 79.3 | 8.40E−38 |
| 92882118_T | | | | |
| Ceres CLONE ID no. | Artificial Sequence | 83 | 74.7 | 5.30E−36 |
| 1789748_T | | | | |
| Public GI no. | Artificial Sequence | 78 | 74.3 | 1.50E−40 |
| 81295666_T | | | | |
| Public GI no. | Artificial Sequence | 81 | 70.6 | 5.20E−38 |
| 61808320_T | | | | |
| Ceres CLONE ID no. | Artificial Sequence | 84 | 67.9 | 3.30E−36 |
| 395119_T | | | | |
| Public GI no. | Artificial Sequence | 85 | 67.9 | 3.30E−36 |
| 81295658_T | | | | |
| Public GI no. | Artificial Sequence | 82 | 58.2 | 1.50E−24 |
| 51536170_T | | | | |
| Ceres ANNOT ID no. | Artificial Sequence | 86 | 53.5 | 2.30E−44 |
| 1478147_T | | | | |

TABLE 27
Percent identity to Ceres CLONE ID no. 125255 (SEQ ID NO: 88)

| | | SEQ ID | % | |
|---|---|---|---|---|
| Designation | Species | NO: | Identity | E-value |
| Public GI no. 7406453 | Arabidopsis | 89 | 98.6 | 3.69E−155 |
| | thaliana | | | |
| Public GI no. 28393229 | Arabidopsis | 90 | 98.3 | 1.29E−154 |
| | thaliana | | | |
| Ceres CLONE ID no. | Zea mays | 91 | 83.1 | 1.30E−127 |
| 1377623 | | | | |
| Ceres ANNOT ID no. | Populus | 93 | 67.7 | 4.69E−84 |
| 1518536 | balsamifera | | | |
| | subsp. | | | |
| | trichocarpa | | | |
| Public GI no. 76443937 | Glycine max | 94 | 63.4 | 9.80E−84 |
| Ceres CLONE ID no. | Glycine max | 95 | 63.4 | 9.80E−84 |
| 464672 | | | | |
| Ceres CLONE ID no. | Gossypium | 97 | 59.3 | 8.49E−85 |
| 1940214 | hirsutum | | | |
| Public GI no. 76443931 | Zea mays | 98 | 58.4 | 4.20E−76 |
| Ceres CLONE ID no. | Zea mays | 99 | 58.4 | 4.20E−76 |
| 287069 | | | | |
| Ceres CLONE ID no. | Panicum | 101 | 58.1 | 2.80E−77 |
| 1780314 | virgatum | | | |
| Public GI no. 76443929 | Zea mays | 102 | 58 | 2.30E−75 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing a plant tissue, said method comprising growing a plant cell comprising an exogenous nucleic acid comprising a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, wherein said tissue has a difference in the level of one or both of a tocopherol and a tocotrienol as compared to the corresponding level in tissue of a control plant that does not comprise said nucleic acid.

2. A method of producing a plant tissue, said method comprising growing a plant cell comprising at least two nucleotide sequences, wherein each nucleotide sequence encodes a polypeptide having 80% or greater sequence identity to an amino acid sequence selected from the group consisting of:
(a) SEQ ID NOs:2-15, SEQ ID NO:17, SEQ ID NO:19, SEQ ID NO:21, SEQ ID NO:23, and the consensus sequence set forth in FIG. 7;
(b) SEQ ID NOs:25-30 and the consensus sequence set forth in FIG. 8;
(c) SEQ ID NOs:32-46 and the consensus sequence set forth in FIG. 9;
(d) SEQ ID NOs:48-50, SEQ ID NOs:52-55, SEQ ID NOs:57-62, and the consensus sequence set forth in FIG. 10;
(e) SEQ ID NOs:64-69, SEQ ID NOs:71-73, SEQ ID NO:75, and the consensus sequence set forth in FIG. 11;
(f) SEQ ID NOs:77-86 and the consensus sequence set forth in FIG. 12; and
(g) SEQ ID NOs:88-91, SEQ ID NOs:93-95, SEQ ID NOs:97-99, SEQ ID NOs:101-102, and the consensus sequence set forth in FIG. 13;
wherein each of said at least two nucleotide sequences is from a different one of (a), (b), (c), (d), (e), (f), or (g); and wherein said tissue has a difference in the level of one or both of a tocopherol and a tocotrienol as compared to the corresponding level in tissue of a control plant that does not comprise said at least two nucleotide sequences.

3. The method claim 1 or 2, wherein each said sequence identity is 85% or greater.

4. The method of claim 3, wherein each said sequence identity is 90% or greater.

5. The method of claim 4, wherein each said sequence identity is 95% or greater.

6. The method of claim 1, wherein said nucleotide sequence encodes a polypeptide comprising an amino acid sequence corresponding to SEQ ID NO:3, SEQ ID NO:25, SEQ ID NO:32, SEQ ID NO:48, SEQ ID NO:64, SEQ ID NO:77, or SEQ ID NO:88.

7-12. (canceled)

13. The method of claim 1, wherein said nucleotide sequence encodes a polypeptide comprising an amino acid sequence corresponding to the consensus sequence set forth in any of FIGS. 7-13.

14. The method of claim 1 or 2, wherein said difference is an increase in the level of a tocopherol.

15. The method of claim 1 or 2, wherein said difference is an increase in the level of a tocotrienol.

16. The method of claim 1, wherein said exogenous nucleic acid is operably linked to a regulatory region.

17. The method of claim 16, wherein said regulatory region is a cell-specific or tissue-specific promoter.

18. The method of claim 17, wherein said promoter is a seed-specific promoter.

19. The method of claim 18, wherein said seed-specific promoter is selected from the group consisting of the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, and the barley hordein gene promoter.

20. The method of claim 16, wherein said regulatory region is a broadly expressing promoter.

21. The method of claim 20, wherein said broadly expressing promoter is selected from the group consisting of p326 (SEQ ID NO:178), YP0158 (SEQ ID NO:159), YP0214 (SEQ ID NO:163), YP0380 (SEQ ID NO:172), PT0848 (SEQ ID NO:128), PT0633 (SEQ ID NO:109), YP0050 (SEQ ID NO:137), YP0144 (SEQ ID NO:157), and YP0190 (SEQ ID NO:161).

22. The method of claim 16, wherein said regulatory region is a constitutive promoter.

23. The method of claim 16, wherein said regulatory region is an inducible promoter.

24. The method of claim 2, wherein each of said at least two nucleotide sequences is operably linked to a regulatory region.

25. The method of claim 24, wherein said regulatory regions are cell-specific or tissue-specific promoters.

26. The method of claim 24, wherein said regulatory regions are seed-specific promoters.

27. The method of claim 24, wherein said regulatory regions are broadly expressing promoters.

28. The method of claim 24, wherein said regulatory regions are constitutive promoters.

29. The method of claim 24, wherein said regulatory regions are inducible promoters.

30. The method of claim 1 or 2, wherein said plant is from a genus selected from the group consisting of Acokanthera, Aesculus, Anamirta, Ananas, Arachis, Betula, Bixa, Brassica, Calendula, Carthamus, Centella, Chrysanthemum, Cinnamomum, Citrullus, Coffea, Convallaria, Curcuma, Cymbopogon, Daphne, Elaeis, Euphorbia, Fragaria, Glycine, Glycyrrhiza, Gossypium, Helianthus, Isodon, Lactuca, Lavandula, Linum, Luffa, Lycopersicon, Mentha, Musa, Ocimum, Origanum, Oryza, Rabdosia, Ricinus, Rosmarinus, Ruscus, Salvia, Sesamum, Solanum, Strophanthus, Theobroma, Thymus, Triticum, Vitis, and Zea.

31. The method of claim 1 or 2, wherein said plant is a species selected from Ananas comosus, Bixa orellana, Brassica campestris, Brassica napus, Brassica oleracea, Calendula officinalis, Chrysanthemum parthenium, Cinnamomum camphora, Coffea arabica, Glycine max, Glycyrrhiza glabra, Gossypium spp., Lactuca sativa, Lycopersicon esculentum, Mentha piperita, Mentha spicata, Musa paradisiaca, Oryza sativa, Rosmarinus officinalis, Solanum tuberosum, Theobroma cacao, Triticum aestivum, Vitis vinifera, and Zea mays.

32. The method of claim 1 or 2, wherein said plant is selected from the group consisting of peanut, safflower, flax, sugar beet, chick peas, alfalfa, spinach, clover, cabbage, lentils, mustard, soybean, lettuce, castor bean, sesame, carrot, grape, cotton, crambe, strawberry, amaranth, high erucic acid canola, broccoli, peas, pepper, tomato, potato, kidney beans, lima beans, dry beans, green beans, watermelon, cantaloupe, peach, pear, apple, cherry, orange, lemon, grapefruit, plum, mango, oilseed rape, sunflower, garlic, oil palm, date palm, banana, sweet corn, popcorn, field corn, wheat, rye, barley, oat, onion, pineapple, rice, millet, and sorghum.

33. The method of any of claim 1 or 2, wherein said tissue is leaf tissue.

34. The method of any of claim 1 or 2, wherein said tissue is seed tissue.

35. The method of any of claim 1 or 2, wherein said tissue is fruit tissue.

36. The method of any of claim 1 or 2, wherein said tissue is a tissue culture.

37. An isolated nucleic acid molecule comprising a nucleotide sequence having 95% or greater sequence identity to the nucleotide sequence set forth in SEQ ID NO:16, SEQ ID NO:18, SEQ ID NO:20, SEQ ID NO:22, SEQ ID NO:51, SEQ ID NO:56, SEQ ID NO:70, SEQ ID NO:74, SEQ ID NO:92, SEQ ID NO:96, or SEQ ID NO:100.

38. An isolated nucleic acid comprising a nucleotide sequence encoding a polypeptide having 80% or greater sequence identity to the amino acid sequence set forth in SEQ ID NO:17, SEQ ID NO:19, SEQ ID NO:21, SEQ ID NO:23, SEQ ID NO:52, SEQ ID NO:57, SEQ ID NO:71, SEQ ID NO:75, SEQ ID NO:93, SEQ ID NO:97, or SEQ ID NO:101.

39-58. (canceled)

59. A plant comprising an exogenous nucleic acid comprising a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, wherein one or more tissues of said plant have a difference in the level of one or both of a tocopherol and a tocotrienol as compared to the corresponding level in tissue of a control plant that does not comprise said nucleic acid.

60. A plant comprising at least two nucleotide sequences, wherein each nucleotide sequence encodes a polypeptide having 80% or greater sequence identity to an amino acid sequence selected from the group consisting of:
(a) SEQ ID NOs:2-15, SEQ ID NO:17, SEQ ID NO:19, SEQ ID NO:21, SEQ ID NO:23, and the consensus sequence set forth in FIG. 7;
(b) SEQ ID NOs:25-30 and the consensus sequence set forth in FIG. 8;
(c) SEQ ID NOs:32-46 and the consensus sequence set forth in FIG. 9;
(d) SEQ ID NOs:48-50, SEQ ID NOs:52-55, SEQ ID NOs:57-62, and the consensus sequence set forth in FIG. 10;
(e) SEQ ID NOs:64-69, SEQ ID NOs:71-73, SEQ ID NO:75, and the consensus sequence set forth in FIG. 11;
(f) SEQ ID NOs:77-86 and the consensus sequence set forth in FIG. 12; and
(g) SEQ ID NOs:88-91, SEQ ID NOs:93-95, SEQ ID NOs:97-99, SEQ ID NOs:101-102, and the consensus sequence set forth in FIG. 13;
wherein each of said at least two nucleotide sequences is from a different one of (a), (b), (c), (d), (e), (f), or (g); and wherein one or more tissues of said plant have a difference in the level of one or both of a tocopherol and a tocotrienol as compared to the corresponding level in tissue of a control plant that does not comprise said at least two nucleotide sequences.

61. The plant of claim 59 or 60, wherein said difference is an increase in said level of one or both of a tocopherol and a tocotrienol.

62. The plant of claim 59 or 60, wherein said difference is an increase in the level of a tocopherol.

63. Seed from a plant according to claim 61.

64. Non-seed tissue from a plant according to claim 61.

65. Oil from the seed of claim 63.

66. The oil of claim 65, wherein said oil demonstrates an increased oxidative stability in the absence of added antioxidants relative to oil derived from seed of a control plant in the absence of added antioxidants.

67. A food product comprising seed according to claim 63.

68. A food product comprising non-seed tissue according to claim 64.

69. A method of producing oil having an increased oxidative stability in the absence of added antioxidants, said method comprising extracting oil from seed according to claim 63.

70. A method of enhancing the nutritional value of a food product, said method comprising including tissue from a plant according to claim 59 or 60 in said food product.

71. A method of making a plant, comprising:
a) obtaining a plurality of plants transformed with an exogenous nucleic acid, said exogenous nucleic acid comprising a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, said nucleotide sequence operably linked to a regulatory region; and
b) selecting from said plurality of plants at least one plant in which one or more tissues of said plant have a difference in the level of one or both of a tocopherol and a tocotrienol as compared to the corresponding level in tissue of a control plant that does not comprise said nucleic acid.

72. A method of enhancing the nutritional value of a plant, said method comprising growing a plant comprising an exogenous nucleic acid comprising a nucleotide sequence encoding a polypeptide having 80% or greater sequence identity to an amino acid sequence selected from the group consisting of SEQ ID NOs:2-15, SEQ ID NO:17, SEQ ID NO:19, SEQ ID NO:21, SEQ ID NO:23, SEQ ID NOs:25-30, SEQ ID NOs:32-46, SEQ ID NOs:48-50, SEQ ID NOs:52-55, SEQ ID NOs:57-62, SEQ ID NOs:64-69, SEQ ID NOs:71-73, SEQ ID NO:75, SEQ ID NOs:77-86, SEQ ID NOs:88-91, SEQ ID NOs:93-95, SEQ ID NOs:97-99, SEQ ID NOs:101-102, and the consensus sequences set forth in FIGS. 7-13, wherein a tissue of said plant has an increased level of one or both of a tocopherol and a tocotrienol as compared to the corresponding level in tissue of a control plant that does not comprise said nucleic acid.

73. A method of enhancing the nutritional value of a plant, said method comprising growing a plant comprising at least two nucleotide sequences, wherein each nucleotide sequence encodes a polypeptide having 80% or greater sequence identity to an amino acid sequence selected from the group consisting of:
(a) SEQ ID NOs:2-15, SEQ ID NO:17, SEQ ID NO:19, SEQ ID NO:21, SEQ ID NO:23, and the consensus sequence set forth in FIG. 7;
(b) SEQ ID NOs:25-30 and the consensus sequence set forth in FIG. 8;
(c) SEQ ID NOs:32-46 and the consensus sequence set forth in FIG. 9;
(d) SEQ ID NOs:48-50, SEQ ID NOs:52-55, SEQ ID NOs:57-62, and the consensus sequence set forth in FIG. 10;
(e) SEQ ID NOs:64-69, SEQ ID NOs:71-73, SEQ ID NO:75, and the consensus sequence set forth in FIG. 11;
(f) SEQ ID NOs:77-86 and the consensus sequence set forth in FIG. 12; and
(g) SEQ ID NOs:88-91, SEQ ID NOs:93-95, SEQ ID NOs:97-99, SEQ ID NOs:101-102, and the consensus sequence set forth in FIG. 13;
wherein each of said at least two nucleotide sequences is from a different one of (a), (b), (c), (d), (e), (f), or (g); and wherein a tissue of said plant has an increased level of one or both of a tocopherol and a tocotrienol as compared to the corresponding level in tissue of a control plant that does not comprise said at least two nucleotide sequences.
